(19) United States
(12) Patent Application Publication
(10) Pub. No.: US 2026/0034118 A1
(43) Pub. Date: Feb. 5, 2026

(54) TREATMENT OF COPD PATIENT POPULATIONS

(71) Applicant: Kinaset Therapeutics, Inc., Medfield, MA (US)

(72) Inventors: Frazer Giles Morgan, Chippenham (GB); Christopher D. O’Brien, Medfield, MA (US)

(21) Appl. No.: 19/286,896

(22) Filed: Jul. 31, 2025

Related U.S. Application Data
(60) Provisional application No. 63/677,685, filed on Jul. 31, 2024.

(51) Int. Cl. A61K 31/4545; A61P 11/06; C07D 519/00

(52) U.S. Cl. CPC A61K 31/4545; A61P 11/06; C07D 519/00

(57) ABSTRACT

The present disclosure relates to methods of treatment of asthma, COPD, Asthma-COPD Overlap Syndrome (ACOS), and chronic bronchitis, or a combination thereof, with formulations comprising (S)-3-(3-(1-methyl-2-oxo-5-(pyrazolo[1,5-a]pyridin-3-yl)-1,2-dihydro-3H-imidazo[4,5-b]pyridin-3-yl)piperidin-1-yl)-3-oxopropanenitrile.

### CROSS-REFERENCE TO RELATED APPLICATIONS

This application claims priority to U.S. Provisional Application Ser. No. 63/677,685 filed Jul. 31, 2024, which is hereby incorporated herein by reference in its entirety.

### BACKGROUND

International application WO 2011/051452 discloses compounds that are useful as Janus kinase inhibitors; the application describes JAK1, JAK2, JAK3, and TYK2 inhibitors. The compounds disclosed therein have utility in the treatment of various diseases, including respiratory indications such as asthma and COPD, as well as other inflammatory processes that may be associated with eosinophilic or non-eosinophilic inflammation.

Drugs for the treatment of respiratory diseases are frequently administered via dry powder inhalation devices. Formulating respiratory drugs as dry powders with inhalation excipients such as lactose is complicated and unpredictable. There is a continuing need for stable dry powder formulations that exhibit desirable bioavailability and physical properties as well as corresponding methods of treatment with stable dry powder formulations. Physical characteristics are important for efficient handling and processing of the drug substance and formulation (or pharmaceutical formulation or blend or inhalation powder if that is the term used herein), to ensure that an effective dose is delivered to the correct part of the lung, and that the drug is effective in treating respiratory diseases. Different formulation techniques are known in the art and can be applied to drug compounds to produce inhalation powders having the desired drug delivery properties.

There is also an ongoing need to treat asthma and COPD, as well as inflammatory processes associated with eosinophilic inflammation or non-eosinophilic inflammation.

### SUMMARY

Disclosed herein are methods of treating a disease or disorder in a subject in need thereof, wherein the method comprises administering to the subject a JAK inhibitor or a pharmaceutical formulation comprising a JAK inhibitor, and wherein the disease or disorder is selected from asthma, COPD, Asthma-COPD Overlap Syndrome (ACOS), and chronic bronchitis, or a combination thereof.

In particular, disclosed herein are methods of treating asthma, COPD, Asthma-COPD Overlap Syndrome (ACOS), and chronic bronchitis, or a combination thereof, with (S)-3-(3-(1-methyl-2-oxo-5-(pyrazolo[1,5-a]pyridine-3-yl)-1H-imidazo[4,5-b]pyridine-3(2H)-yl)piperidin-1-yl)-3-oxopropanenitrile (Compound I) and formulations comprising Compound I,

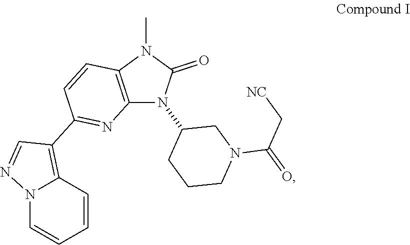

or a pharmaceutically acceptable salt thereof.

The methods of treatment provided herein are, at least, useful in treating particular COPD patient populations as demonstrated in clinical trial studies. Thus, in an embodiment, the subject is classified as a part of particular COPD population. In addition, the disclosed methods are unexpected in that Compound I formulations produce greater lung function improvements (e.g., FEV₁ and yFEF₂₅₋₇₅) in subjects with T2 low COPD (blood eosinophils <300 cells/μL) than in subjects with T2 high COPD (blood eosinophils ≥300 cells/μL).

In another embodiment, the method comprises administering Compound I, or formulations comprising Compound I, or a pharmaceutically acceptable salt thereof.

In an aspect, provided herein is a method of treating Asthma-COPD Overlap Syndrome (ACOS) in a subject in need thereof, wherein the method comprises administering to the subject a pharmaceutical formulation comprising Compound I, or a pharmaceutically acceptable salt thereof.

In yet another aspect, provided herein is a method of treating a disease or disorder in a subject in need thereof, wherein the method comprises administering to the subject a pharmaceutical formulation comprising Compound I, or a pharmaceutically acceptable salt thereof, and a pharmaceutically acceptable excipient, wherein:
the disease or disorder is selected from asthma, COPD, Asthma-COPD Overlap Syndrome (ACOS), and chronic bronchitis, or a combination thereof; and
prior to administering the pharmaceutical formulation, the subject has a blood eosinophil level that is less than about 300 cells/μL.

In another aspect, provided herein is a method of treating a disease or disorder in a subject in need thereof, wherein the method comprises administering to the subject a pharmaceutical formulation comprising Compound I, or a pharmaceutically acceptable salt thereof, wherein:
the disease or disorder is selected from COPD and chronic bronchitis, or a combination thereof; and
prior to administering the pharmaceutical formulation, the subject has a blood eosinophil level that is less than about 300 cells/μL.

In an aspect, provided herein is a method of treating COPD in a subject in need thereof, wherein the method comprises administering to the subject a pharmaceutical formulation comprising Compound I, or a pharmaceutically acceptable salt thereof, wherein:
prior to administering the pharmaceutical formulation, the subject has a blood eosinophil level that is less than about 300 cells/μL.

In yet another aspect, provided herein is a method of treating a disease or disorder in a subject in need thereof, wherein the method comprises administering to the subject a pharmaceutical formulation comprising Compound I, or a pharmaceutically acceptable salt thereof, wherein:
the disease or disorder is selected from asthma, COPD, Asthma-COPD Overlap Syndrome (ACOS), and chronic bronchitis, or a combination thereof; and prior to administering the pharmaceutical formulation, the subject has a blood eosinophil level that is less than or equal to 89 cells/μL.

In an embodiment, the subject is diagnosed with dyspnea. In another embodiment, prior to administering the pharmaceutical formulation, the subject's dyspnea according to the modified Medical Research Council (mMRC) scale is 0-4.

In another embodiment, prior to administering the pharmaceutical formulation, the subject has a COPD assessment test (CAT) score of 4 to 30. In yet another embodiment, prior to administering the pharmaceutical formulation, the subject has a CAT score of less than 10. In still another embodiment, prior to administering the pharmaceutical formulation, the subject has an average CAT score of about 15.

In an embodiment, prior to administering the pharmaceutical formulation, the subject's forced expiratory volume in one second (FEV₁) is 40% to 80% of predicted normal. In another embodiment, prior to administering the pharmaceutical formulation, the subject's average FEV₁ is about 60% of predicted normal. In still another embodiment, prior to administering the pharmaceutical formulation, the subject's FEV₁ is 0.8 L to 2.5 L. In an embodiment, prior to administering the pharmaceutical formulation, the subject's average FEV₁ is about 1.7 L.

In an embodiment, prior to administering the pharmaceutical formulation, the subject's pre-bronchodilator forced expiratory volume in one second (FEV₁) is 40% to 80% of predicted normal. In another embodiment, prior to administering the pharmaceutical formulation, the subject's average pre-bronchodilator FEV₁ is about 60% of predicted normal. In still another embodiment, prior to administering the pharmaceutical formulation, the subject's pre-bronchodilator FEV₁ is 0.8 L to 2.5 L. In an embodiment, prior to administering the pharmaceutical formulation, the subject's average pre-bronchodilator FEV₁ is about 1.7 L.

In another embodiment, prior to administering the pharmaceutical formulation, the subject's ratio of FEV₁ to forced vital capacity (FEV₁/FVC) is about 0.35 to about 0.70. In another embodiment, prior to administering the pharmaceutical formulation, the subject's FEV₁/FVC is about 0.40 to about 0.70. In yet another embodiment, prior to administering the pharmaceutical formulation, the subject's average FEV₁/FVC is about 0.55.

In another embodiment, prior to administering the pharmaceutical formulation, the subject's ratio of post-bronchodilator FEV₁ to forced vital capacity (FEV₁/FVC) is 0.40 to 0.70. In yet another embodiment, prior to administering the pharmaceutical formulation, the subject's average post-bronchodilator FEV₁/FVC is about 0.55.

In still another embodiment, prior to administering the pharmaceutical formulation, the subject's blood eosinophil count as expressed in units of cells/μL is 70-480. In an embodiment, prior to administering the pharmaceutical formulation, the subject's average blood eosinophil count as expressed in units of cells/μL is about 235.

In another embodiment, prior to administering the pharmaceutical formulation, the subject's fractional exhaled nitric oxide (FeNO) concentration is 5.0 ppb to 75.0 ppb. In an embodiment, prior to administering the pharmaceutical formulation, the subject's average FeNO concentration is about 30 ppb.

In another embodiment, prior to administering the pharmaceutical formulation, the subject's breathlessness, cough, and sputum scale (BCSS) score is 1-8. In yet another embodiment, prior to administering the pharmaceutical formulation, the subject's average BCSS score is 3.

In still another embodiment, prior to administering the pharmaceutical formulation, the subject is undergoing treatment with a background therapy selected from an inhaled corticosteroid, an inhaled long-acting beta-agonist, and an inhaled long-acting muscarinic antagonist, or a combination of two or more thereof.

In an embodiment, after administering the pharmaceutical formulation, the subject's placebo-corrected percent change in FeNO concentration is −10.0% to −90.0%. In another embodiment, after administering the pharmaceutical formulation, the subject's average placebo-corrected percent change in FeNO concentration is about −30.0% to about −50.0%.

In yet another embodiment, after administering the pharmaceutical formulation, the subject's placebo-corrected change in FeNO concentration is −1.5 ppb to −45.0 ppb. In still another embodiment, after administering the pharmaceutical formulation, the subject's average placebo-corrected change in FeNO concentration is about −1.0 ppb to about −20.0 ppb.

In an embodiment, after administering the pharmaceutical formulation, the subject's change in FEV₁ is +350 to +700 mL. In another embodiment, after administering the pharmaceutical formulation, the subject's average placebo-corrected change in FEV₁ is about +120 mL to about +260 mL.

In yet another embodiment, administration of the pharmaceutical formulation produces an exposure of JAK inhibitor in the subject as indicated by AUC_{0-∞} that is 340,000 h·pg/mL to 755,000 h·pg/mL. In still another embodiment, administration of the pharmaceutical formulation produces an average exposure of JAK inhibitor in the subject as indicated by AUC_{0-∞} that is about 555,000 h·pg/mL.

In an embodiment, administration of the pharmaceutical formulation produces an exposure of JAK inhibitor in the subject as indicated by AUC_{0-last non-zero} that is 340,000 h·pg/mL to 755,000 h·pg/mL. In another embodiment, administration of the pharmaceutical formulation produces an average exposure of JAK inhibitor in the subject as indicated by AUC_{0-last non-zero} that is about 555,000 h·pg/mL.

In an embodiment, administration of the pharmaceutical formulation produces an exposure of JAK inhibitor in the subject as indicated by AUC_{over dosing interval} that is 130,000 h·pg/mL to 375,000 h·pg/mL. In another embodiment, administration of the pharmaceutical formulation produces an average exposure of JAK inhibitor in the subject as indicated by AUC_{over dosing interval} that is about 272,000 h·pg/mL.

In yet another embodiment, administration of the pharmaceutical formulation produces an exposure of JAK inhibitor in the subject as indicated by AUC_{0-12 hours} that is 130,000 h·pg/mL to 375,000 h·pg/mL.

In still another embodiment, administration of the pharmaceutical formulation produces an average exposure of JAK inhibitor in the subject as indicated by AUC_{0-12 hours} that is about 272,000 h·pg/mL.

In an embodiment, administration of the pharmaceutical formulation produces a concentration of JAK inhibitor in the subject as indicated by Cₘₐₓ that is 10,000 pg/mL to 45,000 pg/mL. In another embodiment, administration of the pharmaceutical formulation produces an average concentration of JAK inhibitor in the subject as indicated by Cₘₐₓ that is about 33,000 pg/mL.

In yet another embodiment, administration of the pharmaceutical formulation produces a concentration of JAK inhibitor in the subject as indicated by C_{trough} that is 9,000 pg/mL to 20,000 pg/mL. In still another embodiment, administration of the pharmaceutical formulation produces an average concentration of JAK inhibitor in the subject as indicated by C_{trough} that is about 14,000 pg/mL.

In an embodiment, administration of the pharmaceutical formulation produces a half-life (T_{1/2}) of JAK inhibitor in the subject that is 15 hours to 35 hours. In another embodiment, administration of the pharmaceutical formulation produces an average T_{1/2} of JAK inhibitor in the subject that is about 23 hours.

### BRIEF DESCRIPTION OF THE DRAWINGS

FIG. 1 is the sampling plan used for the determination of drug content and content uniformity in Compound I formulations for clinical studies.

### DETAILED DESCRIPTION

Disclosed herein are methods of treating a disease or disorder in a subject in need thereof, wherein the method comprises administering to the subject a pharmaceutical formulation comprising a JAK inhibitor, and wherein the disease or disorder is selected from asthma, COPD, Asthma-COPD Overlap Syndrome (ACOS), and chronic bronchitis, or a combination thereof.

The methods of treatment provided herein are, at least, useful in particular COPD patient populations. Thus, in an embodiment, the subject is classified as a part of particular COPD population.

In another embodiment, the method comprises administering formulations comprising Compound I:

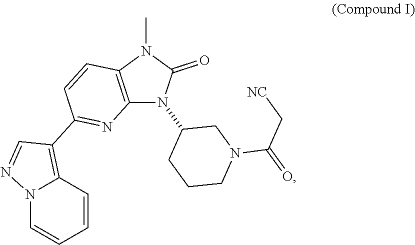

or a pharmaceutically acceptable salt thereof, and a pharmaceutically acceptable excipient.

These methods produce greater lung function improvements (e.g., FEV₁ and FEF₂₅₋₇₅) in subjects with T2 low COPD (blood eosinophils <300 cells/μL) than in subjects with T2 high COPD (blood eosinophils ≥300 cells/μL) and are therefore surprising.

### Definitions

It is to be understood that the terminology used herein is for the purpose of describing particular embodiments only and is not intended to be limiting.

Unless defined otherwise, all technical and scientific terms used herein have the same meaning as commonly understood by one of ordinary skill in the art to which the disclosure pertains. Although other methods and materials similar, or equivalent, to those described herein can be used in the practice of the present disclosure, the preferred materials and methods are described herein.

As used in this specification and the appended claims, the singular forms “a,” “an” and “the” include plural referents unless the context clearly dictates otherwise. Thus, for example, reference to “an excipient” includes a combination of two or more such excipients, reference to “a glidant” includes one or more glidants, or mixtures of glidants, reference to “a filler” includes one or more fillers, or mixtures of fillers, and the like. Unless specifically stated or obvious from context, as used herein, the term “or” is understood to be inclusive and covers both “or” and “and.”

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Thus, for example, an FEV₁ of about 40% of predicted normal includes an FEV₁ that is within the range of 36% to 44%, inclusive of the endpoints, of predicted normal.

As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present technology that do not contain those elements or features. Although the open-ended term “comprising,” as a synonym of terms such as including, containing, or having, is used herein to describe and claim the disclosure, the present technology, or embodiments thereof, may alternatively be described using more limiting terms such as “consisting of” or “consisting essentially of” the recited ingredients.

Unless specifically stated or obvious from context, as used herein, the term “substantially” is understood as within a narrow range of variation or otherwise normal tolerance in the art. Substantially can be understood as within 5%, 4%, 3%, 2%, 1%, 0.5%, 0.1%, 0.05%, 0.01% or 0.001% of the stated value.

“Subject” or “patient” refers to any animal, such as a mammal. Examples of mammals include, but are not limited to, cows, horses, sheep, pigs, cats, dogs, mice, rats, rabbits, guinea pigs, non-human primates (NHPs) such as monkeys or apes, humans, etc., including a human.

“Therapeutically-effective amount” or “effective amount” refers to an amount that is effective to elicit the desired biological or medical response, including the amount of a compound that, when administered to the subject for treating a disease, is sufficient to affect such treatment for the disease. The effective amount will vary depending on the compound, the disease, and its severity and the age, weight, etc., of the subject to be treated. The effective amount can include a range of amounts. As is understood in the art, an effective amount may be in one or more doses, i.e., a single dose or multiple doses may be required to achieve the desired treatment endpoint. An effective amount may be considered in the context of administering one or more therapeutic agents, and a single agent may be considered to be given in an effective amount if, in conjunction with one or more other agents, a desirable or beneficial result may be or is achieved. Suitable doses of any co-administered compounds may optionally be lowered due to the combined action (such as additive or synergistic effects) of the compounds.

As used herein, the term “treating” or “treatment” refers to inhibiting a disease; for example, inhibiting a disease, condition, or disorder in an individual who is experiencing or displaying the pathology or symptomology of the disease, condition, or disorder (i.e., arresting further development of the pathology and/or symptomology) or ameliorating the disease; for example, ameliorating a disease, condition, or disorder in an individual who is experiencing or displaying the pathology or symptomology of the disease, condition, or disorder (i.e., reversing the pathology and/or symptomology) such as decreasing the severity of the disease.

The term “prevent,” “preventing,” or “prevention” as used herein, comprises the prevention of at least one symptom associated with or caused by the state, disease or disorder being prevented.

As used herein, the term “background therapy” refers to a standard-of-care therapy used in the treatment of, for example, asthma and COPD. It is to be understood that when a subject is undergoing treatment with a background therapy, the subject is undergoing simultaneous treatment with a standard-of-care therapy in addition to treatment with Compound I, or pharmaceutical formulations comprising Compound I. In some embodiments, the background therapy is selected from the group consisting of an inhaled corticosteroid (ICS), an inhaled long-acting beta-agonist (LABA), and an inhaled long-acting muscarinic antagonist (LAMA), or a combination of two or more thereof.

According to the methods of treatment of the present disclosure, disorders are treated or prevented in a subject, such as a human or other animal, by administering to the subject a therapeutically effective amount of a compound of the disclosure, in such amounts and for such time as is necessary to achieve the desired result. The term “therapeutically effective amount” of a compound of the disclosure, as used herein, means enough of the compound to decrease the symptoms of a disorder in a subject. As is well understood in the medical arts a therapeutically effective amount of a compound of this disclosure will be at a reasonable benefit/risk ratio applicable to any medical treatment.

As used herein, the term “asthma-COPD overlap syndrome (ACOS)” is used to describe patients that exhibit symptoms of both asthma and COPD. Patients with ACOS may exhibit persistent airflow obstruction with features of asthma (e.g., wheezing, chest tightness, and dry cough, each of which may worsen at night).

As used herein, the terms “inhalation powder,” “formulation,” “pharmaceutical formulation,” and “dry powder pharmaceutical formulation” refer to a blend, aggregation, solution or other combination of materials which includes an active pharmaceutical ingredient (API). In a non-limiting example, the formulation includes an API and one or more pharmaceutically acceptable excipients, i.e., as used herein, “inhalation powder,” “formulation,” “pharmaceutical formulation,” and “dry powder pharmaceutical formulation” comprises Compound I, or a pharmaceutically acceptable salt thereof, and one or more pharmaceutically acceptable excipients. Examples of pharmaceutically acceptable excipients include, but are not limited to, lactose and magnesium stearate.

As used herein, the term “combining” or “combined” is used to refer to the action of adding two or more components together to form a mixture or preblend. This definition implies that the two or more components were previously not in contact with one another, but through combining, the components come into contact with one another. Examples of components that may be combined include, but are not limited to, lactose, magnesium stearate, API, excipient preblend (i.e., lactose:MgSt mixture), and API preblend (i.e., API:MgSt mixture).

As used herein, the term “processing” or “processed” is used to refer to methods of particle size reduction and formulating including, but not limited to, blending, high-shear blending, milling, mechanofusion, mixing, and micronization methods such jet milling and ball milling. Examples of processing methods are described herein. In an embodiment, processing includes any significant handling of the preblends or the formulation. In an embodiment, significant handling of the preblends or the formulation includes blending the preblends or the formulation.

As used herein, the terms “resting,” “rested,” and “resting period” are used to refer to the methods of formulating whereby said formulation is idled and is thus not subjected to any further movement, processing, or any formulation procedures that would agitate the formulation during the specified period. It should be recognized that a purpose of the resting period is to allow any residual energy within the formulation to dissipate. In some embodiments, the residual energy within the formulation is static charge.

As used herein, the term “co-milling” or “co-milled” is used to refer to a range of powder processing methods used to break up agglomerates including, but not limited to, screen mills that can be various shapes including conical. In an embodiment, co-milling refers to screen milling.

As used herein, the terms “blend,” “blending,” and “blended” refer to mixing multiple components to obtain a formulation. The resulting blended formulation can be homogeneous. The term “blend” is also used herein to describe the product that results from blending. In this context, “blend” is synonymous with “formulation.”

The present disclosure also includes salt forms of the compounds described herein. Examples of salts (or salt forms) include, but are not limited to, mineral or organic acid salts of basic residues such as amines, alkali or organic salts of acidic residues such as carboxylic acids, and the like. Lists of suitable salts are found in Remington's Pharmaceutical Sciences, 17^{th} ed., Mack Publishing Company, Easton, Pa., 1985, p. 1418, the disclosure of which is hereby incorporated by reference in its entirety.

As used herein, the term “AUC_{0-∞}” refers to the total area under the plasma concentration-time curve extrapolated to an infinite time. Values of AUC_{0-∞} are reported in units of h·pg/mL or hours-picograms/milliliters.

As used herein, the term “AUC_{0-last non-zero}” refers to the total area under the plasma concentration-time curve from dosing (time 0) to the time of the last measurable (non-zero) concentration. Values of AUC_{0-last non-zero} are reported in units of h·pg/mL or hours-picograms/milliliters.

As used herein, the term “AUC_{over dosing interval}” refers to the total area under the plasma concentration-time curve over a 10-day dosing interval. Values of AUC_{over dosing interval} are reported in units of h·pg/mL or hours-picograms/milliliters.

As used herein, the term “AUC_{0-12 hours}” refers to the total area under the plasma concentration-time curve from dosing (time 0) to 12 hours post-dose (time 12). Values of AUC_{0-12 hours} are reported in units of h·pg/mL or hours-picograms/milliliters.

As used herein, the term “AUC_{0-24 hours}” refers to the total area under the plasma concentration-time curve from dosing (time 0) to 24 hours post-dose (time 24). Values of AUC_{0-24 hours} are reported in units of h·pg/mL or hours-picograms/milliliters.

As used herein, the term “C_{trough}” refers to the trough concentration of Compound I in the blood plasma prior to administration of a subsequent dose. Values of C_{trough} are reported in units of pg/mL or picograms/milliliters.

As used herein, the term “Cₘₐₓ” refers to the maximum concentration of Compound I in the blood plasma prior to administration of a subsequent dose. Values of Cₘₐₓ are reported in units of pg/mL or picograms/milliliters.

As used herein, the term “percent change” is used to quantify the change in particular patient metrics, including, for example, fractional exhaled nitric oxide (FeNO), after administration of a pharmaceutical formulation. Percent change is calculated using the patient's baseline (Day 1 pre-dose) FeNO concentration (Xᵢ) and the patient's FeNO concentration after administration of a formulation comprising Compound I (X_{f}) according to the following formula:

$$( \frac{{X}_{i} - {X}_{f}}{{X}_{i}} ) \times 100$$

It Is to be understood that a negative (−) percent change indicates a reduction in the patient's FeNO upon administration of a formulation comprising Compound I. It should also be understood that the phrase “less than or equal to” in reference to a negative value, e.g., percent change, includes values that are more negative than the referenced value. For example, the phrase “wherein the subject's placebo-corrected percent change in FeNO concentration is less than or equal to −10.0%” includes, and is not limited to, percent changes such as −10.1%, −10.5%, and −11.0%. Reductions in FeNO concentration are consistent with a reduction in airway inflammation on administration of an API.

### Pharmaceutical Formulations

As discussed in WO 2011/051452, (S)-3-(3-(1-methyl-2-oxo-5-(pyrazolo[1,5-a]pyridin-3-yl)-1,2-dihydro-3H-imidazo[4,5-b]pyridin-3-yl)piperidin-1-yl)-3-oxopropanenitrile (Compound I) is a potent JAK2 and JAK3 inhibitor that has clinical potential in the treatment of respiratory indications such as asthma and COPD. This compound has the following formula (see also WO 2016/124464), and is also referred to herein as Compound I:

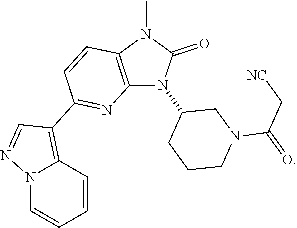

In some embodiments, the disclosure provides a pharmaceutical formulation comprising Compound I, or a pharmaceutically acceptable salt, solvate, hydrate, clathrate, or co-crystal thereof; one or more fillers; one or more glidants; and one or more lubricants.

The formulation can include Compound I in any suitable solid form, including amorphous, crystalline, or a combination thereof. For example, Compound I can exhibit any suitable crystalline form. Representative crystalline forms include one or more crystalline forms described in WO 2016/124464, which is incorporated herein in its entirety for all purposes.

In some embodiments, the disclosure provides a pharmaceutical formulation comprising Compound I:

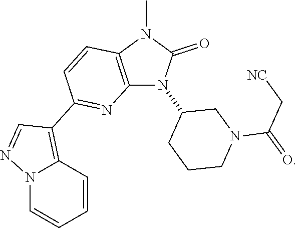

or a pharmaceutically acceptable salt thereof, lactose, and magnesium stearate.

In an embodiment, the pharmaceutical formulation comprises about 0.5 to about 11.0 weight percent Compound I, or a pharmaceutically acceptable salt thereof. In another embodiment, the pharmaceutical formulation further comprises about 0.5 to about 2.0 weight percent magnesium stearate. In yet another embodiment, the pharmaceutical formulation further comprises about 87.0 to about 99.0 weight percent lactose.

In an embodiment, the formulation comprises:
(a) 20.0-25.0 weight percent Compound I, or a pharmaceutically acceptable salt thereof;
(b) 0.5-5.0 weight percent magnesium stearate; and
(c) 75.0-80.0 weight percent lactose.

In another embodiment, the formulation comprises:
(a) 0.3-20.0 weight percent Compound I, or a pharmaceutically acceptable salt thereof;
(b) 0.3-10.0 weight percent magnesium stearate; and
(c) 70.0-99.4 weight percent lactose.

In another embodiment, the formulation comprises:
(a) 0.4-13.0 weight percent Compound I, or a pharmaceutically acceptable salt thereof;
(b) 0.4-7.0 weight percent magnesium stearate; and
(c) 80.0-89.0 weight percent lactose.

In still another embodiment, the pharmaceutical formulation comprises:
(a) about 0.5 to about 11.0 weight percent Compound I, or a pharmaceutically acceptable salt thereof;
(b) about 0.5 to about 2.0 weight percent magnesium stearate; and
(c) about 87.0 to about 99.0 weight percent lactose.

In yet another embodiment, the formulation comprises:
(a) about 1.1 weight percent Compound I, or a pharmaceutically acceptable salt thereof;
(b) about 1.0 weight percent magnesium stearate; and
(c) about 97.9 weight percent lactose.

In still another embodiment, the formulation comprises:
(a) about 10.3 weight percent Compound I, or a pharmaceutically acceptable salt thereof;
(b) about 1.2 weight percent magnesium stearate; and
(c) about 88.5 weight percent lactose.

In an embodiment, the formulation comprises:
(a) 1.0-5.0 mg of Compound I, or a pharmaceutically acceptable salt thereof;
(b) 0.1-1.0 mg of magnesium stearate; and
(c) 15.0-37.0 mg of lactose.

In another embodiment, the formulation comprises:
(a) 0.1-1.0 mg of Compound I;
(b) 0.1-1.0 mg of magnesium stearate; and
(c) 18.0-20.0 mg of lactose.

In yet another embodiment, the formulation comprises:
(a) about 0.2 mg of Compound I, or a pharmaceutically acceptable salt thereof;
(b) about 0.2 mg of magnesium stearate; and
(c) about 19.6 mg of lactose.

In still another embodiment, the formulation comprises:
(a) about 2.1 mg of Compound I, or a pharmaceutically acceptable salt thereof;
(b) about 0.3 mg of magnesium stearate; and
(c) about 17.7 mg of lactose.

In an embodiment, the formulation comprises:
(a) about 4.1 mg of Compound I, or a pharmaceutically acceptable salt thereof;
(b) about 0.5 mg of magnesium stearate; and
(c) about 35.4 mg of lactose.

In an embodiment, the formulation comprises 100 weight percent of Compound I, or a pharmaceutically acceptable salt thereof.

In another embodiment, the formulation comprises:
(a) a first preblend of (S)-3-(3-(1-methyl-2-oxo-5-(pyrazolo[1,5-a]pyridin-3-yl)-1,2-dihydro-3H-imidazo[4,5-b]pyridin-3-yl)piperidin-1-yl)-3-oxopropanenitrile:

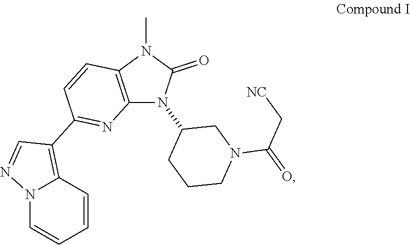

or a pharmaceutically acceptable salt thereof and magnesium stearate; and
(b) a second preblend of lactose and magnesium stearate.

In an embodiment, the first preblend is 1.0-15.0 weight percent magnesium stearate. In another embodiment, the first preblend is 1.0-11.0 weight percent magnesium stearate. In an embodiment, the first preblend is about 6.5 weight percent magnesium stearate.

In yet another embodiment, the first preblend is 1.0-11.0 weight percent magnesium stearate and the second preblend is 0.2-10.0 weight percent magnesium stearate. In still another embodiment, the first preblend is 1.0-11.0 weight percent magnesium stearate and the second preblend is 0.2-5.0 weight percent magnesium stearate. In an embodiment, the first preblend is 5.0-11.0 weight percent magnesium stearate and the second preblend is 0.2-10.0 weight percent magnesium stearate. In another embodiment, the first preblend is about 6.5 weight percent magnesium stearate and the second preblend is 0.2-10.0 weight percent magnesium stearate.

In another embodiment, the formulation is formulated as a capsule or a blister.

In an embodiment, the formulation is formulated as a capsule.

In yet another embodiment, the formulation comprises a bulk powder that is prepared without the use of plastic powder handling materials.

In still another embodiment, the formulation comprises a capsule that is prepared without the use of plastic powder handling materials.

In an embodiment, the formulation is prepared without plastic powder handling materials such as plastic spatulas, plastic scoops, or plastic transfer bags.

In another embodiment, the formulation is prepared without the use of a plastic transfer bag, such as a ChargeBag®.

In yet another embodiment, the formulation is prepared with stainless-steel powder handling materials.

In still another embodiment, the formulation is prepared with stainless-steel powder handling materials such as stainless-steel spatulas, stainless-steel scoops, stainless-steel funnels, or stainless-steel containers.

In some embodiments, the formulation is formulated as a capsule or a bulk powder.

In some embodiments, the formulation is formulated as a capsule. In some embodiments, the formulation is formulated as a bulk powder.

In some embodiments, the formulation is formulated as a capsule or blister. In some embodiments the formulation is formulated as a blister.

In some embodiments, the formulation comprises about 0.5 wt % to about 11.0 wt % of Compound I, or a pharmaceutically acceptable salt thereof. In some embodiments, the formulation comprises about 0.9 wt % to about 1.3 wt % of Compound I, or a pharmaceutically acceptable salt thereof. In some embodiments, the formulation comprises about 9.5 wt % to about 10.8 wt % of Compound I, or a pharmaceutically acceptable salt thereof. In some embodiments, the formulation comprises about 1.1 wt % of Compound I, or a pharmaceutically acceptable salt thereof. In some embodiments, the formulation comprises about 10.3 wt % of Compound I, or a pharmaceutically acceptable salt thereof.

In some embodiments, the formulation comprises about 87.0 wt % to about 99.0 wt % lactose. In some embodiments, the formulation comprises about 88.0 wt % to about 89.0 wt % lactose. In some embodiments, the formulation comprises about 97.0 wt % to about 98.0 wt % lactose. In some embodiments, the formulation comprises about 88.5 wt % lactose. In some embodiments, the formulation comprises 97.9 wt % lactose.

In some embodiments, the formulation comprises about 0.5 wt % to about 2.0 wt % magnesium stearate. In some embodiments, the formulation comprises about 0.8 wt % to about 1.4 wt % magnesium stearate. In some embodiments, the formulation comprises about 1.0 wt % magnesium stearate. In some embodiments, the formulation comprises about 1.2 wt % magnesium stearate.

In some embodiments, the formulation comprises about 0.5 wt % to about 11 wt % of Compound I, or a pharmaceutically acceptable salt thereof, about 87.0 wt % to about 99.0 wt % lactose, and about 0.5 wt % to about 2.0 wt % magnesium stearate.

In some embodiments, the formulation comprises about 1.1 wt % of Compound I, or a pharmaceutically acceptable salt thereof, about 97.9 wt % lactose, and about 1.0 wt % magnesium stearate.

In some embodiments, the formulation comprises about 10.3 wt % of Compound I, or a pharmaceutically acceptable salt thereof, about 88.5 wt % lactose, and about 1.2 wt % magnesium stearate.

Any suitable form of lactose can be used in the formulations described herein. Crystalline lactose, amorphous lactose, and mixtures thereof are suitable for use in the formulations of the disclosure. In some embodiments, the lactose is a spray-dried mixture of crystalline and amorphous lactose.

Any suitable form of magnesium stearate can be used in the formulations described herein. Several types and grades of magnesium stearate are suitable for use in the disclosed formulations. Accordingly, magnesium stearate having varying specific surface areas and varying median particle sizes can be used in the formulations disclosed herein.

In some embodiments, the formulation is formulated for oral administration via an inhaler (e.g., a tablet or capsule). In some embodiments, the formulation is contained within capsule. In some embodiments, the formulation is contained within a blister. In some embodiments the formulation is formulated as a bulk powder.

In some embodiments, the formulation is formulated such that Compound I is present in an amount ranging from about 0.1 mg to about 4.2 mg. In some embodiments, Compound I is present in an amount of about 0.2 mg, about 2.0 mg, or about 4.0 mg. In some embodiments, Compound I is present in an amount of about 0.2 mg. In some embodiments, Compound I is present in an amount of about 2.0 mg. In some embodiments, Compound I is present in an amount of about 4.0 mg.

In some embodiments, the formulation is formulated such that Compound I is present in an amount of about 0.15-0.25 mg. Accordingly, in an embodiment, the formulation comprises about 0.2 mg of Compound I, about 19.6 mg lactose, and about 0.2 mg magnesium stearate. In some embodiments, the formulation is formulated as a 0.2 mg strength tablet or capsule. In some embodiments, the formulation is formulated as a 0.2 mg strength capsule.

In some embodiments, the formulation is formulated such that Compound I is present in an amount of about 1.5-2.5 mg. Accordingly, in some embodiments, the formulation comprises about 2.0 mg of Compound I, about 17.7 mg lactose, and about 0.3 mg magnesium stearate. In some embodiments, the formulation is formulated as a 2.0 mg strength tablet or capsule. In some embodiments, the formulation is formulated as a 2.0 mg strength capsule.

In some embodiments, the formulation is formulated such that Compound I is present in an amount of about 3.5-4.5 mg. Accordingly, in some embodiments, the formulation comprises about 4.0 mg of Compound I, about 35.4 mg lactose, and about 0.5 mg magnesium stearate. In some embodiments, the formulation is formulated as a 4.0 mg strength tablet or capsule. In some embodiments, the formulation is formulated as a 4.0 mg strength capsule.

Additional fillers or diluents (i.e., pharmaceutically acceptable excipients) for use in the formulations of the disclosure include fillers or diluents typically used in the formulation of pharmaceuticals. Examples of fillers or diluents for use in accordance with the disclosure include, but are not limited to, sugars such as lactose (e.g., anhydrous lactose, directly compressible anhydrous lactose, lactose monohydrate, modified lactose monohydrate), dextrose, glucose, sucrose, cellulose, starches and carbohydrate derivatives, polysaccharides (including dextrates and maltodextrin), polyols (including mannitol, xylitol, and sorbitol), cyclodextrins, calcium carbonates, magnesium carbonates, microcrystalline cellulose, combinations thereof, and the like. In some embodiments the filler or diluent is lactose, microcrystalline cellulose, or a combination thereof. In some embodiments the filler or diluent is trehalose.

The formulations of the disclosure can also comprise additional pharmaceutically acceptable excipients, including surfactants, polymers, and binders. Surfactants suitable for use in the formulations of the disclosure include surfactants commonly used in the formulation of pharmaceuticals. Examples of surfactants include, but are not limited to, ionic- and nonionic surfactants or wetting agents commonly used in the formulation of pharmaceuticals, such as ethoxylated castor oil, polyglycolyzed glycerides, acetylated monoglycerides, sorbitan fatty acid esters, poloxamers, polyoxyethylene sorbitan fatty acid esters, polyoxyethylene derivatives, monoglycerides or ethoxylated derivatives thereof, diglycerides or polyoxyethylene derivatives thereof, sodium docusate, sodium laurylsulfate, cholic acid or derivatives thereof, lecithins, phospholipids, combinations thereof, and the like.

Compound I is administered as a pharmaceutical formulation, typically with a pharmaceutically acceptable carrier or excipient. In one embodiment the disclosure relates to a pharmaceutical formulation of Compound I with one or more pharmaceutically acceptable excipients. Suitable compositions can be in the form of tablets, capsules, or inhalable compositions. In one embodiment the disclosure provides a capsule comprising Compound I. In one embodiment, the disclosure provides a capsule filled with a formulation comprising Compound I. In one embodiment, the disclosure provides a blister filled with a formulation comprising Compound I. In one embodiment the disclosure provides an inhalable composition comprising Compound I.

The formulation can be delivered via a dry powder inhaler (DPI) for the treatment of respiratory diseases. For administration by inhalation using a dry powder inhaler, Compound I can be administered as a dry powder formulation with one or more carrier substances. Suitable inhalation carriers are known in the art and in one embodiment include crystalline sugars such as monosaccharides or disaccharides. In one embodiment the carrier is lactose. Compound I can also be administered as a dry powder formulation without carrier substances.

Dry powder formulations of the disclosure may also have additional excipients such as force control agents. A force control agent is an additive which reduces the cohesion between the fine particles within the powder formulation. This promotes de-agglomeration when the powder is dispensed from the inhaler. Suitable force control agents such as magnesium stearate, are known in the art to enhance the stability of dry powder formulations. In one embodiment the force control agent is a metal stearate such as magnesium stearate.

The dry powder formulations of the disclosure can be administered using a unit dose dry powder inhaler or a multi-dose dry powder inhaler. In a non-limiting example, the dry powder formulations of the disclosure can be administered using various dry powder inhalers such as GyroHaler® or Miat® Monodose RS01 or a lever operated inhaler such as that disclosed in WO2009/092770. In another non-limiting example, the dry powder formulations of the disclosure can be administered using various open-inhale-close devices. In a further embodiment the disclosure provides a kit comprising an inhaler in combination with a formulation provided herein. In a further embodiment the disclosure provides a kit comprising a dry powder inhaler in combination with a pharmaceutical formulation provided herein. In an embodiment, the disclosure provides a kit comprising a Miat Monodose RS01 dry powder inhaler in combination with a pharmaceutical formulation provided herein.

In an embodiment, Compound I is a polymorph form having the following diffraction angles (2Theta) based on cupric Kα1:
at approximately 8.25°;
at approximately 13.25°;
at approximately 15.40°;
at approximately 17.65°; and
at approximately 25.39° (Form II).

In an aspect, provided herein is a compound that is Compound II:

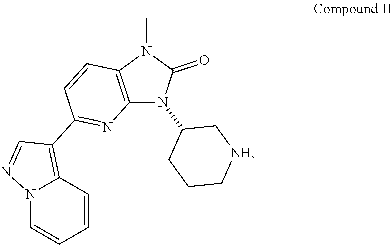

or a pharmaceutically acceptable solvate, hydrate, clathrate, or co-crystal thereof.

In another aspect, provided herein is a pharmaceutical formulation comprising Compound II, or a pharmaceutically acceptable solvate, hydrate, clathrate, or co-crystal thereof, and one or more pharmaceutically acceptable excipients.

I an embodiment, the pharmaceutical formulation comprises lactose and magnesium stearate.

In another embodiment, the formulation comprises:
(a) 20.0-25.0 weight percent Compound II, or a pharmaceutically acceptable solvate or hydrate thereof;
(b) 0.5-5.0 weight percent magnesium stearate; and
(c) 75.0-80.0 weight percent lactose.

In another embodiment, the formulation comprises:
(a) 0.3-20.0 weight percent Compound II, or a pharmaceutically acceptable solvate or hydrate thereof;
(b) 0.3-10.0 weight percent magnesium stearate; and
(c) 70.0-99.4 weight percent lactose.

In another embodiment, the formulation comprises:
(a) 0.4-13.0 weight percent Compound II, or a pharmaceutically acceptable solvate or hydrate thereof;
(b) 0.4-7.0 weight percent magnesium stearate; and
(c) 80.0-89.0 weight percent lactose.

In still another embodiment, the pharmaceutical formulation comprises:
(a) about 0.5 to about 11.0 weight percent Compound II, or a pharmaceutically acceptable solvate or hydrate thereof;
(b) about 0.5 to about 2.0 weight percent magnesium stearate; and
(c) about 87.0 to about 99.0 weight percent lactose.

In yet another embodiment, the formulation comprises:
(a) about 1.1 weight percent Compound II, or a pharmaceutically acceptable solvate or hydrate thereof;
(b) about 1.0 weight percent magnesium stearate; and
(c) about 97.9 weight percent lactose.

In still another embodiment, the formulation comprises:
(a) about 10.3 weight percent Compound II, or a pharmaceutically acceptable solvate or hydrate thereof;
(b) about 1.2 weight percent magnesium stearate; and
(c) about 88.5 weight percent lactose.

### Polymorph Form II

In an embodiment of the methods of treatment specified herein, (S)-3-(3-(1-methyl-2-oxo-5-(pyrazolo[1,5-a]pyridin-3-yl)-1,2-dihydro-3H-imidazo[4,5-b]pyridin-3-yl)piperidin-1-yl)-3-oxopropanenitrile (Compound I) is polymorph Form II having X-ray powder diffraction peak data according to the following table:

| 20 (°) | Intensity | Rel. Intensity |
|---|---|---|
| 8.25 | 5933 | 100.0 |
| 10.80 | 1472 | 24.8 |
| 12.60 | 761 | 12.8 |
| 13.25 | 2310 | 38.9 |
| 15.40 | 3572 | 60.2 |
| 17.65 | 2549 | 43.0 |
| 18.35 | 420 | 7.1 |
| 18.80 | 1325 | 22.3 |
| 20.15 | 1081 | 18.2 |
| 20.70 | 1393 | 23.5 |
| 20.90 | 687 | 11.6 |
| 22.55 | 938 | 15.8 |
| 24.29 | 1301 | 21.9 |
| 24.74 | 480 | 8.1 |
| 25.39 | 2776 | 46.8 |
| 25.99 | 627 | 10.6 |
| 26.24 | 585 | 9.9 |
| 26.74 | 1481 | 25.0 |
| 27.34 | 451 | 7.6 |
| 32.99 | 542 | 9.1 |

In an embodiment, Compound I is a polymorph form having the following diffraction angles (2Theta) based on cupric Kα1:
at approximately 8.25°;
at approximately 13.25°;
at approximately 15.40°;
at approximately 17.65°; and
at approximately 25.39° (Form II).

This polymorph form and its corresponding methods of preparation are disclosed in U.S. Pat. No. 10,087,196, which is incorporated by reference herein in its entirety. Form II is further characterized by an endotherm onset at 239° C. as measured by differential scanning calorimetry.

### Methods of Use

Compound I has been shown to be a potent inhibitor of the JAK family of enzymes, specifically JAK1, JAK2, JAK3, and TYK2. For example, the biological activity of the compound can be found in WO 2011/051452, which is incorporated herein by reference in its entirety. Inhibition of the family of JAK enzymes could inhibit signaling of many key pro-inflammatory cytokines, chemokines, and cells. Further, JAK inhibition represents an opportunity to interrupt inflammatory pathways implicated in the pathogenesis of multiple conditions including conditions associated with eosinophilic and non-eosinophilic inflammation that include respiratory diseases such as asthma, and chronic obstructive pulmonary disease (COPD). Thus, the formulations of the disclosure are useful in the treatment of conditions associated with eosinophilic and non-eosinophilic inflammation as defined herein, and respiratory diseases such as asthma and chronic obstructive pulmonary disease.

Compound I, and formulations comprising Compound I, are useful for the treatment of asthma and COPD, as well as other inflammatory conditions associated with eosinophilic inflammation and non-eosinophilic inflammation.

Thus, provided herein is a method of treating a disease or disorder in a subject in need thereof, wherein the method comprises administering to the subject a JAK inhibitor or a pharmaceutical formulation comprising a JAK inhibitor, and wherein the disease or disorder is selected from asthma, COPD, Asthma-COPD Overlap Syndrome (ACOS), and chronic bronchitis, or a combination thereof. The treatment methods disclosed herein are, at least, useful in treating particular COPD patient populations as demonstrated in clinical trial studies.

In an embodiment, the JAK inhibitor is selected from a TYK2 inhibitor, a JAK1 inhibitor, a JAK2 inhibitor, a JAK3 inhibitor, and a pan-JAK inhibitor. In another embodiment, the JAK inhibitor is a pan-JAK inhibitor. In yet another embodiment, the JAK inhibitor is Compound I:

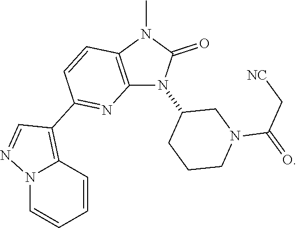

or a pharmaceutically acceptable salt thereof. In an embodiment, the subject is administered Compound I or a pharmaceutical formulation comprising Compound I or a pharmaceutical salt thereof and a pharmaceutically acceptable excipient. In yet another embodiment, the pharmaceutical formulation comprises magnesium stearate. In still another embodiment, the pharmaceutical formulation comprises lactose. In an embodiment, the subject is administered Compound I.

In an aspect, provided herein is a method of treating Asthma-COPD Overlap Syndrome (ACOS) in a subject in need thereof, wherein the method comprises administering to the subject Compound I, or a pharmaceutically acceptable salt thereof.

In another aspect, provided herein is a method of treating Asthma-COPD Overlap Syndrome (ACOS) in a subject in need thereof, wherein the method comprises administering to the subject a pharmaceutical formulation comprising Compound I, or a pharmaceutically acceptable salt thereof.

In an aspect, provided herein is a method of treating a disease or disorder in a subject in need thereof, wherein the method comprises administering to the subject Compound II, or a pharmaceutically acceptable solvate or hydrate thereof, wherein: the disease or disorder is selected from COPD, Asthma-COPD Overlap Syndrome (ACOS), and chronic bronchitis, or a combination thereof.

In another aspect, provided herein is a method of treating a disease or disorder in a subject in need thereof, wherein the method comprises administering to the subject a pharmaceutical formulation comprising Compound II, or a pharmaceutically acceptable solvate or hydrate thereof, wherein:
the disease or disorder is selected from COPD, Asthma-COPD Overlap Syndrome (ACOS), and chronic bronchitis, or a combination thereof.

In an embodiment, the disease or disorder is COPD.

In an aspect, provided herein is a method of treating a disease or disorder in a subject in need thereof, wherein the method comprises administering to the subject a therapeutically effective amount of Compound I, or a pharmaceutically acceptable salt thereof, wherein:
the disease or disorder is selected from asthma, COPD, Asthma-COPD Overlap Syndrome (ACOS), and chronic bronchitis, or a combination thereof; and
prior to administering the pharmaceutical formulation, the subject has a blood eosinophil level that is less than about 300 cells/μL.

In yet another aspect, provided herein is a method of treating a disease or disorder in a subject in need thereof, wherein the method comprises administering to the subject a pharmaceutical formulation comprising Compound I, or a pharmaceutically acceptable salt thereof, wherein:
the disease or disorder is selected from asthma, COPD, Asthma-COPD Overlap Syndrome (ACOS), and chronic bronchitis, or a combination thereof; and
prior to administering the pharmaceutical formulation, the subject has a blood eosinophil level that is less than about 300 cells/μL.

In still another aspect, provided herein is a method of treating a disease or disorder in a subject in need thereof, wherein the method comprises administering to the subject a therapeutically effective amount of Compound II, or a pharmaceutically acceptable salt, solvate, or hydrate thereof, wherein:
the disease or disorder is selected from asthma, COPD, Asthma-COPD Overlap Syndrome (ACOS), and chronic bronchitis, or a combination thereof; and
prior to administering the pharmaceutical formulation, the subject has a blood eosinophil level that is less than about 300 cells/μL.

In an aspect, provided herein is a method of treating a disease or disorder in a subject in need thereof, wherein the method comprises administering to the subject a pharmaceutical formulation comprising Compound II, or a pharmaceutically acceptable salt, solvate, or hydrate thereof, wherein:
the disease or disorder is selected from asthma, COPD, Asthma-COPD Overlap Syndrome (ACOS), and chronic bronchitis, or a combination thereof; and
prior to administering the pharmaceutical formulation, the subject has a blood eosinophil level that is less than about 300 cells/μL.

In an embodiment, the disease or disorder is COPD.

In yet another embodiment, prior to administering the pharmaceutical formulation, the subject has a blood eosinophil level that is less than or equal to about 280 cells/μL. In another embodiment, prior to administering the pharmaceutical formulation, the subject has a blood eosinophil level that is less than or equal to about 260 cells/μL. In yet another embodiment, prior to administering the pharmaceutical formulation, the subject has a blood eosinophil level that is less than or equal to about 240 cells/μL. In still another embodiment, prior to administering the pharmaceutical formulation, the subject has a blood eosinophil level that is less than or equal to about 220 cells/μL.

In an embodiment, prior to administering the pharmaceutical formulation, the subject has a blood eosinophil level that is about 10 cells/μL to about 299 cells/μL. In another embodiment, prior to administering the pharmaceutical formulation, the subject has a blood eosinophil level that is about 30 cells/μL to about 299 cells/μL. In yet another embodiment, prior to administering the pharmaceutical formulation, the subject has a blood eosinophil level that is about 50 cells/μL to about 290 cells/μL. In still another embodiment, prior to administering the pharmaceutical formulation, the subject has a blood eosinophil level that is about 60 cells/μL to about 270 cells/μL.

In an embodiment, prior to administering the pharmaceutical formulation, the subject has a blood eosinophil level that is about 60 cells/μL to about 250 cells/μL. In another embodiment, prior to administering the pharmaceutical formulation, the subject has a blood eosinophil level that is about 60 cells/μL to about 230 cells/μL. In yet another embodiment, prior to administering the pharmaceutical formulation, the subject has a blood eosinophil level that is 60 cells/μL to 230 cells/μL. In still another embodiment, prior to administering the pharmaceutical formulation, the subject has a blood eosinophil level that is about 70 cells/μL to about 220 cells/μL.

In another aspect, provided herein is a method of treating a disease or disorder in a subject in need thereof, wherein the method comprises administering to the subject a pharmaceutical formulation comprising Compound I, or a pharmaceutically acceptable salt thereof, wherein:
the disease or disorder is selected from asthma, COPD, Asthma-COPD Overlap Syndrome (ACOS), and chronic bronchitis, or a combination thereof; and
prior to administering the pharmaceutical formulation, the subject does not have a blood eosinophil level that is above about 220 cells/μL.

In an embodiment, prior to administering the pharmaceutical formulation, the subject does not have a blood eosinophil level that is above about 250 cells/μL. In another embodiment, prior to administering the pharmaceutical formulation, the subject does not have a blood eosinophil level that is above about 270 cells/μL. In yet another embodiment, prior to administering the pharmaceutical formulation, the subject does not have a blood eosinophil level that is above about 299 cells/μL. In still another embodiment, prior to administering the pharmaceutical formulation, the subject does not have a blood eosinophil level that is above about 299 cells/μL.

In an embodiment, prior to administering the pharmaceutical formulation, the subject does not have a blood eosinophil level that is above 299 cells/μL. In another embodiment, prior to administering the pharmaceutical formulation, the subject does not have a blood eosinophil level that is above about 320 cells/μL. In yet another embodiment, prior to administering the pharmaceutical formulation, the subject does not have a blood eosinophil level that is above about 330 cells/μL.

In yet another aspect, provided herein is a method of treating a disease or disorder in a subject in need thereof, wherein the method comprises administering to the subject a pharmaceutical formulation comprising Compound I, or a pharmaceutically acceptable salt thereof, wherein:
the disease or disorder is selected from asthma, COPD, Asthma-COPD Overlap Syndrome (ACOS), and chronic bronchitis, or a combination thereof;
prior to administering the pharmaceutical formulation, the subject has a blood eosinophil level that is less than about 300 cells/μL; and
the pharmaceutical formulation comprises lactose and magnesium stearate.

In another aspect, provided herein is a method of treating a disease or disorder in a subject in need thereof, wherein the method comprises administering to the subject a pharmaceutical formulation comprising Compound I, or a pharmaceutically acceptable salt thereof, wherein:
the disease or disorder is selected from COPD and chronic bronchitis, or a combination thereof; and
prior to administering the pharmaceutical formulation, the subject has a blood eosinophil level that is less than about 300 cells/μL.

In an aspect, provided herein is a method of treating a disease or disorder in a subject in need thereof, wherein the method comprises administering to the subject a pharmaceutical formulation comprising Compound I, or a pharmaceutically acceptable salt thereof, wherein:
the disease or disorder is selected from COPD and chronic bronchitis, or a combination thereof;
prior to administering the pharmaceutical formulation, the subject has a blood eosinophil level that is less than about 300 cells/μL; and
the pharmaceutical formulation comprises lactose and magnesium stearate.

In another aspect, provided herein is a method of treating COPD in a subject in need thereof, wherein the method comprises administering to the subject a pharmaceutical formulation comprising Compound I, or a pharmaceutically acceptable salt thereof, wherein:
prior to administering the pharmaceutical formulation, the subject has a blood eosinophil level that is less than about 300 cells/μL.

In an aspect, provided herein is a method of treating COPD in a subject in need thereof, wherein the method comprises administering to the subject a pharmaceutical formulation comprising Compound I, or a pharmaceutically acceptable salt thereof, wherein:
prior to administering the pharmaceutical formulation, the subject has a blood eosinophil level that is less than about 300 cells/μL.

In an embodiment, prior to administering the pharmaceutical formulation, the subject has a blood eosinophil level that is less than or equal to about 280 cells/μL. In another embodiment, prior to administering the pharmaceutical formulation, the subject has a blood eosinophil level that is less than or equal to about 260 cells/μL. In yet another embodiment, prior to administering the pharmaceutical formulation, the subject has a blood eosinophil level that is less than or equal to about 240 cells/μL. In still another embodiment, prior to administering the pharmaceutical formulation, the subject has a blood eosinophil level that is less than or equal to about 220 cells/μL.

In an embodiment, prior to administering the pharmaceutical formulation, the subject has a blood eosinophil level that is about 10 cells/μL to about 299 cells/μL. In another embodiment, prior to administering the pharmaceutical formulation, the subject has a blood eosinophil level that is about 30 cells/μL to about 299 cells/μL. In yet another embodiment, prior to administering the pharmaceutical formulation, the subject has a blood eosinophil level that is about 50 cells/μL to about 290 cells/μL. In still another embodiment, prior to administering the pharmaceutical formulation, the subject has a blood eosinophil level that is about 60 cells/μL to about 270 cells/μL.

In an embodiment, prior to administering the pharmaceutical formulation, the subject has a blood eosinophil level that is about 60 cells/μL to about 250 cells/μL. In another embodiment, prior to administering the pharmaceutical formulation, the subject has a blood eosinophil level that is about 60 cells/μL to about 230 cells/μL. In yet another embodiment, prior to administering the pharmaceutical formulation, the subject has a blood eosinophil level that is 60 cells/μL to 230 cells/μL. In still another embodiment, prior to administering the pharmaceutical formulation, the subject has a blood eosinophil level that is about 70 cells/μL to about 220 cells/μL.

In another aspect, provided herein is a method of treating COPD in a subject in need thereof, wherein the method comprises administering to the subject a pharmaceutical formulation comprising Compound I, or a pharmaceutically acceptable salt thereof, wherein:
prior to administering the pharmaceutical formulation, the subject has a blood eosinophil level that is less than about 300 cells/μL; and
the pharmaceutical formulation comprises lactose and magnesium stearate.

In an aspect, provided herein is a method of treating COPD in a subject in need thereof, wherein the method comprises administering to the subject a pharmaceutical formulation comprising Compound I, or a pharmaceutically acceptable salt thereof, wherein: prior to administering the pharmaceutical formulation, the subject does not have a blood eosinophil level that is above about 220 cells/μL.

In an embodiment, prior to administering the pharmaceutical formulation, the subject does not have a blood eosinophil level that is above about 250 cells/μL. In another embodiment, prior to administering the pharmaceutical formulation, the subject does not have a blood eosinophil level that is above about 270 cells/μL. In yet another embodiment, prior to administering the pharmaceutical formulation, the subject does not have a blood eosinophil level that is above about 299 cells/μL. In still another embodiment, prior to administering the pharmaceutical formulation, the subject does not have a blood eosinophil level that is above about 299 cells/μL.

In an embodiment, prior to administering the pharmaceutical formulation, the subject does not have a blood eosinophil level that is above 299 cells/μL. In another embodiment, prior to administering the pharmaceutical formulation, the subject does not have a blood eosinophil level that is above about 320 cells/μL. In yet another embodiment, prior to administering the pharmaceutical formulation, the subject does not have a blood eosinophil level that is above about 330 cells/μL.

In another aspect, provided herein is a method of treating COPD in a subject in need thereof, wherein the method comprises administering to the subject a pharmaceutical formulation comprising Compound I, or a pharmaceutically acceptable salt thereof, wherein: prior to administering the pharmaceutical formulation, the subject does not have a blood eosinophil level that is above about 220 cells/μL; and the formulation comprises lactose and magnesium stearate.

In yet another aspect, provided herein is a method of treating a disease or disorder in a subject in need thereof, wherein the method comprises administering to the subject a pharmaceutical formulation comprising Compound I, or a pharmaceutically acceptable salt thereof, wherein:
the disease or disorder is selected from asthma, COPD, Asthma-COPD Overlap Syndrome (ACOS), and chronic bronchitis, or a combination thereof; and
prior to administering the pharmaceutical formulation, the subject has a blood eosinophil level that is less than or equal to 149 cells/μL.

In an embodiment, prior to administering the pharmaceutical formulation, the subject has a blood eosinophil level that is less than or equal to 119 cells/μL.

In an aspect, provided herein is a method of treating a disease or disorder in a subject in need thereof, wherein the method comprises administering to the subject a therapeutically effective amount of Compound I, or a pharmaceutically acceptable salt thereof, wherein:
the disease or disorder is selected from asthma, COPD, Asthma-COPD Overlap Syndrome (ACOS), and chronic bronchitis, or a combination thereof; and
prior to administering the pharmaceutical formulation, the subject has a blood eosinophil level that is less than or equal to 89 cells/μL.

In still another aspect, provided herein is a method of treating a disease or disorder in a subject in need thereof, wherein the method comprises administering to the subject a pharmaceutical formulation comprising Compound I, or a pharmaceutically acceptable salt thereof, wherein:
the disease or disorder is selected from asthma, COPD, Asthma-COPD Overlap Syndrome (ACOS), and chronic bronchitis, or a combination thereof; and
prior to administering the pharmaceutical formulation, the subject has a blood eosinophil level that is less than or equal to 89 cells/μL.

In an embodiment of any of the above aspects, the pharmaceutical formulation comprises lactose and magnesium stearate.

In an embodiment of any of the above aspects, prior to administering the pharmaceutical formulation, the subject has a blood eosinophil level that is less than or equal to about 280 cells/μL. In another embodiment, prior to administering the pharmaceutical formulation, the subject has a blood eosinophil level that is less than or equal to about 260 cells/μL. In yet another embodiment, prior to administering the pharmaceutical formulation, the subject has a blood eosinophil level that is less than or equal to about 240 cells/μL. In still another embodiment, prior to administering the pharmaceutical formulation, the subject has a blood eosinophil level that is less than or equal to about 220 cells/μL.

In an embodiment of any of the above aspects, prior to administering the pharmaceutical formulation, the subject has a blood eosinophil level that is about 10 cells/μL to about 299 cells/μL. In another embodiment, prior to administering the pharmaceutical formulation, the subject has a blood eosinophil level that is about 30 cells/μL to about 299 cells/μL. In yet another embodiment, prior to administering the pharmaceutical formulation, the subject has a blood eosinophil level that is about 50 cells/μL to about 290 cells/μL. In still another embodiment, prior to administering the pharmaceutical formulation, the subject has a blood eosinophil level that is about 60 cells/μL to about 270 cells/μL.

In an embodiment of any of the above aspects, prior to administering the pharmaceutical formulation, the subject has a blood eosinophil level that is about 60 cells/μL to about 250 cells/μL. In another embodiment, prior to administering the pharmaceutical formulation, the subject has a blood eosinophil level that is about 60 cells/μL to about 230 cells/μL. In yet another embodiment, prior to administering the pharmaceutical formulation, the subject has a blood eosinophil level that is 60 cells/μL to 230 cells/μL. In still another embodiment, prior to administering the pharmaceutical formulation, the subject has a blood eosinophil level that is about 70 cells/μL to about 220 cells/μL.

In an embodiment of any of the above aspects, prior to administering the pharmaceutical formulation, the subject does not have a blood eosinophil level that is above about 220 cells/μL. In yet another embodiment, prior to administering the pharmaceutical formulation, the subject does not have a blood eosinophil level that is above about 250 cells/μL. In another embodiment, prior to administering the pharmaceutical formulation, the subject does not have a blood eosinophil level that is above about 270 cells/μL. In yet another embodiment, prior to administering the pharmaceutical formulation, the subject does not have a blood eosinophil level that is above about 299 cells/μL. In still another embodiment, prior to administering the pharmaceutical formulation, the subject does not have a blood eosinophil level that is above about 299 cells/μL.

In an embodiment of any of the above aspects, prior to administering the pharmaceutical formulation, the subject does not have a blood eosinophil level that is above 299 cells/μL. In another embodiment, prior to administering the pharmaceutical formulation, the subject does not have a blood eosinophil level that is above about 320 cells/μL. In yet another embodiment, prior to administering the pharmaceutical formulation, the subject does not have a blood eosinophil level that is above about 330 cells/μL.

In an embodiment, prior to administering the pharmaceutical formulation, the subject is undergoing treatment with a background therapy selected from an inhaled corticosteroid, an inhaled long-acting beta-agonist, and an inhaled long-acting muscarinic antagonist, or a combination of two or more thereof. In an embodiment, the subject is treatment naïve.

In still another embodiment, the disease or disorder is COPD. In an embodiment, the disease or disorder is chronic bronchitis. In another embodiment, the disease or disorder is ACOS.

In yet another embodiment, the subject is diagnosed with non-eosinophilic inflammation. In still another embodiment, the subject is diagnosed with eosinophilic inflammation. In an embodiment, the subject is further diagnosed with non-eosinophilic COPD. In another embodiment, the non-eosinophilic COPD is characterized by a blood eosinophil level that is less than about 300 cells/μL (e.g., less than 300 cells/μL or 0 cells/μL to 299 cells/μL). In yet another embodiment, the non-eosinophilic COPD is characterized by a T2 low phenotype. In another embodiment, the subject is further diagnosed with eosinophilic COPD. In yet another embodiment, the eosinophilic COPD is characterized by a blood eosinophil level that is greater than or equal to about 300 cells/μL (e.g., greater than or equal to 300 cells/μL; 300 cells/μL to 500 cells/μL; 300 cells/μL to 1,000 cells/μL; or 300 cells/μL to 1,500 cells/μL). In still another embodiment, the eosinophilic COPD is characterized by a T2 high phenotype. In another embodiment, the eosinophilic COPD is characterized by a blood eosinophil level that is greater than or equal to about 150 cells/μL. In yet another embodiment, the subject is diagnosed with exercise intolerance. In still another embodiment, the subject is diagnosed with dyspnea.

The modified Medical Research Council (mMRC) dyspnea questionnaire is frequently used in COPD as breathlessness is a crucial symptom in this condition (Respir. Med. 2007, 101, 399-410). The mMRC version now used consists of scale ranges from grade 0 to 4. It is important to note that it does not measure breathlessness directly, unlike other scales such as the Borg scale. Rather it measures the degree of activity at which a person gets breathlessness (such as “with strenuous exercise”) or limits what a person can do (such as “too breathless to leave the house”). It consists of five grades (0 to 4) that contain statements describing a range of physical limitations associated with breathlessness.

There is an assumption that the mMRC Grades are Guttman scaled (Br. J. Clin. Psychol. 2007, 46, 1-18) in which a person who fulfils the criteria for Grade 4, should also fulfil the criteria for Grade 3, 2 etc. Except for mMRC Grade 0 (“not troubled by breathlessness except on strenuous exercise”), each grade consists of two different activity descriptions. For example, the components of Grade 4 include “too breathless to leave the house” or “breathless when dressing.”

The mMRC breathlessness scale has good discriminative ability and is a simple method of categorizing patients with COPD in terms of their disability (Thorax 1999, 54, 581-586 and Int. J. Tuberc. Lung Dis. 1999, 3, 920-926) and survival (Resp. Med. 2008, 102, Suppl. 1, S27-S35). Thus, it is recommended for use as a marker of disability in international COPD guidelines (National Clinical Guideline Centre (UK). Chronic Obstructive Pulmonary Disease: Management of Chronic Obstructive Pulmonary Disease in Adults in Primary and Secondary Care. London: Royal College of Physicians (UK); 2010 Jun. and Can. Respir. J. 2007, 14, Suppl. B, 5B-32B) and used to assess suitability for pulmonary rehabilitation in the UK (Can. Respir. J. 2007, 14, Suppl. B, 5B-32B).

In an embodiment, prior to administering the pharmaceutical formulation, the subject's dyspnea according to the modified Medical Research Council (mMRC) scale is greater than or equal to 0. In another embodiment, prior to administering the pharmaceutical formulation, the subject's dyspnea according to the mMRC scale is greater than or equal to 1. In yet another embodiment, prior to administering the pharmaceutical formulation, the subject's dyspnea according to the mMRC scale is greater than or equal to 2. In still another embodiment, prior to administering the pharmaceutical formulation, the subject's dyspnea according to the mMRC scale is greater than or equal to 3. In an embodiment, prior to administering the pharmaceutical formulation, the subject's dyspnea according to the mMRC scale is 4.

The COPD assessment test (CAT) is an easy and simple measurement with an eight-item questionnaire including severity of cough, sputum, chest tightness, dyspnea, activities, confidence, sleep, and energy (Prim. Care Respir. J. 2009, 18, 208-215; Eur. Respir. J. 2009, 34, 648-654; and Eur. Respir. J. 2011, 38, 29-35). Item scores range from 0 to 5 points resulting in a total CAT score ranging from 0 to 40 points (Respir. J. 2009, 34, 648-654). Higher scores denote a more severe impact of COPD on a patient's life.

It is widely used in daily practice to assess and quantify the impacts of COPD symptoms on the health status, and correlates well with the SGRQ score in clinically stable COPD patients (Eur. Respir. J. 2011, 38, 29-35). It is one of the key determinants in assessing disease severity and guiding treatment in Global Initiative for Chronic Obstructive Lung Disease (GOLD) report (Global Initiative for Chronic Obstructive Lung Disease, Global strategy for the diagnosis, management and prevention of chronic obstructive pulmonary disease, Report, 2020). Furthermore, a significant increase in CAT total score at a clinic visit is useful to detect worsening or exacerbation of COPD (Respiration, 2012, 84, 193-199 and Int. J. Chron. Obstruct. Pulmon. Dis. 2015, 10, 277-282).

In an embodiment, prior to administering the pharmaceutical formulation, the subject has a COPD assessment test (CAT) score of 4 to 30. In another embodiment, prior to administering the pharmaceutical formulation, the subject has a CAT score of less than 10. In yet another embodiment, prior to administering the pharmaceutical formulation, the subject has a CAT score of greater than or equal to 4. In still another embodiment, prior to administering the pharmaceutical formulation, the subject has a CAT score of greater than or equal to 8.

In an embodiment, prior to administering the pharmaceutical formulation, the subject has a CAT score of greater than or equal to 10. In another embodiment, prior to administering the pharmaceutical formulation, the subject has a CAT score of greater than or equal to 12. In yet another embodiment, prior to administering the pharmaceutical formulation, the subject has a CAT score of greater than or equal to 16. In still another embodiment, prior to administering the pharmaceutical formulation, the subject has a CAT score of greater than or equal to 20.

In an embodiment, prior to administering the pharmaceutical formulation, the subject has a CAT score of greater than or equal to 24. In another embodiment, prior to administering the pharmaceutical formulation, the subject has a CAT score of greater than or equal to 28. In yet another embodiment, prior to administering the pharmaceutical formulation, the subject has a CAT score of greater than or equal to 30. In still another embodiment, prior to administering the pharmaceutical formulation, the subject has an average CAT score of about 15.

The standard respiratory function test for case detection of chronic obstructive pulmonary disease (COPD) is spirometry. A key measurement in spirometry is forced vital capacity (FVC), which is a measurement of the total amount of air exhaled during the lung function test. It is commonly used to assess lung function and helps differentiate between obstructive and restrictive lung diseases.

The criterion for COPD diagnosis defined in guidelines is based on the FEV₁/FVC ratio, or forced expiratory ratio (FER), and its severity is based on forced expiratory volume in one second (FEV₁) from measurements obtained during maximal forced expiratory maneuvers, where FVC is Forced Vital Capacity. Spirometry is a safe and practical procedure, and when conducted by a trained operator using a spirometer that provides quality feedback, the majority of patients can be coached to provide acceptable and repeatable results. This allows potentially wide application of testing to improve recognition and diagnosis of COPD, such as for case finding in primary care.

Spirometry is accepted as the diagnostic test to assess airflow obstruction and classify severity of disease, based on specific cut points for FER (FEV₁/FVC <0.7 after bronchodilator) and FEV₁. Specifically, FEV₁/FVC cut points are defined as follows: mild 0.8 predicted, moderate 0.5-0.8, severe 0.3-0.49 predicted, and very severe <0.3 predicted (Global Initiative for Chronic Obstructive Lung Disease (GOLD), Global Strategy for the Diagnosis, Management and Prevention of COPD, 2013). Traditionally, an FEV₁/FVC of 0.7 has been considered normal and a post-bronchodilator FEV₁/FVC <0.7 is required for confirmation as having COPD (GOLD 2025).

FEV₁ normally decreases with age, and the rate of fall is an important spirometric indicator of disease progression in COPD. In healthy, non-smoking adults the decrease is about 30 mL/year with an upper limit of about 50 mL/year (Am. Rev. Respir. Dis. 1988, 138, 837-849; Am. Rev. Respir. Dis. 1992, 146, 855-859; and Thorax 1997, 52, 820-827). A decrease greater than this is considered abnormally rapid.

Maximum flow achieved during forced expiration decreases progressively as lung volume falls and is most evident in the expiratory flow-volume curve where flow is plotted as a function of volume. Although flow and volume are complex biological signals, the curve is highly repeatable in both healthy and obstructed individuals and the shape of the curve can be helpful as it reflects the underlying mechanics limiting maximal flow. In healthy younger adults, the shape of the flow-volume curve usually approximates a straight-sided triangle with maximum flows decreasing linearly with lung volume. In people with obstructive lung disease, key physiologic features of the flow-volume curve are reduced expiratory flows in proportion to disease severity and the presence of a concavity in the descending limb; the latter indicating an abnormal decrease in maximal flow as lung volume falls.

The forced expiratory flow between 25% and 75% (FEF₂₅₋₇₅) is a measurement of airflow during spirometry, specifically the average flow rate during the middle half of a forced exhalation (from 25% to 75% of the forced vital capacity). It's often used as an indicator of small airway function, and reduced FEF₂₅₋₇₅ can suggest airway obstruction, particularly in the smaller airways.

In an embodiment, prior to administering the pharmaceutical formulation, the subject's forced expiratory volume in one second (FEV₁) is less than or equal to about 80% of predicted normal (e.g., about 30% to about 80%; about 40% to about 80%; about 42% to about 77%; and about 34% to about 77%). In another embodiment, prior to administering the pharmaceutical formulation, the subject's FEV₁ is less than or equal to about 70% of predicted normal (e.g., about 40% to about 70% and about 30% to about 70%). In yet another embodiment, prior to administering the pharmaceutical formulation, the subject's FEV₁ is less than or equal to about 60% of predicted normal (e.g., about 30% to about 60%; about 59.9%; about 60.1%; and about 59.9% to about 60.1%). In still another embodiment, prior to administering the pharmaceutical formulation, the subject's FEV₁ is less than or equal to about 50% of predicted normal (e.g., about 30% to about 50%; about 34% to about 50%; and about 42% to about 50%).

In an embodiment, prior to administering the pharmaceutical formulation, the subject's pre-bronchodilator forced expiratory volume in one second (FEV₁) is less than or equal to about 80% of predicted normal. In another embodiment, prior to administering the pharmaceutical formulation, the subject's pre-bronchodilator FEV₁ is less than or equal to about 70% of predicted normal. In yet another embodiment, prior to administering the pharmaceutical formulation, the subject's pre-bronchodilator FEV₁ is less than or equal to about 60% of predicted normal. In still another embodiment, prior to administering the pharmaceutical formulation, the subject's pre-bronchodilator FEV₁ is less than or equal to about 50% of predicted normal.

In an embodiment, prior to administering the pharmaceutical formulation, the subject's FEV₁ is less than or equal to about 35% of predicted normal. In yet another embodiment, prior to administering the pharmaceutical formulation, the subject's FEV₁ is less than or equal to about 40% of predicted normal. In another embodiment, prior to administering the pharmaceutical formulation, the subject's average FEV₁ is about 60% of predicted normal (e.g., about 59.9%; about 60.1%; and about 59.9% to about 60.1%). In yet another embodiment, prior to administering the pharmaceutical formulation, the subject's FEV₁ is about 30% to about 80% of predicted normal (e.g., about 30% to about 80%; about 40% to about 80%; about 42% to about 77%; and about 34% to about 77%). In yet another embodiment, prior to administering the pharmaceutical formulation, the subject's FEV₁ is less than or equal to about 2.5 L (e.g., about 0.8 L to about 2.5 L; about 0.9 L to about 2.5 L; about 0.86 L to about 2.41 L; about 1.69 L; and about 1.7 L). In still another embodiment, prior to administering the pharmaceutical formulation, the subject's FEV₁ is less than or equal to about 2.0 L (e.g., about 0.8 L to about 2.0 L; about 0.9 L to about 2.0 L; about 1.69 L; and about 1.7 L). In an embodiment, prior to administering the pharmaceutical formulation, the subject's FEV₁ is about 0.8 L to about 2.5 L (e.g. about 0.86 L to about 2.41 L; about 0.9 L to about 2.4 L; about 1.69 L; and about 1.7 L).

In an embodiment, prior to administering the pharmaceutical formulation, the subject's pre-bronchodilator FEV₁ is less than or equal to about 35% of predicted normal. In an embodiment, prior to administering the pharmaceutical formulation, the subject's pre-bronchodilator FEV₁ is less than or equal to about 40% of predicted normal. In another embodiment, prior to administering the pharmaceutical formulation, the subject's average pre-bronchodilator FEV₁ is about 60% of predicted normal. In yet another embodiment, prior to administering the pharmaceutical formulation, the subject's pre-bronchodilator FEV₁ is about 30% to about 80% of predicted normal (e.g., about 40% to about 80% and about 42% to about 77%). In yet another embodiment, prior to administering the pharmaceutical formulation, the subject's pre-bronchodilator FEV₁ is less than or equal to about 2.5 L (e.g., about 0.8 L to about 2.5 L; about 0.9 L to about 2.5 L; about 0.86 L to about 2.41 L; about 1.69 L; and about 1.7 L). In still another embodiment, prior to administering the pharmaceutical formulation, the subject's pre-bronchodilator FEV₁ is less than or equal to about 2.0 L (e.g., about 0.8 L to about 2.0 L; about 0.9 L to about 2.0 L; about 1.69 L; and about 1.7 L). In an embodiment, prior to administering the pharmaceutical formulation, the subject's pre-bronchodilator FEV₁ is about 0.8 L to about 3.2 L (e.g., about 1.1% to about 3.0% and about 2.0%). In another embodiment, prior to administering the pharmaceutical formulation, the subject's pre-bronchodilator FEV₁ is about 0.8 L to about 2.5 L (e.g. about 0.86 L to about 2.41 L; about 0.9 L to about 2.4 L; about 1.69 L; and about 1.7 L).

In an embodiment, prior to administering the pharmaceutical formulation, the subject's FEV₁ is less than or equal to about 1.5 L (e.g., about 0.8 L to about 0.9 L). In another embodiment, prior to administering the pharmaceutical formulation, the subject's FEV₁ is less than or equal to about 1.0 L (e.g., about 0.8 L to about 0.9 L). In yet another embodiment, prior to administering the pharmaceutical formulation, the subject's FEV₁ is less than or equal to about 0.9 L (e.g., 0.8 L to about 0.9 L). In still another embodiment, prior to administering the pharmaceutical formulation, the subject's FEV₁ is less than or equal to about 0.8 L (e.g., about 0.86 L). In an embodiment, prior to administering the pharmaceutical formulation, the subject's average FEV₁ is about 1.7 L (e.g., 1.69 L).

In an embodiment, prior to administering the pharmaceutical formulation, the subject's pre-bronchodilator FEV₁ is less than or equal to about 1.5 L. In another embodiment, prior to administering the pharmaceutical formulation, the subject's pre-bronchodilator FEV₁ is less than or equal to about 1.0 L. In yet another embodiment, prior to administering the pharmaceutical formulation, the subject's pre-bronchodilator FEV₁ is less than or equal to about 0.9 L. In still another embodiment, prior to administering the pharmaceutical formulation, the subject's pre-bronchodilator FEV₁ is less than or equal to about 0.8 L. In an embodiment, prior to administering the pharmaceutical formulation, the subject's average pre-bronchodilator FEV₁ is about 1.7 L.

In an embodiment, prior to administering the pharmaceutical formulation, the subject's FEV₁ is less than or equal to about 90% of predicted normal (e.g., about 50% to about 90%; about 52% to about 86%; and about 69.8%). In another embodiment, prior to administering the pharmaceutical formulation, the subject's FEV₁ is less than or equal to about 88% of predicted normal (e.g., about 50% to about 90%; about 52% to about 86%; and about 69.8%). In an embodiment, prior to administering the pharmaceutical formulation, the subject's FEV₁ is less than or equal to about 80% of predicted normal (e.g., about 52% to about 80% and about 69.8%). In yet another embodiment, prior to administering the pharmaceutical formulation, the subject's FEV₁ is less than or equal to about 70% of predicted normal (e.g., about 52% to about 70% and about 69.8%). In still another embodiment, prior to administering the pharmaceutical formulation, the subject's FEV₁ is less than or equal to about 60% of predicted normal (e.g., about 52% to about 60%).

In an embodiment, prior to administering the pharmaceutical formulation, the subject's post-bronchodilator FEV₁ is less than or equal to about 90% of predicted normal. In another embodiment, prior to administering the pharmaceutical formulation, the subject's post-bronchodilator FEV₁ is less than or equal to about 88% of predicted normal. In an embodiment, prior to administering the pharmaceutical formulation, the subject's post-bronchodilator FEV₁ is less than or equal to about 80% of predicted normal. In yet another embodiment, prior to administering the pharmaceutical formulation, the subject's post-bronchodilator FEV₁ is less than or equal to about 70% of predicted normal. In still another embodiment, prior to administering the pharmaceutical formulation, the subject's post-bronchodilator FEV₁ is less than or equal to about 60% of predicted normal.

In an embodiment, prior to administering the pharmaceutical formulation, the subject's FEV₁ is less than or equal to about 50% of predicted normal (e.g., about 40% to about 50%). In another embodiment, prior to administering the pharmaceutical formulation, the subject's FEV₁ is less than or equal to about 40% of predicted normal (e.g., about 30% to about 40%). In yet another embodiment, prior to administering the pharmaceutical formulation, the subject's average FEV₁ is about 70% of predicted normal (e.g., about 69.8%). In an embodiment, prior to administering the pharmaceutical formulation, the subject's FEV₁ is about 50% to about 90% of predicted normal (e.g., about 52% to about 86%). In another embodiment, prior to administering the pharmaceutical formulation, the subject's FEV₁ is about 50% to about 88% of predicted normal (e.g., about 52% to about 86%).

In an embodiment, prior to administering the pharmaceutical formulation, the subject's post-bronchodilator FEV₁ is less than or equal to about 50% of predicted normal. In another embodiment, prior to administering the pharmaceutical formulation, the subject's post-bronchodilator FEV₁ is less than or equal to about 40% of predicted normal. In yet another embodiment, prior to administering the pharmaceutical formulation, the subject's average post-bronchodilator FEV₁ is about 70% of predicted normal. In an embodiment, prior to administering the pharmaceutical formulation, the subject's post-bronchodilator FEV₁ is about 50% to about 90% of predicted normal. In yet another embodiment, prior to administering the pharmaceutical formulation, the subject's post-bronchodilator FEV₁ is about 50% to about 88% of predicted normal.

In still another embodiment, prior to administering the pharmaceutical formulation, the subject's FEV₁ is less than or equal to about 3.0 L (e.g., about 1.12 L to about 2.98 L; about 1.1 L to about 3.0 L; about 1.98 L; and about 2.0 L). In an embodiment, prior to administering the pharmaceutical formulation, the subject's FEV₁ is less than or equal to about 2.5 L (e.g., about 1.12 L to about 2.5 L and about 1.98 L). In yet another embodiment, prior to administering the pharmaceutical formulation, the subject's FEV₁ is less than or equal to about 2.0 L (e.g., about 1.12 L to about 2.0 L and about 1.98 L). In still another embodiment, prior to administering the pharmaceutical formulation, the subject's FEV₁ is less than or equal to about 1.5 L (e.g., about 1.12 L to about 1.5 L).

In still another embodiment, prior to administering the pharmaceutical formulation, the subject's post-bronchodilator FEV₁ is less than or equal to about 3.0 L. In an embodiment, prior to administering the pharmaceutical formulation, the subject's post-bronchodilator FEV₁ is less than or equal to about 2.5 L. In yet another embodiment, prior to administering the pharmaceutical formulation, the subject's post-bronchodilator FEV₁ is less than or equal to about 2.0 L. In still another embodiment, prior to administering the pharmaceutical formulation, the subject's post-bronchodilator FEV₁ is less than or equal to about 1.5 L.

In an embodiment, prior to administering the pharmaceutical formulation, the subject's FEV₁ is less than or equal to about 1.2 L (e.g., 1.12 L to about 1.2 L). In another embodiment, prior to administering the pharmaceutical formulation, the subject's FEV₁ is less than or equal to about 1.1 L. In yet another embodiment, prior to administering the pharmaceutical formulation, the subject's FEV₁ is less than or equal to about 1.0 L. In still another embodiment, prior to administering the pharmaceutical formulation, the subject's average FEV₁ is about 2.0 L (e.g., 1.98 L or 2.0 L). In an embodiment, prior to administering the pharmaceutical formulation, the subject's FEV₁ is about 0.8 L to about 3.1 L (e.g., about 1.1 L to about 3.0 L).

In an embodiment, prior to administering the pharmaceutical formulation, the subject's post-bronchodilator FEV₁ is less than or equal to about 1.2 L. In another embodiment, prior to administering the pharmaceutical formulation, the subject's post-bronchodilator FEV₁ is less than or equal to about 1.1 L. In yet another embodiment, prior to administering the pharmaceutical formulation, the subject's post-bronchodilator FEV₁ is less than or equal to about 1.0 L. In still another embodiment, prior to administering the pharmaceutical formulation, the subject's average post-bronchodilator FEV₁ is about 2.0 L. In an embodiment, prior to administering the pharmaceutical formulation, the subject's post-bronchodilator FEV₁ is about 1.0 L to about 3.1 L.

In another embodiment, prior to administering the pharmaceutical formulation, the subject's forced vital capacity (FVC) is less than or equal to about 6.0 L (e.g., about 2.1 L to about 4.3 L; about 2.4 L to about 5.1 L; about 3.3 L; and about 3.6 L). In yet another embodiment, prior to administering the pharmaceutical formulation, the subject's forced vital capacity (FVC) is less than or equal to about 5.5 L (e.g., about 2.1 L to about 4.3 L; about 2.4 L to about 5.1 L; about 3.3 L; and about 3.6 L). In still another embodiment, prior to administering the pharmaceutical formulation, the subject's FVC is less than or equal to about 5.1 L (e.g., about 2.1 L to about 4.3 L; about 2.4 L to about 5.1 L; about 3.3 L; and about 3.6 L). In an embodiment, prior to administering the pharmaceutical formulation, the subject's FVC is less than or equal to about 4.5 L (e.g., about 2.1 L to about 4.3 L; about 3.3 L; and about 3.6 L). In another embodiment, prior to administering the pharmaceutical formulation, the subject's FVC is less than or equal to about 4.3 L (e.g., about 2.1 L to about 4.3 L; about 3.3 L; and about 3.6 L). In yet another embodiment, prior to administering the pharmaceutical formulation, the subject's average FVC is about 3.6 L (e.g., 3.3 L or 3.6 L). In still another embodiment, prior to administering the pharmaceutical formulation, the subject's average FVC is about 3.3 L (e.g., 3.3 L or 3.6 L). In an embodiment, prior to administering the pharmaceutical formulation, the subject's FVC is about 2.0 L to about 4.5 L (e.g., about 2.1 L to about 4.3 L; 3.3 L; or 3.6 L). In yet another embodiment, prior to administering the pharmaceutical formulation, the subject's FVC is about 2.0 L to about 5.3 L (e.g., about 2.4 L to about 5.1 L; 3.3 L; or 3.6 L). In still another embodiment, prior to administering the pharmaceutical formulation, the subject's FVC measurement is either a pre-bronchodilator measurement or a post-bronchodilator measurement.

In an embodiment, prior to administering the pharmaceutical formulation, the subject's FVC is less than or equal to about 130% of predicted normal (e.g., about 70% to about 111%; about 80% to about 127%; about 91.2%; and about 98.8%). In another embodiment, prior to administering the pharmaceutical formulation, the subject's FVC is less than or equal to about 120% of predicted normal (e.g., about 70% to about 111%; about 91.2%; and about 98.8%). In yet another embodiment, prior to administering the pharmaceutical formulation, the subject's FVC is less than or equal to about 115% of predicted normal (e.g., about 70% to about 111%; about 91.2%; and about 98.8%). In still another embodiment, prior to administering the pharmaceutical formulation, the subject's FVC is less than or equal to about 110% of predicted normal (e.g., about 70% to about 111%; about 91.2%; and about 98.8%). In an embodiment, prior to administering the pharmaceutical formulation, the subject's FVC is less than or equal to about 100% of predicted normal (e.g., about 91.2% and about 98.8%). In another embodiment, prior to administering the pharmaceutical formulation, the subject's FVC is less than or equal to about 95% of predicted normal (e.g., about 91.2% and about 98.8%). In still another embodiment, prior to administering the pharmaceutical formulation, the subject's FVC measurement is either a pre-bronchodilator measurement or a post-bronchodilator measurement.

In an embodiment, prior to administering the pharmaceutical formulation, the subject's average FVC is about 90.0% to about 100.0% of predicted normal (e.g., 91.2% or 98.8%). In another embodiment, prior to administering the pharmaceutical formulation, the subject's average FVC is about 91.0% to about 99.0% of predicted normal (e.g., 91.2% to 98.8%). In yet another embodiment, prior to administering the pharmaceutical formulation, the subject's average FVC is about 91.2%. In still another embodiment, prior to administering the pharmaceutical formulation, the subject's average FVC is about 98.8%. In yet another embodiment, prior to administering the pharmaceutical formulation, the subject's FVC is about 65% to about 115% of predicted normal. In an embodiment, prior to administering the pharmaceutical formulation, the subject's FVC is about 65% to about 130% of predicted normal. In still another embodiment, prior to administering the pharmaceutical formulation, the subject's FVC is about 75% to about 130% of predicted normal. In an embodiment, prior to administering the pharmaceutical formulation, the subject's FVC measurement is either a pre-bronchodilator measurement or a post-bronchodilator measurement.

In an embodiment, prior to administering the pharmaceutical formulation, the subject's ratio of FEV₁ to forced vital capacity (FEV₁/FVC) is less than or equal to about 0.70 (e.g., about 0.46 to about 0.66%; about 0.378 to about 0.593; about 0.46 to about 0.66; about 0.507; and about 0.548). In another embodiment, prior to administering the pharmaceutical formulation, the subject's FEV₁/FVC is less than or equal to about 0.65 (e.g., about 0.378 to about 0.593; about 0.507; and about 0.548). In yet another embodiment, prior to administering the pharmaceutical formulation, the subject's FEV₁/FVC is less than or equal to about 0.60 (e.g., about 0.378 to about 0.593; about 0.507; and about 0.548). In still another embodiment, prior to administering the pharmaceutical formulation, the subject's FEV₁/FVC is less than or equal to about 0.50.

In an embodiment, prior to administering the pharmaceutical formulation, the subject's ratio of post-bronchodilator FEV₁ to forced vital capacity (FEV₁/FVC) is less than or equal to about 0.70. In another embodiment, prior to administering the pharmaceutical formulation, the subject's post-bronchodilator FEV₁/FVC is less than or equal to about 0.65. In yet another embodiment, prior to administering the pharmaceutical formulation, the subject's post-bronchodilator FEV₁/FVC is less than or equal to about 0.60. In still another embodiment, prior to administering the pharmaceutical formulation, the subject's post-bronchodilator FEV₁/FVC is less than or equal to about 0.50.

In an embodiment, prior to administering the pharmaceutical formulation, the subject's FEV₁/FVC is less than or equal to about 0.45. In another embodiment, prior to administering the pharmaceutical formulation, the subject's FEV₁/FVC is less than or equal to about 40%. In yet another embodiment, prior to administering the pharmaceutical formulation, the subject's average FEV₁/FVC is about 0.55 (e.g., 0.548). In an embodiment, prior to administering the pharmaceutical formulation, the subject's average FEV₁/FVC is about 0.50 to about 0.55 (e.g., 0.507 to 0.548). In an embodiment, prior to administering the pharmaceutical formulation, the subject's FEV₁/FVC is about 0.45 to about 0.68. In another embodiment, prior to administering the pharmaceutical formulation, the subject's FEV₁/FVC is about 0.35 to about 0.70. In another embodiment, prior to administering the pharmaceutical formulation, the subject's FEV₁/FVC is about 0.35 to about 0.60. In yet another embodiment, prior to administering the pharmaceutical formulation, the subject's FEV₁/FVC measurement is either a pre-bronchodilator measurement or a post-bronchodilator measurement.

In an embodiment, prior to administering the pharmaceutical formulation, the subject's post-bronchodilator FEV₁/FVC is less than or equal to about 0.45. In another embodiment, prior to administering the pharmaceutical formulation, the subject's post-bronchodilator FEV₁/FVC is less than or equal to about 0.40. In yet another embodiment, prior to administering the pharmaceutical formulation, the subject's average post-bronchodilator FEV₁/FVC is about 0.55. In an embodiment, prior to administering the pharmaceutical formulation, the subject's post-bronchodilator FEV₁/FVC is about 0.45 to about 0.68.

In another embodiment, prior to administering the pharmaceutical formulation, the subject's forced expiratory flow between 25% and 75% of forced vital capacity (FEF₂₅₋₇₅) is less than or equal to about 1.5 L/sec (e.g., about 0.3 L/sec to about 1.2 L/sec and about 0.6 L/sec). In yet another embodiment, prior to administering the pharmaceutical formulation, the subject's FEF₂₅₋₇₅ is less than or equal to about 1.2 L/sec (e.g., 0.3 L/sec to 1.2 L/sec and about 0.6 L/sec). In still another embodiment, prior to administering the pharmaceutical formulation, the subject's FEF₂₅₋₇₅ is less than or equal to about 0.6 L/sec. In still another embodiment, prior to administering the pharmaceutical formulation, the subject's average FEF₂₅₋₇₅ is about 0.6 L/sec. In an embodiment, prior to administering the pharmaceutical formulation, the subject's FEF₂₅₋₇₅ is about 0.2 L/sec to about 1.3 L/sec. In an embodiment, prior to administering the pharmaceutical formulation, the subject's FEF₂₅₋₇₅ measurement is either a pre-bronchodilator measurement or a post-bronchodilator measurement.

In another embodiment, after administering the pharmaceutical formulation, the subject's percent change in FVC is greater than or equal to about +0.15%. In yet another embodiment, after administering the pharmaceutical formulation, the subject's percent change in FVC is greater than or equal to about +0.2% (e.g., about +0.2% to about +6.6%; +0.2%; about +5.5%; about +2.5%; about +4.1%; about +5.5%; and about +6.6%). In still another embodiment, after administering the pharmaceutical formulation, the subject's percent change in FVC is greater than or equal to about +2.5% (e.g., about +2.5% to about +6.6%; about +2.5%; about +4.1%; about +5.5%; and about +6.6%). In another embodiment, after administering the pharmaceutical formulation, the subject's percent change in FVC is greater than or equal to about +4.1% (e.g., about +4.1% to about +6.6%; +4.1%; about +5.5%; and about +6.6%). In an embodiment, after administering the pharmaceutical formulation, the subject's percent change in FVC is greater than or equal to about +5.5% (e.g., about +5.5% to about +6.6%; +5.5%; and about +6.6%). In another embodiment, after administering the pharmaceutical formulation, the subject's percent change in FVC is greater than or equal to about +6.6%. In yet another embodiment, the subject's percent change in FVC is about 0.15% to about +19.0%.

In still another embodiment, the subject's maximum percent change in FVC is greater than or equal to about +9.8% (e.g., about 9.8% to about 18.8%). In an embodiment, the subject's maximum percent change in FVC is greater than or equal to about +13.5% (e.g., about 13.5% to about 18.8%). In another embodiment, the subject's maximum percent change in FVC is greater than or equal to about +14.0% (e.g., about 14.0% to about 18.8%). In yet another embodiment, the subject's maximum percent change in FVC is greater than or equal to about +17.8% (e.g., about 17.8% to about 18.8%). In still another embodiment, the subject's maximum percent change in FVC is greater than or equal to about +18.3% (e.g., about 18.3% to about 18.8%). In an embodiment, the subject's maximum percent change in FVC is greater than or equal to about +18.8%. In another embodiment, the subject's maximum percent change in FVC is about +18.8%.

In still another embodiment, after administering the pharmaceutical formulation, the subject's change in FVC is greater than or equal to about +50 mL (e.g., about 50 mL to about +215 mL). In an embodiment, after administering the pharmaceutical formulation, the subject's change in FVC is greater than or equal to about +70 mL (e.g., about 70 mL to about +215 mL). In another embodiment, after administering the pharmaceutical formulation, the subject's change in FVC is greater than or equal to about +72 mL (e.g., about 72 mL to about +215 mL). In yet another embodiment, after administering the pharmaceutical formulation, the subject's change in FVC is greater than or equal to about +92 mL (e.g., about 92 mL to about +215 mL). In still another embodiment, after administering the pharmaceutical formulation, the subject's change in FVC is greater than or equal to about +162 mL (e.g., about 162 mL to about +215 mL). In an embodiment, after administering the pharmaceutical formulation, the subject's change in FVC is greater than or equal to about +210 mL (e.g., about 210 mL to about +215 mL). In another embodiment, after administering the pharmaceutical formulation, the subject's change in FVC is about +70 mL to about +215 mL.

In yet another embodiment, after administering the pharmaceutical formulation, the subject's maximum change in FVC is greater than or equal to about +375 mL (e.g., about +375 mL to about +1135 mL). In still another embodiment, after administering the pharmaceutical formulation, the subject's maximum change in FVC is greater than or equal to about +408 mL (e.g., about +408 mL to about +1135 mL). In an embodiment, after administering the pharmaceutical formulation, the subject's maximum change in FVC is greater than or equal to about +423 mL (e.g., about +423 mL to about +1135 mL). In another embodiment, after administering the pharmaceutical formulation, the subject's maximum change in FVC is greater than or equal to about +565 mL (e.g., about +565 mL to about +1135 mL). In yet another embodiment, after administering the pharmaceutical formulation, the subject's maximum change in FVC is greater than or equal to about +630 mL (e.g., about +630 mL to about +1135 mL). In still another embodiment, after administering the pharmaceutical formulation, the subject's maximum change in FVC is greater than or equal to about +1130 mL (e.g., about +1130 mL to about +1135 mL). In an embodiment, after administering the pharmaceutical formulation, the subject's maximum change in FVC is about +1130 mL. In another embodiment, after administering the pharmaceutical formulation, the subject's maximum change in FVC is about +370 mL to about +1135 mL.

In an embodiment, after administering the pharmaceutical formulation, the subject's percent change in FEV₁ is greater than or equal to about +5% (e.g., about +5% to about 10.5% and about +5% to about 25.0%). In another embodiment, after administering the pharmaceutical formulation, the subject's percent change in FEV₁ is greater than or equal to about +7% (e.g., about +7% to about 10.5% and about +7% to about 25.0%). In yet another embodiment, after administering the pharmaceutical formulation, the subject's percent change in FEV₁ is greater than or equal to about +10% (e.g., about +10% to about 10.5% and about +10% to about 25.0%). In still another embodiment, after administering the pharmaceutical formulation, the subject's percent change in FEV₁ is greater than or equal to about +15% (e.g., about +15% to about 25.0%). In an embodiment, after administering the pharmaceutical formulation, the subject's percent change in FEV₁ is greater than or equal to about +20% (e.g., about +20% to about 25.0%).

In another embodiment, after administering the pharmaceutical formulation, the subject's percent change in FEV₁ is greater than or equal to about +25%. In yet another embodiment, after administering the pharmaceutical formulation, the subject's percent change in FEV₁ is about 5% to about 12%. In still another embodiment, after administering the pharmaceutical formulation, the subject's percent change in FEV₁ is about 5% to about 28%. In another embodiment, the subject's maximum change in FEV₁ is about 15% to about 25%.

In an embodiment, after administering the pharmaceutical formulation, the subject's placebo-corrected change in FEV₁ is greater than or equal to about +40 mL. In another embodiment, after administering the pharmaceutical formulation, the subject's placebo-corrected change in FEV₁ is greater than or equal to about +50 mL.

In yet another embodiment, after administering the pharmaceutical formulation, the subject's change in FEV₁ is greater than or equal to about +70 mL (e.g., about +80 mL to about +300 mL and about +110 mL to about +420 mL). In an embodiment, after administering the pharmaceutical formulation, the subject's change in FEV₁ is greater than or equal to about +80 mL (e.g., about +80 mL to about +300 mL and about +110 mL to about +420 mL). In still another embodiment, after administering the pharmaceutical formulation, the subject's change in FEV₁ is greater than or equal to about +100 mL (e.g., about +100 mL to about +300 mL and about +110 mL to about +420 mL). In an embodiment, after administering the pharmaceutical formulation, the subject's change in FEV₁ is greater than or equal to about +120 mL (e.g., about +120 mL to about +300 mL and about +120 mL to about +420 mL). In another embodiment, after administering the pharmaceutical formulation, the subject's change in FEV₁ is greater than or equal to about +140 mL (e.g., about +140 mL to about +300 mL and about +140 mL to about +420 mL).

In still another embodiment, after administering the pharmaceutical formulation, the subject's placebo-corrected change in FEV₁ is greater than or equal to about +100 mL. In an embodiment, after administering the pharmaceutical formulation, the subject's placebo-corrected change in FEV₁ is greater than or equal to about +120 mL. In another embodiment, after administering the pharmaceutical formulation, the subject's placebo-corrected change in FEV₁ is greater than or equal to about +140 mL.

In yet another embodiment, after administering the pharmaceutical formulation, the subject's change in FEV₁ is greater than or equal to about +160 mL (e.g., about +160 mL to about +300 mL and about +160 mL to about +420 mL). In still another embodiment, after administering the pharmaceutical formulation, the subject's change in FEV₁ is greater than or equal to about +180 mL (e.g., about +180 mL to about +300 mL and about +180 mL to about +420 mL). In an embodiment, wherein, after administering the pharmaceutical formulation, the subject's change in FEV₁ is greater than or equal to about +190 mL (e.g., about +190 mL to about +300 mL and about +190 mL to about +420 mL).

In yet another embodiment, after administering the pharmaceutical formulation, the subject's placebo-corrected change in FEV₁ is greater than or equal to about +160 mL. In still another embodiment, after administering the pharmaceutical formulation, the subject's placebo-corrected change in FEV₁ is greater than or equal to about +180 mL. In an embodiment, wherein, after administering the pharmaceutical formulation, the subject's placebo-corrected change in FEV₁ is greater than or equal to about +190 mL.

In another embodiment, after administering the pharmaceutical formulation, the subject's change in FEV₁ is greater than or equal to about +200 mL (e.g., about +200 mL to about +300 mL and about +200 mL to about +420 mL). In yet another embodiment, after administering the pharmaceutical formulation, the subject's change in FEV₁ is greater than or equal to about +210 mL (e.g., about +210 mL to about +300 mL and about +210 mL to about +420 mL). In still another embodiment, after administering the pharmaceutical formulation, the subject's change in FEV₁ is greater than or equal to about +220 mL (e.g., about +220 mL to about +300 mL and about +220 mL to about +420 mL).

In another embodiment, after administering the pharmaceutical formulation, the subject's placebo-corrected change in FEV₁ is greater than or equal to about +200 mL. In yet another embodiment, after administering the pharmaceutical formulation, the subject's placebo-corrected change in FEV₁ is greater than or equal to about +210 mL. In still another embodiment, after administering the pharmaceutical formulation, the subject's placebo-corrected change in FEV₁ is greater than or equal to about +220 mL.

In an embodiment, after administering the pharmaceutical formulation, the subject's change in FEV₁ is greater than or equal to about +240 mL (e.g., about +240 mL to about +300 mL and about +240 mL to about +420 mL). In another embodiment, after administering the pharmaceutical formulation, the subject's change in FEV₁ is greater than or equal to about +300 mL (e.g., about +300 mL to about +420 mL). In yet another embodiment, after administering the pharmaceutical formulation, the subject's change in FEV₁ is greater than or equal to about +390 mL (e.g., about +390 mL to about +420 mL). In still another embodiment, after administering the pharmaceutical formulation, the subject's change in FEV₁ is greater than or equal to about +400 mL (e.g., +400 mL).

In an embodiment, after administering the pharmaceutical formulation, the subject's placebo-corrected change in FEV₁ is greater than or equal to about +240 mL. In another embodiment, after administering the pharmaceutical formulation, the subject's placebo-corrected change in FEV₁ is greater than or equal to about +300 mL. In yet another embodiment, after administering the pharmaceutical formulation, the subject's placebo-corrected change in FEV₁ is greater than or equal to about +390 mL. In still another embodiment, after administering the pharmaceutical formulation, the subject's placebo-corrected change in FEV₁ is greater than or equal to about +400 mL.

In an embodiment, after administering the pharmaceutical formulation, the subject's change in FEV₁ is greater than or equal to about +410 mL (e.g., about +410 mL to about +650 mL). In another embodiment, after administering the pharmaceutical formulation, the subject's change in FEV₁ is greater than or equal to about +420 mL (e.g., about +420 mL to about +650 mL). In yet another embodiment, after administering the pharmaceutical formulation, the subject's change in FEV₁ is greater than or equal to about +500 mL (e.g., about +500 mL to about +650 mL).

In an embodiment, after administering the pharmaceutical formulation, the subject's placebo-corrected change in FEV₁ is greater than or equal to about +410 mL. In another embodiment, after administering the pharmaceutical formulation, the subject's placebo-corrected change in FEV₁ is greater than or equal to about +420 mL. In yet another embodiment, after administering the pharmaceutical formulation, the subject's placebo-corrected change in FEV₁ is greater than or equal to about +500 mL.

In still another embodiment, after administering the pharmaceutical formulation, the subject's change in FEV₁ is greater than or equal to about +550 mL (e.g., about +550 mL to about +650 mL). In an embodiment, after administering the pharmaceutical formulation, the subject's change in FEV₁ is greater than or equal to about +570 mL (e.g., about +570 mL to about +650 mL). In another embodiment, after administering the pharmaceutical formulation, the subject's change in FEV₁ is greater than or equal to about +600 mL (e.g., about +600 mL to about +650 mL).

In still another embodiment, after administering the pharmaceutical formulation, the subject's placebo-corrected change in FEV₁ is greater than or equal to about +550 mL. In an embodiment, after administering the pharmaceutical formulation, the subject's placebo-corrected change in FEV₁ is greater than or equal to about +570 mL. In another embodiment, after administering the pharmaceutical formulation, the subject's placebo-corrected change in FEV₁ is greater than or equal to about +600 mL.

In yet another embodiment, after administering the pharmaceutical formulation, the subject's change in FEV₁ is greater than or equal to about +650 mL (e.g., +650 mL). In an embodiment, after administering the pharmaceutical formulation, the subject's average change in FEV₁ is about +70 mL to about +310 mL. In still another embodiment, after administering the pharmaceutical formulation, the subject's average change in FEV₁ is about +120 mL to about +260 mL. In an embodiment, after administering the pharmaceutical formulation, the subject's average change in FEV₁ is about +140 mL to about +240 mL. In an embodiment, after administering the pharmaceutical formulation, the subject's change in FEV₁ is about +100 mL to about +430 mL. In another embodiment, after administering the pharmaceutical formulation, the subject's average change in FEV₁ is about 250 mL. In yet another embodiment, after administering the pharmaceutical formulation, the subject's average change in FEV₁ is about 130 mL.

In yet another embodiment, after administering the pharmaceutical formulation, the subject's placebo-corrected change in FEV₁ is greater than or equal to about +650 mL. In still another embodiment, after administering the pharmaceutical formulation, the subject's average placebo-corrected change in FEV₁ is about +120 mL to about +260 mL. In an embodiment, after administering the pharmaceutical formulation, the subject's average placebo-corrected change in FEV₁ is about +140 mL to about +240 mL. In an embodiment, after administering the pharmaceutical formulation, the subject's placebo-corrected change in FEV₁ is about +100 mL to about +430 mL. In another embodiment, after administering the pharmaceutical formulation, the subject's average placebo-corrected change in FEV₁ is about 250 mL.

In yet another embodiment, after administering the pharmaceutical formulation, the subject's percent change in FEV₁/FVC is greater than or equal to about +2.00% (e.g., about +2.00% to about +7.00%). In an embodiment, after administering the pharmaceutical formulation, the subject's percent change in FEV₁/FVC is greater than or equal to about +2.3% (e.g., about +2.3% to about +7.0%). In still another embodiment, after administering the pharmaceutical formulation, the subject's percent change in FEV₁/FVC is greater than or equal to about +2.34% (e.g., about +2.34% to about +7.00%). In another embodiment, after administering the pharmaceutical formulation, the subject's percent change in FEV₁/FVC is greater than or equal to about +3.5% (e.g., about +2.3% to about +7.0%). In yet another embodiment, after administering the pharmaceutical formulation, the subject's percent change in FEV₁/FVC is greater than or equal to about +3.8% (e.g., about +2.3% to about +7.0%). In an embodiment, after administering the pharmaceutical formulation, the subject's percent change in FEV₁/FVC is greater than or equal to about +3.81% (e.g., about +3.81% to about +7.00%). In another embodiment, after administering the pharmaceutical formulation, the subject's percent change in FEV₁/FVC is greater than or equal to about +3.83% (e.g., about +3.83% to about +7.00%).

In still another embodiment, after administering the pharmaceutical formulation, the subject's percent change in FEV₁/FVC is greater than or equal to about +3.91% (e.g., about +3.91% to about +7.00%). In another embodiment, after administering the pharmaceutical formulation, the subject's percent change in FEV₁/FVC is greater than or equal to about +4.0% (e.g., about +4.0% to about +7.0%). In an embodiment, after administering the pharmaceutical formulation, the subject's percent change in FEV₁/FVC is greater than or equal to about +4.01% (e.g., about +4.01% to about +7.00%). In another embodiment, after administering the pharmaceutical formulation, the subject's percent change in FEV₁/FVC is greater than or equal to about +5.98% (e.g., +5.98%). In yet another embodiment, after administering the pharmaceutical formulation, the subject's percent change in FEV₁/FVC is about +2.20% to about 6.20%.

In still another embodiment, after administering the pharmaceutical formulation, the subject's maximum percent change in FEV₁/FVC is greater than or equal to about +6.00% (e.g., about +6.00% to about +9.50%). In an embodiment, after administering the pharmaceutical formulation, the subject's maximum percent change in FEV₁/FVC is greater than or equal to about +6.05% (e.g., about +6.05% to about +9.50%). In another embodiment, after administering the pharmaceutical formulation, the subject's maximum percent change in FEV₁/FVC is greater than or equal to about +6.85% (e.g., about +6.85% to about +9.50%). In yet another embodiment, after administering the pharmaceutical formulation, the subject's maximum percent change in FEV₁/FVC is greater than or equal to about +7.35% (e.g., about +7.35% to about +9.50%).

In yet another embodiment, after administering the pharmaceutical formulation, the subject's maximum percent change in FEV₁/FVC is greater than or equal to about +7.38% (e.g., about +7.38% to about +9.50%). In still another embodiment, after administering the pharmaceutical formulation, the subject's maximum percent change in FEV₁/FVC is greater than or equal to about +8.15% (e.g., about +8.15% to about +9.50%). In an embodiment, after administering the pharmaceutical formulation, the subject's maximum percent change in FEV₁/FVC is greater than or equal to about +8.78% (e.g., about +8.78% to about +9.50%).

In another embodiment, after administering the pharmaceutical formulation, the subject's maximum percent change in FEV₁/FVC is about +8.78%. In yet another embodiment, after administering the pharmaceutical formulation, the subject's maximum percent change in FEV₁/FVC is about +5.90% to about +8.90%.

In still another embodiment, after administering the pharmaceutical formulation, the subject's change in FEF₂₅₋₇₅ is greater than or equal to about +0.01 L/sec (e.g., about +0.01 L/sec to about +0.30 L/sec and about +0.09 L/sec to about +0.40). In an embodiment, after administering the pharmaceutical formulation, the subject's change in FEF₂₅₋₇₅ is greater than or equal to about +0.03 L/sec (e.g., about +0.03 L/sec to about +0.30 L/sec and about +0.09 L/sec to about +0.40). In still another embodiment, after administering the pharmaceutical formulation, the subject's change in FEF₂₅₋₇₅ is greater than or equal to about +0.04 L/sec (e.g., about +0.04 L/sec to about +0.30 L/sec and about +0.09 L/sec to about +0.40). In another embodiment, after administering the pharmaceutical formulation, the subject's change in FEF₂₅₋₇₅ is greater than or equal to about +0.05 L/sec (e.g., about +0.05 L/sec to about +0.30 L/sec and about +0.09 L/sec to about +0.40). In yet another embodiment, after administering the pharmaceutical formulation, the subject's change in FEF₂₅₋₇₅ is about +0.05 L/sec. In still another embodiment, after administering the pharmaceutical formulation, the subject's change in FEF₂₅₋₇₅ is about +0.02 L/sec to about 0.06 L/sec.

In yet another embodiment, after administering the pharmaceutical formulation, the subject's change in FEF₂₅₋₇₅ is greater than or equal to about +0.14 L/sec (e.g., 0.14 L/sec). In still another embodiment, after administering the pharmaceutical formulation, the subject's change in FEF₂₅₋₇₅ is greater than or equal to about +0.15 L/sec (e.g., 0.15 L/sec). In yet another embodiment, after administering the pharmaceutical formulation, the subject's change in FEF₂₅₋₇₅ is about +0.15 L/sec. In still another embodiment, after administering the pharmaceutical formulation, the subject's change in FEF₂₅₋₇₅ is about +0.12 L/sec to about 0.16 L/sec. In an embodiment, the subject has a blood eosinophil count that is greater than or equal to 300 cells/μL.

In still another embodiment, after administering the pharmaceutical formulation, the subject's change in FEF₂₅₋₇₅ is greater than or equal to about +0.01 L/sec (e.g., about +0.01 L/sec to about 0.35 L/sec). In an embodiment, after administering the pharmaceutical formulation, the subject's change in FEF₂₅₋₇₅ is greater than or equal to about +0.03 L/sec (e.g., about +0.03 L/sec to about 0.35 L/sec). In still another embodiment, after administering the pharmaceutical formulation, the subject's change in FEF₂₅₋₇₅ is greater than or equal to about +0.04 L/sec (e.g., about +0.04 L/sec to about 0.35 L/sec). In another embodiment, after administering the pharmaceutical formulation, the subject's change in FEF₂₅₋₇₅ is greater than or equal to about +0.08 L/sec (e.g., about +0.08 L/sec to about 0.35 L/sec). In an embodiment, after administering the pharmaceutical formulation, the subject's change in FEF₂₅₋₇₅ is about +0.11 L/sec (e.g., about +0.11 L/sec to about 0.35 L/sec). In another embodiment, after administering the pharmaceutical formulation, the subject's change in FEF₂₅₋₇₅ is about +0.30 L/sec (e.g., about +0.30 L/sec to about 0.35 L/sec). In yet another embodiment, after administering the pharmaceutical formulation, the subject's change in FEF₂₅₋₇₅ is about +0.08 L/sec. In still another embodiment, after administering the pharmaceutical formulation, the subject's change in FEF₂₅₋₇₅ is about +0.01 L/sec to about 0.35 L/sec.

In yet another embodiment, after administering the pharmaceutical formulation, the subject's change in FEF₂₅₋₇₅ is greater than or equal to about +0.09 L/sec (e.g., about +0.09 to about +0.42 L/sec). In still another embodiment, after administering the pharmaceutical formulation, the subject's change in FEF₂₅₋₇₅ is greater than or equal to about +0.11 L/sec (e.g., about +0.11 to about +0.42 L/sec). In yet another embodiment, after administering the pharmaceutical formulation, the subject's change in FEF₂₅₋₇₅ is greater than or equal to about +0.13 L/sec (e.g., about +0.13 to about +0.42 L/sec). In still another embodiment, after administering the pharmaceutical formulation, the subject's change in FEF₂₅₋₇₅ is greater than or equal to about +0.14 L/sec (e.g., about +0.14 to about +0.42 L/sec). In an embodiment, after administering the pharmaceutical formulation, the subject's change in FEF₂₅₋₇₅ is greater than or equal to about +0.21 L/sec (e.g., about +0.21 to about +0.42 L/sec). In another embodiment, after administering the pharmaceutical formulation, the subject's change in FEF₂₅₋₇₅ is greater than or equal to about +0.40 L/sec (e.g., about +0.40 to about +0.42 L/sec). In yet another embodiment, after administering the pharmaceutical formulation, the subject's change in FEF₂₅₋₇₅ is about +0.18 L/sec. In still another embodiment, after administering the pharmaceutical formulation, the subject's change in FEF₂₅₋₇₅ is about +0.07 to about +0.42 L/sec. In an embodiment, the subject has a blood eosinophil count that is less than 300 cells/μL.

In still another embodiment, after administering the pharmaceutical formulation, the subject's change in FEF₂₅₋₇₅ is greater than or equal to about +0.14 L/sec (e.g., about +0.14 L/sec to about +0.25 L/sec). In yet another embodiment, after administering the pharmaceutical formulation, the subject's change in FEF₂₅₋₇₅ is greater than or equal to about +0.15 L/sec (e.g., about +0.15 L/sec to about +0.25 L/sec). In an embodiment, after administering the pharmaceutical formulation, the subject's change in FEF₂₅₋₇₅ is greater than or equal to about +0.17 L/sec (e.g., about +0.17 L/sec to about +0.25 L/sec). In yet another embodiment, after administering the pharmaceutical formulation, the subject's change in FEF₂₅₋₇₅ is greater than or equal to about +0.20 L/sec (e.g., about +0.20 L/sec to about +0.25 L/sec). In another embodiment, after administering the pharmaceutical formulation, the subject's change in FEF₂₅₋₇₅ is greater than or equal to about +0.22 L/sec (e.g., about +0.22 L/sec to about +0.25 L/sec). In yet another embodiment, after administering the pharmaceutical formulation, the subject's change in FEF₂₅₋₇₅ is about +0.22 L/sec. In still another embodiment, after administering the pharmaceutical formulation, the subject's change in FEF₂₅₋₇₅ is about +0.12 L/sec to about +0.25 L/sec.

In an embodiment, after administering the pharmaceutical formulation, the subject's maximum change in FEF₂₅₋₇₅ is greater than or equal to about 0.20 L/sec (e.g., about 0.30 L/sec to about 0.62 L/sec). In another embodiment, after administering the pharmaceutical formulation, the subject's maximum change in FEF₂₅₋₇₅ is greater than or equal to about 0.30 L/sec (e.g., about 0.30 L/sec to about 0.62 L/sec). In yet another embodiment, after administering the pharmaceutical formulation, the subject's maximum change in FEF₂₅₋₇₅ is greater than or equal to about 0.32 L/sec (e.g., about 0.32 L/sec to about 0.62 L/sec). In still another embodiment, after administering the pharmaceutical formulation, the subject's maximum change in FEF₂₅₋₇₅ is greater than or equal to about 0.33 L/sec (e.g., about 0.33 L/sec to about 0.62 L/sec). In an embodiment, after administering the pharmaceutical formulation, the subject's maximum change in FEF₂₅₋₇₅ is greater than or equal to about 0.35 L/sec (e.g., about 0.35 L/sec to about 0.62 L/sec). In another embodiment, after administering the pharmaceutical formulation, the subject's maximum change in FEF₂₅₋₇₅ is greater than or equal to about 0.40 L/sec (e.g., about 0.40 L/sec to about 0.62 L/sec). In still another embodiment, after administering the pharmaceutical formulation, the subject's maximum change in FEF₂₅₋₇₅ is greater than or equal to about 0.44 L/sec (e.g., about 0.44 L/sec to about 0.62 L/sec). In an embodiment, after administering the pharmaceutical formulation, the subject's maximum change in FEF₂₅₋₇₅ is greater than or equal to about 0.60 L/sec (e.g., about 0.60 L/sec to about 0.62 L/sec). In another embodiment, after administering the pharmaceutical formulation, the subject's maximum change in FEF₂₅₋₇₅ is about 0.30 L/sec to about 0.62 L/sec. In yet another embodiment, the subject has a blood eosinophil count that is less than 300 cells/μL.

A number of cytokines and chemokines, including interleukin 5 (IL-5), control the maturation, trafficking, and activity of eosinophils (Crit. Rev. Immunol. 2016, 36, 429-444). Eosinophils secrete various proteins that promote inflammation and tissue remodeling. Studies have reported that COPD patients have increased numbers of eosinophils in sputum samples, broncho-alveolar lavage, and bronchial biopsies compared to healthy controls (Eur. Respir. J. 2000, 15, 109-115 and Int. J. Chron. Obstruct. Pulmon. Dis. 2006, 1, 39-47). Close inspection of the data shows that a subset of COPD patients have increased eosinophil numbers, while the remainder have levels similar to controls. Blood eosinophil numbers in COPD patients are also higher than age-matched healthy controls, even when patients with a history of asthma or atopy are excluded (Eur. Respir. J. 2019, 54, 1900633).

Further, COPD patients with higher blood and lung eosinophil counts showed numerous other pathological differences, including increased levels of biomarkers of type 2 (T2) inflammation and greater reticular basement membrane thickening. These features are also seen in patients with asthma (Nat. Rev. Immunol. 2015, 15, 57-65 and Allergol. Int. 2008, 57, 11-19). Furthermore, a study comparing COPD patients with a confirmed childhood history of asthma versus COPD patients with increased eosinophils and no history of asthma showed that the former group had more evidence of allergy and more exacerbations while displaying less eosinophilic inflammation (Respir. Res. 2017, 18, 73). These data highlight that the terms “asthma” and “eosinophilic” should not be used interchangeably in COPD patients.

In still another embodiment, prior to administering the pharmaceutical formulation, the subject's blood eosinophil count as expressed in units of cells/μL is greater than or equal to about 70. In an embodiment, prior to administering the pharmaceutical formulation, the subject's blood eosinophil count as expressed in units of cells/μL is greater than or equal to about 100. In another embodiment, prior to administering the pharmaceutical formulation, the subject's blood eosinophil count as expressed in units of cells/μL is greater than or equal to about 200. In yet another embodiment, prior to administering the pharmaceutical formulation, the subject's blood eosinophil count as expressed in units of cells/μL is greater than or equal to about 250.

In still another embodiment, prior to administering the pharmaceutical formulation, the subject's blood eosinophil count as expressed in units of cells/μL is greater than or equal to about 300. In an embodiment, prior to administering the pharmaceutical formulation, the subject's blood eosinophil count as expressed in units of cells/μL is greater than or equal to about 400. In another embodiment, prior to administering the pharmaceutical formulation, the subject's blood eosinophil count as expressed in units of cells/μL is greater than or equal to about 450.

In yet another embodiment, prior to administering the pharmaceutical formulation, the subject's blood eosinophil count as expressed in units of cells/μL is greater than or equal to about 500. In an embodiment, prior to administering the pharmaceutical formulation, the subject's blood eosinophil count as expressed in units of cells/μL is less than about 70. In still another embodiment, prior to administering the pharmaceutical formulation, the subject's average blood eosinophil count as expressed in units of cells/μL is about 235. In an embodiment, prior to administering the pharmaceutical formulation, the subject's average blood eosinophil count as expressed in units of cells/μL is about 405. In an embodiment, prior to administering the pharmaceutical formulation, the subject's average blood eosinophil count as expressed in units of cells/μL is about 320 to about 490.

In another embodiment, prior to administering the pharmaceutical formulation, the subject's blood eosinophil count as expressed in units of cells/μL is less than about 300 (e.g., about 60 to about 230). In yet another embodiment, prior to administering the pharmaceutical formulation, the subject's blood eosinophil count as expressed in units of cells/μL is less than 300 (e.g., about 60 to about 230). In still another embodiment, prior to administering the pharmaceutical formulation, the subject's blood eosinophil count as expressed in units of cells/μL is less than or equal to about 250 (e.g., about 60 to about 230).

In an embodiment, prior to administering the pharmaceutical formulation, the subject's blood eosinophil count as expressed in units of cells/μL is less than or equal to about 200 (e.g., about 60 to about 230). In another embodiment, prior to administering the pharmaceutical formulation, the subject's blood eosinophil count as expressed in units of cells/μL is less than or equal to about 100 (e.g., about 60 to about 100). In yet another embodiment, prior to administering the pharmaceutical formulation, the subject's blood eosinophil count as expressed in units of cells/μL is less than or equal to about 70 (e.g., about 60 to about 70). In still another embodiment, prior to administering the pharmaceutical formulation, the subject's average blood eosinophil count as expressed in units of cells/μL is about 150. In an embodiment, prior to administering the pharmaceutical formulation, the subject's average blood eosinophil count as expressed in units of cells/μL is about 60 to about 230.

Fractional exhaled nitric oxide (FENO), a biomarker synthesized endogenously in the human respiratory tract, plays a key role in physiological regulation of airway function, including neurotransmission, vasodilation, and immunoregulation (Immunol. Allergy Clin. North Am. 2007, 27, 571-586). Moreover, FENO is implicated in the pathophysiology of airway disease, and elevated FENO level is found in inflammatory lung disorders such as asthma, pulmonary hypertension, and cystic fibrosis (J. Asthma 2016, 53, 404-412). The measurement of FENO has been suggested as a simple and noninvasive test in eosinophilic airway inflammation, and the utility in aiding diagnosis and monitoring of asthma has also been confirmed (J. Asthma 2016, 53, 404-412 and J. Asthma 2007, 44, 805-810). Recent studies suggest that FeNO may be a useful prognostic biomarker for predicting clinical outcomes in patients with COPD (Respir. Med. 2024, 229, 107682).

In an embodiment, prior to administering the pharmaceutical formulation, the subject's fractional exhaled nitric oxide (FeNO) concentration is greater than or equal to about 5.0 ppb. In another embodiment, prior to administering the pharmaceutical formulation, the subject's FeNO concentration is greater than or equal to about 15 ppb. In yet another embodiment, prior to administering the pharmaceutical formulation, the subject's FeNO concentration is greater than or equal to about 20 ppb. In still another embodiment, prior to administering the pharmaceutical formulation, the subject's FeNO concentration is greater than or equal to about 25 ppb.

In an embodiment, prior to administering the pharmaceutical formulation, the subject's FeNO concentration is greater than or equal to about 30 ppb. In another embodiment, prior to administering the pharmaceutical formulation, the subject's FeNO concentration is greater than or equal to about 45 ppb. In yet another embodiment, prior to administering the pharmaceutical formulation, the subject's FeNO concentration is greater than or equal to about 60 ppb. In still another embodiment, prior to administering the pharmaceutical formulation, the subject's FeNO concentration is greater than or equal to about 71 ppb.

In an embodiment, prior to administering the pharmaceutical formulation, the subject's FeNO concentration is greater than or equal to about 75 ppb. In another embodiment, prior to administering the pharmaceutical formulation, the subject's average FeNO concentration is about 30 ppb.

In yet another embodiment, after administering the pharmaceutical formulation, the subject's percent change in FeNO concentration is less than or equal to about −0.5%. In still another embodiment, after administering the pharmaceutical formulation, the subject's placebo-corrected percent change in FeNO concentration is less than or equal to about −5.0%.

In yet another embodiment, after administering the pharmaceutical formulation, the subject's percent change in FeNO concentration is less than or equal to about −10.0%. In still another embodiment, after administering the pharmaceutical formulation, the subject's percent change in FeNO concentration is less than or equal to about −11.0%. In an embodiment, after administering the pharmaceutical formulation, the subject's percent change in FeNO concentration is less than or equal to about −15.0%.

In yet another embodiment, after administering the pharmaceutical formulation, the subject's placebo-corrected percent change in FeNO concentration is less than or equal to about −10.0%. In still another embodiment, after administering the pharmaceutical formulation, the subject's placebo-corrected percent change in FeNO concentration is less than or equal to about −11.0%. In an embodiment, after administering the pharmaceutical formulation, the subject's placebo-corrected percent change in FeNO concentration is less than or equal to about −15.0%.

In yet another embodiment, after administering the pharmaceutical formulation, the subject's percent change in FeNO concentration is less than or equal to about −20.0%. In an embodiment, after administering the pharmaceutical formulation, the subject's percent change in FeNO concentration is less than or equal to about −25.0%. In still another embodiment, after administering the pharmaceutical formulation, the subject's percent change in FeNO concentration is less than or equal to about −30.0%.

In yet another embodiment, after administering the pharmaceutical formulation, the subject's placebo-corrected percent change in FeNO concentration is less than or equal to about −20.0%. In still another embodiment, after administering the pharmaceutical formulation, the subject's placebo-corrected percent change in FeNO concentration is less than or equal to about −30.0%.

In an embodiment, after administering the pharmaceutical formulation, the subject's percent change in FeNO concentration is less than or equal to about −35.0%. In another embodiment, after administering the pharmaceutical formulation, the subject's percent change in FeNO concentration is less than or equal to about −45.0%. In yet another embodiment, after administering the pharmaceutical formulation, the subject's percent change in FeNO concentration is less than or equal to about −50.0%.

In an embodiment, after administering the pharmaceutical formulation, the subject's placebo-corrected percent change in FeNO concentration is less than or equal to about −35.0%. In another embodiment, after administering the pharmaceutical formulation, the subject's placebo-corrected percent change in FeNO concentration is less than or equal to about −45.0%. In yet another embodiment, after administering the pharmaceutical formulation, the subject's placebo-corrected percent change in FeNO concentration is less than or equal to about −50.0%.

In still another embodiment, after administering the pharmaceutical formulation, the subject's percent change in FeNO concentration is less than or equal to about −55.0%. In an embodiment, after administering the pharmaceutical formulation, the subject's percent change in FeNO concentration is less than or equal to about −60.0%. In another embodiment, after administering the pharmaceutical formulation, the subject's percent change in FeNO concentration is less than or equal to about −70.0%.

In still another embodiment, after administering the pharmaceutical formulation, the subject's placebo-corrected percent change in FeNO concentration is less than or equal to about −55.0%. In an embodiment, after administering the pharmaceutical formulation, the subject's placebo-corrected percent change in FeNO concentration is less than or equal to about −60.0%. In another embodiment, after administering the pharmaceutical formulation, the subject's placebo-corrected percent change in FeNO concentration is less than or equal to about −70.0%.

In yet another embodiment, after administering the pharmaceutical formulation, the subject's percent change in FeNO concentration is less than or equal to about −75.0%. In still another embodiment, after administering the pharmaceutical formulation, the subject's percent change in FeNO concentration is less than or equal to about −80.0%.

In yet another embodiment, after administering the pharmaceutical formulation, the subject's placebo-corrected percent change in FeNO concentration is less than or equal to about −75.0%. In still another embodiment, after administering the pharmaceutical formulation, the subject's placebo-corrected percent change in FeNO concentration is less than or equal to about −80.0%.

In an embodiment, after administering the pharmaceutical formulation, the subject's percent change in FeNO concentration is less than or equal to about −85.0%. In another embodiment, after administering the pharmaceutical formulation, the subject's percent change in FeNO concentration is less than or equal to about −88.0%. In yet another embodiment, after administering the pharmaceutical formulation, the subject's average percent change in FeNO concentration is about −30.0% to about −50.0%. In still another embodiment, after administering the pharmaceutical formulation, the subject's average percent change in FeNO concentration is about −38.0% to about −42.5%.

In an embodiment, after administering the pharmaceutical formulation, the subject's placebo-corrected percent change in FeNO concentration is less than or equal to about −85.0%. In another embodiment, after administering the pharmaceutical formulation, the subject's placebo-corrected percent change in FeNO concentration is less than or equal to about −88.0%. In yet another embodiment, after administering the pharmaceutical formulation, the subject's average placebo-corrected percent change in FeNO concentration is about −30.0% to about −50.0%. In still another embodiment, after administering the pharmaceutical formulation, the subject's average placebo-corrected percent change in FeNO concentration is about −38.0% to about −42.5%.

In an embodiment, after administering the pharmaceutical formulation, the subject's percent change in FeNO concentration is about −5.5% to about −80.0%. In another embodiment, after administering the pharmaceutical formulation, the subject's percent change in FeNO concentration is about −5.5% to about −70.0%. In yet another embodiment, the subjects percent change in FeNO concentration is about −0.5% to about −28.0%. In still another embodiment, the subjects percent change in FeNO concentration is about −22.0% to about −35.0%.

In an embodiment, after administering the pharmaceutical formulation, the subject's placebo-corrected percent change in FeNO concentration is about −5.5% to about −80.0%. In another embodiment, after administering the pharmaceutical formulation, the subject's placebo-corrected percent change in FeNO concentration is about −5.5% to about −70.0%.

In an embodiment, after administering the pharmaceutical formulation, the subject's change in FeNO concentration is less than or equal to about −1.0 ppb. In another embodiment, after administering the pharmaceutical formulation, the subject's change in FeNO concentration is less than or equal to about −1.5 ppb. In yet another embodiment, after administering the pharmaceutical formulation, the subject's change in FeNO concentration is less than or equal to about −3.0 ppb. In still another embodiment, after administering the pharmaceutical formulation, the subject's change in FeNO concentration is less than or equal to about −10.0 ppb.

In an embodiment, after administering the pharmaceutical formulation, the subject's placebo-corrected change in FeNO concentration is less than or equal to about −1.0 ppb. In another embodiment, after administering the pharmaceutical formulation, the subject's placebo-corrected change in FeNO concentration is less than or equal to about −1.5 ppb. In yet another embodiment, after administering the pharmaceutical formulation, the subject's placebo-corrected change in FeNO concentration is less than or equal to about −3.0 ppb. In still another embodiment, after administering the pharmaceutical formulation, the subject's placebo-corrected change in FeNO concentration is less than or equal to about −10.0 ppb.

In an embodiment, after administering the pharmaceutical formulation, the subject's change in FeNO concentration is less than or equal to about −20.0 ppb. In another embodiment, after administering the pharmaceutical formulation, the subject's change in FeNO concentration is less than or equal to about −30.0 ppb. In yet another embodiment, after administering the pharmaceutical formulation, the subject's change in FeNO concentration is less than or equal to about −35.0 ppb. In still another embodiment, after administering the pharmaceutical formulation, the subject's in FeNO concentration is less than or equal to about −40.0 ppb.

In an embodiment, after administering the pharmaceutical formulation, the subject's placebo-corrected change in FeNO concentration is less than or equal to about −20.0 ppb. In another embodiment, after administering the pharmaceutical formulation, the subject's placebo-corrected change in FeNO concentration is less than or equal to about −30.0 ppb. In yet another embodiment, after administering the pharmaceutical formulation, the subject's placebo-corrected change in FeNO concentration is less than or equal to about −35.0 ppb. In still another embodiment, after administering the pharmaceutical formulation, the subject's placebo-corrected change in FeNO concentration is less than or equal to about −40.0 ppb.

In an embodiment, after administering the pharmaceutical formulation, the subject's change in FeNO concentration is less than or equal to about −45.0 ppb. In another embodiment, after administering the pharmaceutical formulation, the subject's average change in FeNO concentration is about −1.0 ppb to about −20.0 ppb. In yet another embodiment, after administering the pharmaceutical formulation, the subject's average change in FeNO concentration is about −5.0 ppb to about −15.0 ppb.

In an embodiment, after administering the pharmaceutical formulation, the subject's placebo-corrected change in FeNO concentration is less than or equal to about −45.0 ppb. In another embodiment, after administering the pharmaceutical formulation, the subject's average placebo-corrected change in FeNO concentration is about −1.0 ppb to about −20.0 ppb. In yet another embodiment, after administering the pharmaceutical formulation, the subject's average placebo-corrected change in FeNO concentration is about −5.0 ppb to about −15.0 ppb.

The Breathlessness, Cough, and Sputum Scale (BCSS) was developed to provide a quick and easy method of evaluating the severity of respiratory symptoms common in COPD patients (Semin. Respir. Crit. Care Med. 2009, 30, 629-359 and J. Aerosol Med. 2000, 13, 263-72). The BCSS is based on a three-item questionnaire assessing the patient's breathlessness, cough, and sputum.

In yet another embodiment, prior to administering the pharmaceutical formulation, the subject's breathlessness, cough, and sputum scale (BCSS) score is greater than or equal to 0. In still another embodiment, prior to administering the pharmaceutical formulation, the subject's BCSS score is greater than or equal to 1. In an embodiment, prior to administering the pharmaceutical formulation, the subject's BCSS score is greater than or equal to 3. In another embodiment, prior to administering the pharmaceutical formulation, the subject's BCSS score is greater than or equal to 6.

In yet another embodiment, prior to administering the pharmaceutical formulation, the subject's BCSS score is greater than or equal to 8. In still another embodiment, prior to administering the pharmaceutical formulation, the subject's average BCSS score is 3.

In an embodiment, the pharmaceutical formulation is administered at a dose that produces the below referenced exposures of JAK inhibitor as indicated by AUC_{0-∞}.

In an embodiment, administration of the pharmaceutical formulation produces an exposure of JAK inhibitor in the subject as indicated by AUC_{0-∞} that is greater than or equal to about 340,000 h·pg/mL. In another embodiment, administration of the pharmaceutical formulation produces an exposure of JAK inhibitor in the subject as indicated by AUC_{0-∞} that is greater than or equal to about 400,000 h·pg/mL. In yet another embodiment, administration of the pharmaceutical formulation produces an exposure of JAK inhibitor in the subject as indicated by AUC_{0-∞} that is greater than or equal to about 500,000 h·pg/mL.

In still another embodiment, administration of the pharmaceutical formulation produces an exposure of JAK inhibitor in the subject as indicated by AUC_{0-∞} that is greater than or equal to about 600,000 h·pg/mL. In an embodiment, administration of the pharmaceutical formulation produces an exposure of JAK inhibitor in the subject as indicated by AUC_{0-∞} that is greater than or equal to about 700,000 h·pg/mL. In another embodiment, administration of the pharmaceutical formulation produces an exposure of JAK inhibitor in the subject as indicated by AUC_{0-∞} that is greater than or equal to about 750,000 h·pg/mL.

In yet another embodiment, administration of the pharmaceutical formulation produces an exposure of JAK inhibitor in the subject as indicated by AUC_{0-∞} that is greater than or equal to about 755,000 h·pg/mL. In still another embodiment, wherein administration of the pharmaceutical formulation produces an average exposure of JAK inhibitor in the subject as indicated by AUC_{0-∞} that is about 555,000 h·pg/mL.

In an embodiment, the pharmaceutical formulation is administered at a dose that produces the below referenced exposures of JAK inhibitor as indicated by AUC_{0-last non-zero}.

In yet another embodiment, administration of the pharmaceutical formulation produces an exposure of JAK inhibitor in the subject as indicated by AUC_{0-last non-zero} that is greater than or equal to about 340,000 h·pg/mL. In still another embodiment, administration of the pharmaceutical formulation produces an exposure of JAK inhibitor in the subject as indicated by AUC_{0-last non-zero} that is greater than or equal to about 400,000 h·pg/mL. In an embodiment, administration of the pharmaceutical formulation produces an exposure of JAK inhibitor in the subject as indicated by AUC_{0-last non-zero} that is greater than or equal to about 500,000 h·pg/mL. In another embodiment, administration of the pharmaceutical formulation produces an exposure of JAK inhibitor in the subject as indicated by AUC_{0-last non-zero} that is greater than or equal to about 600,000 h·pg/mL.

In still another embodiment, administration of the pharmaceutical formulation produces an exposure of JAK inhibitor in the subject as indicated by AUC_{0-last non-zero} that is greater than or equal to about 700,000 h·pg/mL. In an embodiment, administration of the pharmaceutical formulation produces an exposure of JAK inhibitor in the subject as indicated by AUC_{0-last non-zero} that is greater than or equal to about 750,000 h·pg/mL. In another embodiment, administration of the pharmaceutical formulation produces an average exposure of JAK inhibitor in the subject as indicated by AUC_{0-last non-zero} that is about 555,000 h·pg/mL.

In an embodiment, the pharmaceutical formulation is administered at a dose that produces the below referenced exposures of JAK inhibitor as indicated by AUC_{over dosing interval}.

In yet another embodiment, administration of the pharmaceutical formulation produces an exposure of JAK inhibitor in the subject as indicated by AUC_{overdosing interval} that is greater than or equal to about 100,000 h·pg/mL. In still another embodiment, administration of the pharmaceutical formulation produces an exposure of JAK inhibitor in the subject as indicated by AUC_{overdosing interval} that is greater than or equal to about 130,000 h·pg/mL. In an embodiment, administration of the pharmaceutical formulation produces an exposure of JAK inhibitor in the subject as indicated by AUC_{overdosing interval} that is greater than or equal to about 200,000 h·pg/mL.

In another embodiment, administration of the pharmaceutical formulation produces an exposure of JAK inhibitor in the subject as indicated by AUC_{overdosing interval} that is greater than or equal to about 300,000 h·pg/mL. In yet another embodiment, administration of the pharmaceutical formulation produces an exposure of JAK inhibitor in the subject as indicated by AUC_{over dosing interval} that is greater than or equal to about 350,000 h·pg/mL. In still another embodiment, administration of the pharmaceutical formulation produces an exposure of JAK inhibitor in the subject as indicated by AUC_{overdosing interval} that is greater than or equal to about 370,000 h·pg/mL. In an embodiment, administration of the pharmaceutical formulation produces an average exposure of JAK inhibitor in the subject as indicated by AUC_{over dosing interval} that is about 272,000 h·pg/mL.

In an embodiment, the pharmaceutical formulation is administered at a dose that produces the below referenced exposures of JAK inhibitor as indicated by AUC_{0-12 hours}.

In another embodiment, administration of the pharmaceutical formulation produces an exposure of JAK inhibitor in the subject as indicated by AUC_{0-12 hours} that is greater than or equal to about 100,000 h·pg/mL. In yet another embodiment, administration of the pharmaceutical formulation produces an exposure of JAK inhibitor in the subject as indicated by AUC_{0-12 hours} that is greater than or equal to about 130,000 h·pg/mL.

In still another embodiment, administration of the pharmaceutical formulation produces an exposure of JAK inhibitor in the subject as indicated by AUC_{0-12 hours} that is greater than or equal to about 200,000 h·pg/mL. In an embodiment, administration of the pharmaceutical formulation produces an exposure of JAK inhibitor in the subject as indicated by AUC_{0-12 hours} that is greater than or equal to about 300,000 h·pg/mL. In another embodiment, administration of the pharmaceutical formulation produces an exposure of JAK inhibitor in the subject as indicated by AUC_{0-12 hours} that is greater than or equal to about 350,000 h·pg/mL.

In yet another embodiment, administration of the pharmaceutical formulation produces an exposure of JAK inhibitor in the subject as indicated by AUC_{0-12 hours} that is greater than or equal to about 370,000 h·pg/mL. In still another embodiment, administration of the pharmaceutical formulation produces an average exposure of JAK inhibitor in the subject as indicated by AUC_{0-12 hours} that is about 272,000 h·pg/mL.

In an embodiment, the pharmaceutical formulation is administered at a dose that produces the below referenced concentration of JAK inhibitor as indicated by Cₘₐₓ. In an embodiment, administration of the pharmaceutical formulation produces a concentration of JAK inhibitor in the subject as indicated by Cₘₐₓ that is greater than or equal to about 10,000 pg/mL. In another embodiment, administration of the pharmaceutical formulation produces a concentration of JAK inhibitor in the subject as indicated by Cₘₐₓ that is greater than or equal to about 12,250 pg/mL. In yet another embodiment, administration of the pharmaceutical formulation produces a concentration of JAK inhibitor in the subject as indicated by Cₘₐₓ that is greater than or equal to about 20,000 pg/mL. In still another embodiment, administration of the pharmaceutical formulation produces a concentration of JAK inhibitor in the subject as indicated by Cₘₐₓ that is greater than or equal to about 30,000 pg/mL.

In an embodiment, administration of the pharmaceutical formulation produces a concentration of JAK inhibitor in the subject as indicated by Cₘₐₓ that is greater than or equal to about 40,000 pg/mL. In another embodiment, administration of the pharmaceutical formulation produces a concentration of JAK inhibitor in the subject as indicated by Cₘₐₓ that is greater than or equal to about 43,000 pg/mL. In yet another embodiment, administration of the pharmaceutical formulation produces an average concentration of JAK inhibitor in the subject as indicated by Cₘₐₓ that is about 33,000 pg/mL.

In an embodiment, the pharmaceutical formulation is administered at a dose that produces the below referenced concentration of JAK inhibitor as indicated by C_{trough}. In still another embodiment, administration of the pharmaceutical formulation produces a concentration of JAK inhibitor in the subject as indicated by C_{trough} that is greater than or equal to about 9,000 pg/mL.

In an embodiment, administration of the pharmaceutical formulation produces a concentration of JAK inhibitor in the subject as indicated by C_{trough} that is greater than or equal to about 10,000 pg/mL. In another embodiment, administration of the pharmaceutical formulation produces a concentration of JAK inhibitor in the subject as indicated by C_{trough} that is greater than or equal to about 15,000 pg/mL. In yet another embodiment, administration of the pharmaceutical formulation produces a concentration of JAK inhibitor in the subject as indicated by C_{trough} that is greater than or equal to about 20,000 pg/mL. In still another embodiment, administration of the pharmaceutical formulation produces an average concentration of JAK inhibitor in the subject as indicated by C_{trough} that is about 14,000 pg/mL.

In an embodiment, the pharmaceutical formulation is administered at a dose that produces the below referenced half-life of JAK inhibitor as indicated by T_{1/2}.

In an embodiment, administration of the pharmaceutical formulation produces a half-life (T_{1/2}) of JAK inhibitor in the subject that is greater than or equal to about 15 hours. In another embodiment, administration of the pharmaceutical formulation produces a T_{1/2} of JAK inhibitor in the subject that is greater than or equal to about 17 hours. In yet another embodiment, administration of the pharmaceutical formulation produces a T_{1/2} of JAK inhibitor in the subject that is greater than or equal to about 20 hours. In still another embodiment, administration of the pharmaceutical formulation produces a T_{1/2} of JAK inhibitor in the subject that is greater than or equal to about 25 hours. In an embodiment, administration of the pharmaceutical formulation produces a T_{1/2} of JAK inhibitor in the subject that is greater than or equal to about 30 hours. In another embodiment, administration of the pharmaceutical formulation produces a T_{1/2} of JAK inhibitor in the subject that is greater than or equal to about 32 hours. In yet another embodiment, administration of the pharmaceutical formulation produces an average T_{1/2} of JAK inhibitor in the subject that is about 23 hours.

In an embodiment, the pharmaceutical formulation is administered at a dose that produces the above referenced pharmacokinetic parameters (AUC_{0-∞}, AUC_{0-last non-zero}, AUC_{over dosing interval}, AUC_{0-12 hours}, Cₘₐₓ, C_{trough}, and T_{1/2}).

In an embodiment, the above pharmacokinetic parameters can be measured 1, 2, 3, 4, 5, 6, 7, 8, 9, 10, 11, 12, 13, 14, 15, 16, 17, 18, 19, and/or 20 days after administration of the formulation.

Suitable doses for treating asthma and other respiratory disorders are expected to range from about 0.1 to about 100 mg/day of Compound I, including from about 0.2 to about 50 mg/day of active agent for an average 70 kg human. Suitable doses include, for example, about 0.001 mg/kg or 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, or 1 mg/kg.

In an embodiment, the subject is administered Compound I at any of the dosages referred to below.

In some embodiments, the subject is administered a formulation comprising Compound I at a dose of at least about 0.005 mg/kg. In some embodiments, the subject is administered a formulation comprising Compound I at a dose of from about 0.001 mg/kg to about 1 mg/kg. In some embodiments, the subject is administered a formulation comprising Compound I (e.g., a formulation comprising Compound I, MgSt, and lactose) at a dose of from about 0.1 mg/kg to about 1 mg/kg. In some embodiments, the subject is administered a formulation comprising Compound I at a dose of from about 0.01 mg/kg to about 0.1 mg/kg. In some embodiments, the subject is administered a formulation comprising Compound I (e.g., a formulation comprising Compound I, MgSt, and lactose) at a dose of 0.2 mg, 0.4 mg, 0.6 mg, 0.8 mg, 1.0 mg, 2.0 mg, 4.0 mg, 6.0 mg, 8.0 mg, 12.0 mg, 16.0 mg, 48.0 mg, or 68.0 mg. In some embodiments, the subject is administered a pharmaceutical formulation comprising Compound I (e.g., a pharmaceutical formulation comprising Compound I, MgSt, and lactose) at a dose of 0.2 mg, 0.6 mg, 2.0 mg, 4.0 mg, 6.0 mg, 8.0 mg, or 12.0 mg. In some embodiments, the subject is administered a pharmaceutical formulation comprising Compound I at a dose of about 0.2 mg. In some embodiments, the subject is administered a pharmaceutical formulation comprising Compound I at a dose of about 0.6 mg. In some embodiments, the subject is administered a pharmaceutical formulation comprising Compound I at a dose of about 2.0 mg. In some embodiments, the subject is administered a pharmaceutical formulation comprising Compound I at a dose of about 4.0 mg. In some embodiments, the subject is administered a pharmaceutical formulation comprising Compound I at a dose of about 6.0 mg. In some embodiments, the subject is administered a pharmaceutical formulation comprising Compound I at a dose of about 8.0 mg. In some embodiments, the subject is administered a pharmaceutical formulation comprising Compound I at a dose of about 12.0 mg.

In an embodiment, the subject is administered Compound I at any of the dosages referred to below.

In some embodiments, the subject is administered a pharmaceutical formulation comprising Compound I at any of the above doses once daily or twice daily. In some embodiments, the subject is administered a pharmaceutical formulation comprising Compound I at a dose of about 0.6 mg once daily. In some embodiments, the subject is administered a pharmaceutical formulation comprising Compound I at a dose of about 2.0 mg once daily. In some embodiments, the subject is administered a pharmaceutical formulation comprising Compound I at a dose of about 4.0 mg once daily. In some embodiments, the subject is administered a pharmaceutical formulation comprising Compound I at a dose of about 0.6 mg twice daily. In some embodiments, the subject is administered a pharmaceutical formulation comprising Compound I at a dose of about 2.0 mg twice daily. In some embodiments, the subject is administered a pharmaceutical formulation comprising Compound I at a dose of about 4.0 mg twice daily. In some embodiments, the subject is administered a pharmaceutical formulation comprising Compound I at a dose of about 8.0 mg once daily. In some embodiments, the subject is administered a pharmaceutical formulation comprising Compound I at a dose of about 8.0 mg twice daily. In some embodiments, the subject is administered a pharmaceutical formulation comprising Compound I at a dose of about 12.0 mg once daily.

In an embodiment of any of the methods disclosed herein, prior to administering the pharmaceutical formulation, the subject is undergoing treatment with a background therapy selected from an inhaled corticosteroid (ICS), an inhaled long-acting beta-agonist (LABA), and an inhaled long-acting muscarinic antagonist (LAMA), or a combination of two or more thereof. In another embodiment, prior to administering the pharmaceutical formulation, the subject is undergoing treatment with a LAMA, a LABA and a LAMA, or an ICS, a LABA, and a LAMA.

### EQUIVALENTS AND SCOPE

While various disclosure embodiments have been particularly shown and described in the present disclosure, it will be understood by those skilled in the art that various changes in form and details may be made without departing from the spirit and scope of the embodiments disclosed herein and set forth in the appended claims.

Those skilled in the art will recognize or be able to ascertain using no more than routine experimentation, many equivalents to the specific embodiments described herein. The scope of the present disclosure is not intended to be limited to the above description, but rather is as set forth in the appended claims.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

In addition, it is to be understood that any particular embodiment of the present disclosure that falls within the prior art may be explicitly excluded from any one or more of the claims. Since such embodiments are deemed to be known to those of ordinary skill in the art, they may be excluded even if the exclusion is not set forth explicitly herein. Any particular embodiments of compositions disclosed herein can be excluded from any one or more claims, for any reason, whether or not related to the existence of prior art.

All cited sources, for example, references, publications, databases, database entries, and art cited herein, are incorporated into this application by reference, even if not expressly stated in the citation. In case of conflicting statements of a cited source and the instant application, the statement in the instant application shall control.

### EXAMPLES

Compound I, (S)-3-(3-(1-methyl-2-oxo-5-(pyrazolo[1,5-a]pyridin-3-yl)-1,2-dihydro-3H-imidazo[4,5-b]pyridin-3-yl)piperidin-1-yl)-3-oxopropanenitrile, can be prepared according to the procedures outlined in WO 2011/051452 (see Example 7i) and WO 2016/124464 (see Form II, Example 2.2), both of which are incorporated herein by reference in their entireties. Any and all references to Compound I herein in the examples refer to polymorph Form II. Abbreviations as used herein have respective meanings as follows:

| | |
|---|---|
| API | Active pharmaceutical ingredient |
| AE | Adverse event(s) |
| APSD | Aerodynamic particle size distribution |
| AUC_{0-∞} | Area under the plasma concentration-time |
| | curve extrapolated to an infinite time |
| AUC_{0-last non-zero} | Area under the plasma concentration-time |
| | curve from dosing (time 0) to the time of the |
| | last measurable (non-zero) concentration |
| AUC_{0-12 hours} | Area under the plasma concentration-time |
| | curve from dosing (time 0) to 12 hours post- |
| | dose (time 12) |
| AUC_{0-24 hours} | Area under the plasma concentration-time |
| | curve over the last 24 h dosing interval |
| C_{trough} | Trough concentration of Compound I in the |
| | blood plasma prior to administration of a |
| | subsequent dose |
| Cₘₐₓ | Maximum concentration of Compound I in |
| | the blood plasma prior to administration of |
| | a subsequent dose |
| CSS | Croscarmellose sodium |
| F | Female |
| FeNO | Fractional exhaled nitric oxide |
| GMP | Good manufacturing practice |
| g | Gram(s) |
| T_{1/2} | Half-life |
| h or hr | Hour(s) |
| HPMC | Hydroxypropyl methylcellulose |
| ICS | Inhaled corticosteroid(s) |
| kg | Kilogram(s) |
| L | Liter(s) |
| L-HPC | Low-substituted hydroxypropyl cellulose |
| MGST or MgSt | Magnesium Stearate |
| M | Male |
| MoS | Margin of safety |
| MTD | Maximum tolerated dose |
| MCC | Microcrystalline Cellulose |
| mcg | Microgram(s) |
| mg | Milligram(s) |
| MMAD | Median mass aerodynamic diameter |
| μm or mcm | Micrometer(s) |
| MOC | Micro-orifice collector |
| min | Minute(s) |
| mL | Milliliter(s) |
| mm | Millimeter(s) |
| ng | Nanogram(s) |
| NGI | Next generation impactor |
| NHP | Nonhuman primate |
| NOAEL | No observed adverse effect level |
| OD/QD | Once daily |
| PK | Pharmacokinetics |
| pg | Picogram(s) |
| TK | Toxicokinetics |
| tox | Toxicology |
| TEAE | Treatment emergent adverse event(s) |
| TRV | Turbo rapid variable |
| BID | Twice daily |
| RSD | Relative standard deviation |
| rpm | Revolutions per minute |
| Std Dev | Standard deviation |
| vol | Volume |
| wt | Weight |
| wt % | Weight Percent |
| μL | Microliter(s) |

### General Procedure for Drug Content Determination

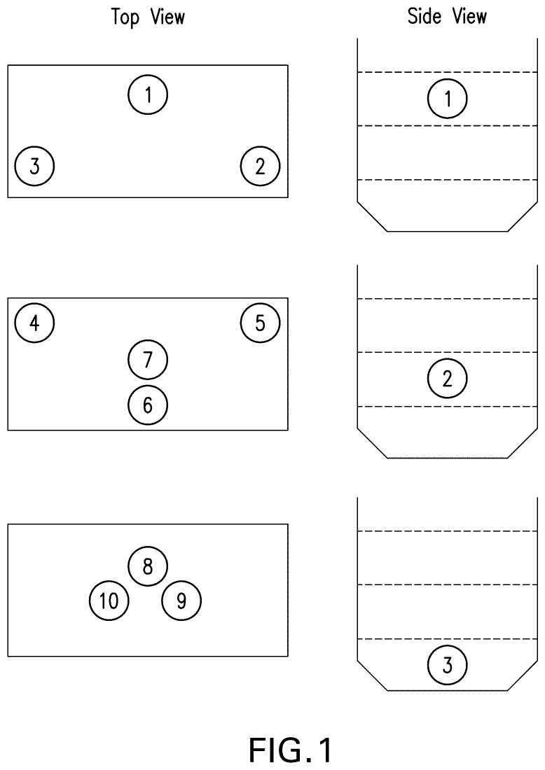

All drug content measurements were performed using location 7 as depicted in FIG. 1.

### 1 wt % Compound I Formulation

Approximately 300 mg of the formulation containing Compound I was transferred to a vial. The sample was transferred to a 500 mL volumetric flask and diluted to approximately 6 mcg/mL. Approximately 200 mL of the diluent was then sonicated for 10 minutes. The content of Compound I in the diluted sample was then determined by HPLC.

### 10 wt % Compound I Formulation

Approximately 400 mg of the formulation containing Compound I was transferred to a vial. The sample was transferred to a 500 mL volumetric flask and diluted to approximately 80 mcg/mL. Approximately 200 mL of the diluent was then sonicated for 10 minutes. Approximately 20 mL of the sonicated diluent was transferred to a 100 mL volumetric flask and diluted to an approximate concentration of 16 mcg/mL. The content of Compound I in the diluted sample was then determined by HPLC.

### General Procedure for Content Uniformity Determination

All content uniformity measurements were performed using locations 1-10 as depicted in FIG. 1.

### 1 wt % Compound I Formulation (Blend 1)

Approximately 60 mg of the formulation containing Compound I was transferred to a vial. The sample was transferred to a 100 mL volumetric flask and diluted to approximately 6 mcg/mL. Approximately 50 mL of the diluent was then sonicated for 10-20 minutes. The content uniformity of Compound I in the diluted sample was then determined by HPLC. This process was repeated for a total of 10 samples.

### 10 wt % Compound I Formulation (Blend 2 and 3)

Approximately 120 mg of the formulation containing Compound I was transferred to a vial. The sample was transferred to a 500 mL volumetric flask and diluted to approximately 24 mcg/mL. Approximately 250 mL of the diluent was then sonicated for 10-20 minutes. The content uniformity of Compound I in the diluted sample was then determined by HPLC. This process was repeated for a total of 10 samples.

### General Procedure for APSD, MMAD, FPM, and FPF Determination

### Cup Coating Agent Preparation

Cup coating agent was prepared by mixing 150 mL of a mixture of Brij® 35, ethanol, and glycerol with 850 mL of ethanol.

### NGI Procedure

The cup coating agent was prepared, and the NGI was assembled. The collection cups were then coated with cup coating agent and then proceeded to dry. A PALL® A/E 76 mm filter was placed into the back-up filter and attached to the NGI. Approximately 15 mL of diluent was added into the pre-separator insert. The flow rate was set to 90 L/min and the capsule was pierced. One dose was discharged into the NGI by initiating the time-controlled solenoid valve for 2.70 seconds. Sample diluent was added (10 mL for 0.2 mg and 2.0 mg doses and 20 mL for 4.0 mg doses), and the collection cups were agitated on the NGI Gentle Rocker for 10 minutes. The induction port was added 40 mL of sample diluent and was shaken for 1 minute. The pre-separator was added 50 mL of sample diluent and was shaken for 1 minute. The back-up filter was transferred to a crystallizing dish with 20 mL of sample diluent and was then sonicated for 5 minutes. The capsule from each determination was then transferred to a 20 mL volumetric flask and sample diluent was added. The suspension was then shaken and sonicated until fully dissolved. The resulting solution equilibrated under ambient conditions and was then made up to volume. Each Monodose RS01 was placed into an individual plastic bag and 20 mL of recovery solution was added. The bag was shaken by hand for 1 minute and filtered prior to HPLC analysis.

### General Procedure for Compound I Delivered Dose Determination

A Castellated fixed volume dosage unit sampling apparatus (DUSA) was assembled and attached to a backup filter. PALL® A/E 47 mm filter papers were used in the DUSA and PALL® A/E 76 mm filter were used in the backup filter. The solenoid timer was then equipped and the flow rate was adjusted to 90 L/min. The pre-sample weight of the device was recorded and the inhaler was activated. The dose was discharged twice into the DUSA by initiating the timer controlled solenoid valve. The post-sample weight of the device was then recorded and the exhaust bung was inserted into the exhaust port. Sample diluent (50 mL) was intrudced into the DUSA through the mouthpiece adaptor, and the DUSA was placed on orbital shaker for 5 minutes at 250 shakes per minute. The capsule is dissolved in diluent and drug retained on the device is recovered by washing with diluent. Dilutions were performed with 4 mg capsules. All samples were filtered through a disposable 0.2 μm PTFE syringeless filter with polypropylene housing into HPLC vials and analyzed via HPLC.

### Example 1: Low-Strength Formulation of Compound I (0.2 mg)

Blend 1 (with Co-Milling) (0.4 kg Scale)

A 0.9 wt % magnesium stearate excipient preblend, i.e., lactose:MgSt, was prepared by combining lactose, 10% fines (3962.4 g) with magnesium stearate (37.6 g). A 6.5 wt % magnesium stearate API preblend, i.e., API:MgSt, was prepared by combining Compound I (69.9 g) with magnesium stearate (4.9 g). In a subsequent step, a U5 Comil® with a 457 mcm sieve was used to process 395.1 g of the lactose:MgSt preblend and 4.8 g of the API:MgSt preblend at 1000 rpm into a 1 L TRV bowl. The resulting mixture was then homogenized in a TRV blender at 1209 rpm for 4 minutes to afford a 1 wt % API bulk powder formulation (Blend 1). The Blend 1 bulk powder was transferred to a clean, sealed GMP stainless-steel container (160 mm×160 mm) which followed with a resting period (20 hours, 40 hours, or 60 hours) prior to capsule filling. Capsules were hand-filled with 20 mg of the Blend 1 bulk powder for a 0.2 mg target dose.

The resulting composition of the 0.2 mg product is shown in Table 1.

TABLE 1

| Component | Amount (mg) | Composition (wt %) |
|---|---|---|
| Compound I | 0.2 | 1.1 |
| Magnesium Stearate | 0.2 | 1.0 |
| Lactose | 19.6 | 97.9 |

Content uniformity and drug content as determined for the Blend 1 bulk powder along with aerodynamic particle size distribution as determined for the Blend 1 capsules is provided in Table 2.

TABLE 2

| | | Blend 1 | | |
|---|---|---|---|---|
| | Acceptance | (Comil) | | |
| Test | Criteria | 20 Hr | 40 Hr | 60 Hr |
| Content | ≤5.0% RSD | 1.4 | 1.4 | 2.0 |
| Uniformity | | | | |
| Drug | 0.85-1.15 | 1.07 | 1.07 | 1.07 |
| Content | wt % | | | |
| APSD | Mean Total | 94.1 | 99.1 | 101.4 |
| | Recovery as % | | | |
| | of label claim | | | |
| | Mean Fine | 62.9 | 58.4 | 60.5 |
| | Particle | | | |
| | Fraction (%) | | | |

TABLE 3

| | 20 Hr | 40 Hr | 60 Hr |
|---|---|---|---|
| | Blend 1 | Blend 1 | Blend 1 |
| Impactor stage | Std dev | Std dev | Std dev |
| Device | 3.7 | 11.6 | 2.3 |
| Capsule | 0.8 | 0.8 | 1.7 |
| Induction Port | 2.4 | 3.1 | 2.3 |
| Pre-Separator | 1.3 | 1.7 | 1.3 |
| Stage 1 | 1.1 | 0.6 | 0.5 |
| Stage 2 | 2.9 | 0.5 | 1.0 |
| Stage 3 | 1.6 | 0.5 | 1.0 |
| Stage 4 | 2.9 | 1.0 | 0.9 |
| Stage 5 | 1.3 | 0.5 | 0.7 |
| Stage 6 | 0.6 | 0.4 | 0.4 |
| Stage 7 | 0.6 | 0.2 | 0.3 |
| MOC | 0.4 | 0.2 | 0.1 |

The results from Table 3 show that a decrease in variability was observed after rest periods of 40 and 60 hours compared to a rest period of 20 hours for Blend 1.

### Example 2: High-Strength Formulations of Compound I (2.0 mg and 4.0 mg)

### Blend 2 (0.4 kg Scale)

A 0.6 wt % magnesium stearate excipient preblend, i.e., lactose:MgSt, was prepared by combining lactose, 6% fines (3976.8 g) with magnesium stearate (23.3 g). A 6.5 wt % magnesium stearate API preblend, i.e., API:MgSt, was prepared by combining Compound I (69.9 g) with magnesium stearate (4.9 g). In a subsequent step, a U5 Comil® with a 457 mcm sieve was used to process 356.0 g of the lactose:MgSt preblend and 44.0 g of the API:MgSt preblend at 1000 rpm into a 1 L TRV bowl to produce a mixture. The resulting mixture was then homogenized in a TRV blender at 2392 rpm for 4 minutes to afford a 10 wt % API bulk powder formulation (Blend 2). The Blend 2 bulk powder was then transferred to a clean, sealed GMP stainless-steel container (160 mm×160 mm) which followed with a resting period (20 hours or 60 hours) prior to capsule filling. Size 3 HPMC capsules were hand-filled with 20 mg of the Blend 2 bulk powder for a 2.0 mg target dose and 40 mg of the Blend 2 bulk powder for a 4.0 mg target dose. The resulting composition of the 2.0 mg and 4.0 mg products is shown in Table 4.

TABLE 4

| | Amount (mg) | Amount (mg) | Composition |
|---|---|---|---|
| Component | 20 mg Capsule | 40 mg Capsule | (wt %) |
| Compound I | 2.1 | 4.1 | 10.3 |
| Magnesium Stearate | 0.3 | 0.5 | 1.2 |
| Lactose | 17.7 | 35.4 | 88.5 |

Content uniformity, drug content, and aerodynamic particle size distribution as determined for the bulk powder, 20 mg capsule, and 40 mg capsule are provided in Table 5.

TABLE 5

| | | | | 20 mg Filled | | 40 mg Filled | |
|---|---|---|---|---|---|---|---|
| | | | | Capsules | | Capsules | |
| | | Bulk | | (2.0 mg | | (4.0 mg | |
| | Acceptance | Powder | | Target Dose) | | Target Dose) | |
| Test | Criteria | 20 Hr | 60 Hr | 20 Hr | 60 Hr | 20 Hr | 60 Hr |
| Content | ≤5.0% RSD | 1.6 | 0.2 | | | | |
| Uniformity | | | | | | | |
| Drug | 8.5-11.5 | 9.6 | 9.5 | | | | |
| Content | wt % | | | | | | |
| APSD | Mean Total | | | 89.7 | 85.4 | 86.1 | 81.9 |
| | Recovery | | | | | | |
| | as % of | | | | | | |
| | label claim | | | | | | |
| | Mean Fine | | | 46.2 | 56.7 | 49.8 | 52.9 |
| | Particle | | | | | | |
| | Fraction | | | | | | |
| | (%) | | | | | | |

Improved homogeneity of the Blend 2 bulk powder was observed after a rest period of 60 hours compared to a rest period of 20 hours as noted by a decrease in RSD. In addition, an increase in the mean fine particle fraction was observed after a rest period of 60 hours compared to a rest period of 20 hours for both the 20 mg (2.0 mg of Compound I) and 40 mg (4.0 mg of Compound I) capsules filled with Blend 2.

Blend 3 (2 kg scale)

A 0.6 wt % magnesium stearate excipient preblend, i.e., lactose:MgSt, was prepared by combining lactose, 6% fines (3976.6 g) with magnesium stearate (23.2 g). A 6.5 wt % magnesium stearate API preblend, i.e., API:MgSt, was prepared by combining Compound I (243.3 g) with magnesium stearate (16.9 g). In a subsequent step, a U5 Comil® with a 457 mcm sieve was used to process 1792.3 g of the lactose:MgSt preblend and 175.9 g of the API:MgSt preblend at 1000 rpm into a 5 L TRV bowl to produce a mixture. The resulting mixture was then homogenized in a TRV blender at 1385 rpm for 7 minutes to afford a 10 wt % API bulk powder formulation (Blend 3). The Blend 3 bulk powder was then transferred to a clean, sealed GMP stainless-steel container (160 mm×160 mm) which followed with a resting period of 60 hours prior to capsule filling. Size 3 HPMC capsules were hand-filled with 20 mg of the Blend 3 bulk powder for a 2.0 mg target dose and 40 mg of the Blend 3 bulk powder for a 4.0 mg target dose.

The resulting composition of the 2.0 mg and 4.0 mg product is shown in Table 6.

TABLE 6

| Component | Composition (wt %) |
|---|---|
| Compound I | 10.3 |
| Magnesium Stearate | 1.2 |
| Lactose | 88.5 |

Content uniformity and drug content are provided in Table 7.

TABLE 7

| Test | Acceptance Criteria | Result |
|---|---|---|
| Content Uniformity | ≤5.0% RSD | 0.7 |
| Drug Content | 8.5-11.5 wt % | 8.9 |

### Example 3: Phase 1 Part 4 Clinical Trials of Formulations of Compound I (4 mg BID)

### Formulations Comprising Compound I, Lactose, and Magnesium Stearate

Capsules filled with blend 3 were used to achieve a 4.0 mg target dose of Compound I (4.0 mg capsule×1) used in Part 4 clinical studies. Twice daily dosing (BID) was achieved by repeating the preceding regime. For example, a 4.0 mg BID dosing regime was achieved by administering a single 4.0 mg capsule in the morning and a single 4.0 mg capsule in the evening.

### COPD Assessment Test (CAT)

The COPD Assessment Test (CAT) is a validated, short (8 item) and simple, patient completed questionnaire. It was developed to measure the health statues of patients with COPD. The 8 items which are included in the CAT cover cough, phlegm, chest tightness, breathlessness going up hills/stairs, activity limitation at home, confidence leaving home, sleep and energy. Studies have shown the questionnaire to be responsive to change and to treatment (Eur. Respir. J., 2009, 34, 648-654).

A CAT questionnaire was read to the subject and the responses (i.e., score) were documented accordingly by a clinical study team member.

Modified Medical Research Council (mMRC) Dyspnea Questionnaire

All of the statements (Grade 0 to Grade 4) were read to the subject and the responses (i.e., Grade) were documented accordingly by a clinical study team member.

Each subject was asked the following:
Which of these statements best describes your breathing?
   Grade 0: Not troubled by breathlessness except with strenuous exercise
   Grade 1: Troubled by shortness of breath when hurrying on the level or walking up a slight hill
   Grade 2: Walks slower than people of the same age on the level because of breathlessness or has to stop for breath when walking at own pace on the level
   Grade 3: Stops to breathe after walking about 100 yards (meters) or after a few minutes on the level
   Grade 4: Too breathless to leave the house or breathless when dressing or undressing

According to the mMRC scale, Grade 0 is the lowest severity of dyspnea and Grade 4 is the highest severity of dyspnea (BMJ, 1960, 2, 1662).

### Breathlessness, Cough, and Sputum Scale (BCSS)

The Breathlessness, Cough and Sputum Scale (BCSS) is a patient-reported outcome measurement to evaluate symptoms in patients with chronic obstructive pulmonary disease (COPD) (Chest, 2003, 124, 2132-2191). Subjects were asked to rate the severity of the three symptoms over a 24-hour period, each on a 5-point scale. Higher scores indicate more severe symptoms. Item scores were summed to yield a total score. The BCSS questionnaire score referenced on a particular day is a patient's perception of their symptoms from the previous 24-hour period.

The questionnaire allows subjects to record symptoms on a 5-point Likert scale from 0 (no symptoms) to 4 (severe symptoms) (J. Aerosol Med. 2000, 13, 263-72). The results from the questionnaire are reported as the sum of responses to the three questions. The BCSS easily shows the patient's perception of COPD symptoms.

A sample BCSS questionnaire is below:
1) How much difficulty did you have breathing today?
   0=None—unaware of any difficulty
   1=Mild—noticeable when performing strenuous activity (e.g. running)
   2=Moderate—noticeable even when performing light activity (e.g. bedmaking or carrying groceries)
   3=Marked—noticeable when washing or dressing
   4=Severe—almost constant, present even when resting
2) How was your cough today?
   0=No cough—unaware of coughing
   1=Rare—cough now and then
   2=Occasional—less than hourly
   3=Frequent—one or more times an hour
   4=Almost constant—never free of cough or need to cough
3) How much trouble did you have due to sputum today?
   0=None—unaware of any trouble
   1=Mild—rarely caused trouble
   2=Moderate—noticeable trouble
   3=Marked—caused a great deal of trouble
   4=Severe—almost constant trouble

### Spirometry (FEV1, FVC):

Spirometry was performed according to the ATS/ERS 2019 guidelines (Am. J. Respir. Crit. Care Med., 2019, 200, e70-e88). Spirometry was performed using NDD TrueFlow spirometers. These spirometers were factory calibrated and were maintained per manufacturer instructions.

Pre-bronchodilator spirometry (for pre- and post-bronchodilator eligibility spirometry as appropriate only) was performed in the absence of SABA and SAMA, i.e. neither SABA nor SAMA were used in the preceding 6-hour period. However, for subjects that required a SABA or SAMA inhaler prior to the screening visit due to their asthma symptoms, then the pre-BD spirometry was delayed or the visit rescheduled. In addition, maintenance medications containing a LABA or a LAMA were postponed until the pre and post BD spirometry test for eligibility was completed. Restrictions on SABA or SAMA use were only required for pre- and post-bronchodilator spirometry at screening (e.g., eligibility).

Predicted normal values were based on the Global Lung Function Initiative predicted values (Eur. Respir. J., 2012, 40, 1324-1343).

### Fractional Exhaled Nitric Oxide (FeNO)

FeNO measurements were performed according to the ATS/ERS 2005 guidelines (Am. J. Respir. Crit. Care Med., 2005, 171, 912-930) using an MCG Diagnostics FeNO+ instrument. A minimum of two measurements were performed and the average of the two closest values was reported.

The below reproducibility criteria were used:
If the higher of the two selected values was 30 parts per billion (ppb), then the two measurements should be within 10% of each other (i.e., lower value within 10% of the higher value).
If the higher of the two selected values is below 30 ppb, then the two measurements should be within 3 ppb of each other.

Additional measurements were performed (up to a maximum of 8) until the above reproducibility criteria were met.

Food and water were restricted for 1 hour prior to FeNO measurements. Nitrate rich foods were restricted for 6 hours prior to FeNO measurements (e.g. vegetable juices, salads, lettuce, radishes, celery, broccoli, cauliflower, spinach, rocket, beets, parsley, leeks, cabbage, fennel, turnips, carrots, cured meats, bacon, and carbonated drinks).

If the above inclusion criteria were not met at screening, a repeat session was performed after an interval of at least 15 minutes, or a repeat screening was scheduled for another day.

The below inclusion criteria were used:
FeNO must be ≥30 ppb at screening and prior to randomization on Day −1 or Day 1.

If the subject's FeNO was <30 ppb at Day −1, a repeat was performed after at least a 15-minute interval. If the repeated FeNO measurement was also <30 ppb, the subject must achieve the inclusion criteria on Day 1 prior to randomization. If required, a repeat was made after at least a 15-minute interval, prior to randomization.

### Plasma Sample Collection and Processing

The appropriate kit was selected and the subject's ID and required information was recorded. At the nominated time-point, 4 mL of blood were drawn into an appropriately labelled K2EDTA Vacuette. The tube was then gently inverted 4 to 6 times. The date and time were then recorded on the sample requisition form. The whole blood sample was stored at ambient temperature until processing (maximum of 4 hours).

The whole blood sample was then transferred to a centrifuge set at +4° C. and the sample was centrifuged at 2000 g for 10 min at +4° C. A pipette was then used to transfer the resultant plasma (top layer) into two appropriately labelled transfer vials (approximately 1 mL of plasma each). The K2EDTA Vacuette and remaining whole blood sample was then discarded and the plasma samples were stored at −20° C. or below until shipment.

### Bioanalytical Methods for PK Studies

Plasma samples were analyzed for pharmacokinetic purposes according to the method below in Table 8 and Table 9 (LC system and gradient profile, respectively). The method was validated according to internationally accepted standards. Mass spectrometry conditions are shown in Table 10 and Table 11 (mass spectrometer and MRM transitions, respectively).

TABLE 8

| | |
|---|---|
| System | Waters Acquity |
| Run Time | 3.3 min |
| Mobile phase A | 0.1% Formic Acid (ACN) |
| Mobile phase B | B 0.1% Formic Acid (aq) |
| Injection mode | Partial loop |
| Weak wash | ACN:H₂O (20:80, v/v) |
| Weak wash volume | 1500 μL |
| Strong wash | ACN:H₂O:IPA:FA (40:30:30:0.1. v/v/v/W) |
| Strong wash volume | 1000 UL |
| Analytical Column | Waters Acquity BEH C18, 50 × 2.1 mm, |
| | 1.7 μm |
| Column temperature | Nominally +40° C. |
| Autosampler | Nominally +4° C. |
| temperature | |

TABLE 9

| Time | Flow Rate | | | |
|---|---|---|---|---|
| (mins) | (mL/min) | A (%) | B (%) | Curve |
| Initial | 0.600 | 10 | 90 | — |
| 0.2 | 0.600 | 10 | 90 | 6 |
| 1.6 | 0.600 | 60 | 40 | 6 |
| 1.7 | 0.600 | 100 | 0 | 6 |
| 2.7 | 0.600 | 100 | 0 | 6 |
| 2.8 | 0.600 | 10 | 90 | 6 |
| 3.3 | 0.600 | 10 | 90 | 6 |
| 10.0^{a} | 0.600 | 100 | 0 | 11 |

^{a}Post run column wash only

TABLE 10

| System | Sciex API 5000 | |
|---|---|---|
| Ionization/Interface | Turbo Spray | |
| Collision Gas (CAD) | 7 | psi |
| Curtain Gas (CUR) | 30 | psi |
| Gas 1 (GS1) | 30 | psi |
| Gas 2 (GS2) | 70 | psi |
| Ionspray Voltage (IS) | 5500 | V |
| Source Temperature (TEM) | +650° | C. |

TABLE 11

| | Precursor | Product | Dwell | | | Typical |
|---|---|---|---|---|---|---|
| | ion^{a} | ion^{a} | Time | | | R.T. |
| Analyte/IS | (m/z) | (m/z) | (ms) | Res. | Polarity | (min) |
| 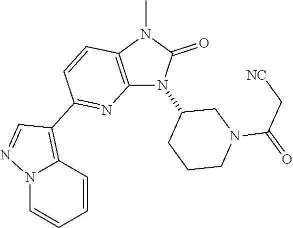 | 416 | 349 | 50 | Unit | Positive | 1.4 |
| Compound I | | | | | | |
| 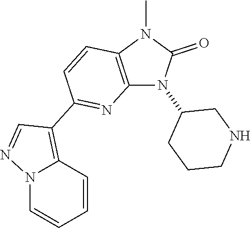 | 349 | 266 | 50 | Unit | Positive | 1.0 |
| Compound II | | | | | | |
| (main metabolite of | | | | | | |
| Compound I quantified in | | | | | | |
| plasma) | | | | | | |
| 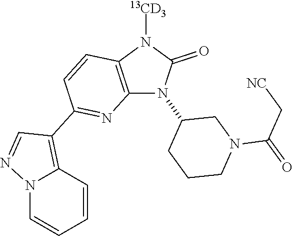 | 420 | 270 | 50 | Unit | Positive | 1.4 |
| Compound III | | | | | | |
| (Compound I | | | | | | |
| Internal Standard) | | | | | | |
| 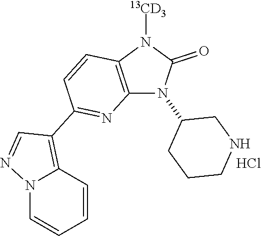 | 353 | 270 | 50 | Unit | Positive | 1.0 |
| Compound IV | | | | | | |
| (Compound II | | | | | | |
| Internal Standard) | | | | | | |

^{a}Masses for Precursor/Product ions were nominal.

Baseline Patient Population (n=13)

Lactose/MgSt-based formulations of Compound I were evaluated in a specific COP population in Part 4 of the Phase 1 clinical trials (NCT05006521). The COPD population is characterized by the metrics outlined in Table 12.

Subjects were to have stable treatment with at least one maintenance inhaler (e.g., LAMA, LABA, and ICS taken by mono product or combination inhalers) for at least three months prior to screening. All enrolled subjects were using ICS, and/or LABA, and/or LAMA mono- or combination background therapy. Among all 13 patients, all but one patient (on a LAMA) were using either triple or double combination background treatment (i.e., LABA/LAMA or ICS/LABA/LAMA). Each active subject was administered 4 mg of Compound I BID (n=9). Four subjects were administered a placebo BID.

TABLE 12

| | | |
|---|---|---|
| Parameter | Mean | Range |

Pre-bronchodilator

| | | |
|---|---|---|
| FEV₁ % of predicted normal¹ | 59.9 | 34-77 |
| FEV₁ (L) | 1.7 | 0.9-2.4 |
| FVC (L) | 3.3 | 2.1-4.3 |
| FVC % of predicted normal¹ | 91.2 | 70-111 |
| FEV₁/FVC | 0.507 | 0.378-0.593 |
| FEF₂₅₋₇₅ (L/sec) | 0.6 | 0.3-1.2 |

Post-bronchodilator

| FEV₁ % of predicted normal¹ | 69.8 | 52-86 |
|---|---|---|
| FEV₁ (L) | 2.0 | 1.1-3.0 |
| FVC (L) | 3.6 | 2.4-5.1 |
| FVC % of predicted normal¹ | 98.8 | 80-127 |
| FEV₁/FVC | 0.548 | 0.46-0.66 |
| FEF₂₅₋₇₅ (L/sec) | Not reported | |
| Blood eosinophil count (cells/μL) | 235 | 70-480 |
| Fractional exhaled nitric oxide (ppb) | 29.6 | 6.1-70.8 |
| COPD assessment test (CAT) score (n = 9 active) | 15 | 4-30 |
| modified Medical Research Council (mMRC) | | |
| Dyspnea Questionnaire Grades (n = 9 active) | | |
| Grade 0 | 1 | |
| Grade 1 | 4 | |
| Grade 2 | 3 | |
| Grade 3 | 0 | |
| Grade 4 | 1 | |
| Breathlessness Cough and Sputum Scale (BCSS) | 3 | 1-8 |
| Score (n = 9 active) | | |

¹Based on predicted normal values using Global Lung Index, GLI 2012, predicted values.

Compound I (4 mg) was administered twice-daily on Day 1 to 9 with a single 4 mg dose administered on the morning of Day 10 followed by a two-day (Days 11-12) no treatment period and a follow-up five days later (Day 17). Adverse Events (AEs) were recorded at each visit with hematology/chemistry and PK blood samples collected on multiple occasions during the 17-day period.

### Pharmacokinetic Study Summary

Day 10 pharmacokinetic parameters for Part 4 of Phase 1 are summarized below in Table 13.

TABLE 13

| | Day 10 Mean | Day 10 Range |
|---|---|---|
| Parameter | (n = 9) | (n = 9) |
| AUC_{0-∞} (h · pg/mL) | 554,819 | 347,430 to 754,242 |
| AUC_{0-lastnon-zero} (h · pg/mL) | 556,927 | 346,769 to 750,840 |
| AUC_{over dosing interval} (h · pg/mL) | 272,240 | 132,370 to 370,059 |
| AUC_{0-12 hours} (h · pg/mL) | 272,240 | 132,370 to 370,059 |
| Cₘₐₓ (pg/mL) | 32,956 | 12,400 to 42,900 |
| T_{1/2} (hours) | 23.3 | 17.0 to 31.2 |
| C_{trough} (pg/mL) | 13,921 | 9,628 to 19,700 |

Thirteen subjects (9 Compound I: 4 placebo) received treatment. No serious AEs were reported and no subjects withdrew due to an AE. Eleven subjects (53.8%) experienced AEs; 5 (55.6%) Compound I and 2 (50.0%) placebo. The most common AE was headache; 2 subjects [22.2%] Compound I and 1 subject [25.0%] placebo. Safety lab assessments did not reveal findings of clinical concern for formulations comprising Compound I.

Steady state PK was achieved by Day 3 with minimal accumulation (accumulation ratio <2) upon repeat dosing. Plasma levels were below pharmacologically active concentrations. The Compound I safety/PK profile was consistent with that reported in subjects with mild and moderate to severe asthma (WO 2024/119058, incorporated herein by reference in its entirety). Thus, inhaled Compound I was well tolerated by subjects with COPD on background therapy.

### Placebo-Corrected Changes in FeNO

Placebo-corrected changes in FeNO are reported below in Table 14 for COPD patients treated with Compound I (4 mg BID). Subjects were to have stable treatment with at least one maintenance inhaler (e.g., LAMA, LABA, ICS taken by mono product or combination inhalers) for at least three months prior to screening.

TABLE 14

| | Day 10 Mean % | Day 10 Mean ppb |
|---|---|---|
| Timepoint | change (range) | change (range) |
| Day 10 pre-dose | −38.25 (−54.9 to −11.4) | −12.56 (−31.8 to −3.1) |
| Day 10 + 3 hours | −39.95 (−85.5 to 3.6) | −8.01 (−36.4 to 11.4) |
| Day 10 + 6 hours | −40.70 (−85.6 to 13.0) | −9.11 (−41.2 to 13.0) |
| Day 10 + 12 hours | −42.29 (−84.4 to 3.9) | −13.45 (−43.9 to 0.9) |
| Day 10 mean | −39.97 (−77.6 to −10.1) | −10.52 (−38.3 to −1.5) |

As can be seen from Table 14, placebo-corrected FeNO reductions ≥20% were achieved in subjects independent of baseline FeNO and blood eosinophil levels. Additionally, mean placebo-corrected FeNO reductions ≥20% were maintained after a 2-day and a 7-day no dosing period.

For comparison, it is established that FeNO reductions greater than 20% are clinically significant (Am. J. Respir. Crit. Care Med. 2011, 184, 602). The results suggest that clinically relevant FeNO reductions are achieved in COPD populations post administration of a 4 mg BID regimen of formulations comprising Compound I who also use ICS, and/or LABA, and/or LAMA mono- or combination background therapy.

### Placebo-Corrected Changes in FEV₁

Placebo-corrected changes in FEV₁ are reported below in Table 15 for COPD patients treated with Compound I (4 mg BID). Subjects were to have stable treatment with at least one maintenance inhaler (e.g., LAMA, LABA, ICS taken by mono product or combination inhalers) for at least three months prior to screening.

TABLE 15

| Timepoint | Day 10 Mean (mL) (n = 9) | Day 10 Range (mL) |
|---|---|---|
| Day 10 pre-dose | 140 | +410 to −200 |
| Day 10 + 0.5 hours | 190 | +390 to −40 |
| Day 10 + 1 hours | 210 | +570 to −300 |
| Day 10 + 2 hours | 180 | +410 to −200 |
| Day 10 + 4 hours | 200 | +420 to −220 |
| Day 10 + 8 hours | 240 | +650 to −70 |

Table 15 shows that increases in FEV₁ were achieved post-administration of Compound I (4.0 mg BID) as indicated by the Day 10 average FEV₁ data. This corresponds to an improvement in FEV₁ in subjects independent of baseline FeNO and blood eosinophil levels. This level of improvement in trough FEV₁ is consistent with in clinical trial data of FDA approved bronchodilators and ICS/LABA combinations for COPD (COPD, 2005, 2, 111-124).

### Example 4: Phase 1 Part 4 Continued—Subjects with COPD Characterized by T2 Low and T2 High Inflammation

### Formulations Comprising Compound I, Lactose, and Magnesium Stearate

Capsules filled with blend 3 were used to achieve a 4.0 mg target dose of Compound I (4.0 mg capsule×1) used in Part 4 clinical studies. Twice daily dosing (BID) was achieved by repeating the preceding regime. For example, a 4.0 mg BID dosing regime was achieved by administering a single 4.0 mg capsule in the morning and a single 4.0 mg capsule in the evening.

Patients with moderate to severe COPD that participated in Part 4 of the Phase 1 clinical study (Table 12) were not required to have an elevated FeNO at study entry. Patients were required to have a blood eosinophil count of >50 cells/μL. Consequently, the majority of the cohort had blood eosinophil levels consistent with T2 low inflammation (i.e., <300 cells/μL) allowing an evaluation of changes in lung function (e.g., FEV₁) in subjects with T2 low inflammation. In addition, changes in lung function and FeNO levels on treatment with Compound I were compared between subjects with T2 low inflammation and subjects with subjects with T2 high inflammation.

Nine patients were randomized to 4 mg Compound I BID active treatment. Among the 9 active patients, all but one patient (on a LAMA) was using triple therapy. The majority of the cohort (67% or 6/9) had T2 low inflammation as defined by blood eosinophils <300 cells/μL on Day 1 pre-dose. The COPD population is characterized by the metrics outlined in Table 12.

### Placebo-Corrected Changes in FEV₁ (Overall COPD Population)

Placebo-corrected changes in FEV₁ (post-bronchodilator) are reported below in Table 16 for COPD patients treated with Compound I (4 mg BID).

TABLE 16

| Timepoint | Day 10 Mean (%) (n = 9) | Max improvement (%) |
|---|---|---|
| Day 10 pre-dose | +5.8 | +15.5 |
| Day 10 + 0.5 hours | +7.9 | +16.3 |
| Day 10 + 1 hours | +9.5 | +13.5 |
| Day 10 + 2 hours | +7.1 | +13.3 |
| Day 10 + 4 hours | +8.4 | +16.8 |
| Day 10 + 8 hours | +10.3 | +24.8 |

Table 16 shows that increases in FEV₁ were achieved post-administration of Compound I (4.0 mg BID) as indicated by the Day 10 average FEV₁ data. This corresponds to an improvement in FEV₁ in subjects independent of baseline FeNO levels.

### Placebo-Corrected Changes in FVC (Overall COPD Population)

Placebo-corrected changes in FVC (post-bronchodilator) are reported below in Table 17 (L) and Table 18 (%) for COPD patients treated with Compound I (4 mg BID).

TABLE 17

| | Mean (mL) | Max improvement |
|---|---|---|
| Timepoint | (n = 9) | (mL) |
| Day 10 pre-dose | −30 | +423 |
| Day 10 + 0.5 hours | −55 | +375 |
| Day 10 + 1 hours | +210 | +1130 |
| Day 10 + 2 hours | +72 | +408 |
| Day 10 + 4 hours | +92 | +565 |
| Day 10 + 8 hours | +162 | +630 |

TABLE 18

| Timepoint | Mean (%) (n = 9) | Max improvement (%) |
|---|---|---|
| Day 10 pre-dose | +0.3 | +14 |
| Day 10 + 0.5 hours | +0.2 | +13.5 |
| Day 10 + 1 hours | +5.5 | +18.8 |
| Day 10 + 2 hours | +2.5 | +9.8 |
| Day 10 + 4 hours | +4.1 | +17.8 |
| Day 10 + 8 hours | +6.6 | +18.3 |

Tables 17 and 18 show that improvements in FVC were achieved post-administration of Compound I (4.0 mg BID) as indicated by the Day 10 FVC data. This corresponds to an improvement in FVC in subjects independent of baseline FeNO levels.

### Placebo-Corrected Changes in FEV₁/FVC (Overall COPD Population)

Placebo-corrected changes in FEV₁/FVC (post-bronchodilator) are reported below in Table 19 for COPD patients treated with Compound I (4 mg BID).

TABLE 19

| Timepoint | Mean (%) (n = 9) | Max improvement (%) |
|---|---|---|
| Day 10 pre-dose | +3.81 | +6.85 |
| Day 10 + 0.5 hours | +5.98 | +8.78 |
| Day 10 + 1 hours | +2.34 | +6.05 |
| Day 10 + 2 hours | +3.83 | +7.35 |
| Day 10 + 4 hours | +3.91 | +7.38 |
| Day 10 + 8 hours | +4.01 | +8.15 |

As can be seen in Table 19, improvements in FEV₁/FVC were achieved post-administration of Compound I (4.0 mg BID) as indicated by the Day 10 mean FEV₁/FVC data. This corresponds to an improvement in FEV₁/FVC in subjects independent of baseline FeNO levels.

### Placebo-Corrected Changes in FEF₂₅₋₇₅ (Overall COPD Population)

Placebo-corrected changes in FEF₂₅₋₇₅ (post-bronchodilator) are reported below in Table 20 for COPD patients treated with Compound I (4 mg BID).

TABLE 20

| Timepoint | Mean (L/sec) (n = 9) | Max improvement (L/sec) |
|---|---|---|
| Day 10 pre-dose | +0.15 | +0.35 |
| Day 10 + 0.5 hours | +0.22 | +0.32 |
| Day 10 + 1 hours | +0.14 | +0.33 |
| Day 10 + 2 hours | +0.17 | +0.44 |
| Day 10 + 4 hours | +0.17 | +0.40 |
| Day 10 + 8 hours | +0.22 | +0.60 |

The results in Table 20 indicate that improvements in FEF₂₅₋₇₅ were achieved post-administration of Compound I (4.0 mg BID) as indicated by the Day 10 mean FEV₁/FVC data. This corresponds to an improvement in FEV₁/FVC in subjects independent of baseline FeNO levels.

### Lung Function Changes (Day 1 Pre-Dose to Mean Days 10-12; Overall COPD Population)

Lung function changes (post-bronchodilator) are reported below in Table 21 (T2 low subjects) and Table 22 (T2 high subjects) for COPD patients treated with Compound I (4 mg BID). Table 21 shows that all patients with T2 low inflammation (100% or 6/6) achieved improved placebo-corrected mean Day 10-12 FEV₁. Also, the majority of patients with T2 low inflammation (83% or 5/6) achieved improved absolute mean Day 10-12 FEV₁ (Table 21).

TABLE 21

| | Day 1 | | | | |
|---|---|---|---|---|---|
| | pre-dose | | | FEF₂₅₋₇₅ change | |
| | blood | FEV₁ change (mL) | | (L/sec) | |
| Patient | eosinophils | Abso- | Placebo- | Abso- | Placebo- |
| ID | (cells/μL) | lute | corrected | lute | corrected |
| 1 | 70 | 90 | 210 | 0.01 | 0.11 |
| 2 | 220 | 140 | 260 | −0.01 | 0.09 |
| 3 | 80 | 190 | 310 | 0.11 | 0.21 |
| 4 | 200 | 80 | 200 | 0.04 | 0.14 |
| 5 | 170 | 300 | 420 | 0.30 | 0.40 |
| 6 | 160 | −10 | 110 | 0.03 | 0.13 |
| Mean | 150 | 130 | 250 | 0.08 | 0.18 |
| Improvement | Not | 80-300 | 110-420 | 0.01-0.30 | 0.09-0.40 |
| Range | Applicable | | | | |

TABLE 22

| | Day 1 | | | | |
|---|---|---|---|---|---|
| | pre-dose | | | | |
| | blood | FEV₁ change (mL) | | FEF₂₅₋₇₅ change (L/sec) | |
| Patient | eosinophils | Abso- | Placebo- | Abso- | Placebo- |
| ID | (cells/μL) | lute | corrected | lute | corrected |
| 7 | 480 | 170 | 290 | 0.03 | 0.13 |
| 8 | 330 | −100 | 20 | 0.05 | 0.15 |
| Mean | 405 | 40 | 160 | 0.04 | 0.14 |

The data in Table 21 and 22 indicate that subjects with both T2 low and T2 high COPD phenotypes achieve improved placebo-corrected mean Day 10-12 FEV₁ and placebo-corrected Day 10-12 FEF₂₅₋₇₅ on treatment with Compound I (4 mg BID). Further, a comparison of mean values in Table 21 and 22 suggests that subjects with T2 low COPD achieve greater improvements in placebo-corrected Day 10-12 FEV₁ and placebo-corrected Day 10-12 FEF₂₅₋₇₅ than subjects with T2 high COPD.

It is well known that blood eosinophil count correlates with treatment effectiveness in COPD patients that add an ICS to their existing bronchodilator treatment regimen (Global Initiative for Chronic Obstructive Lung Disease, Global strategy for the diagnosis, management and prevention of chronic obstructive pulmonary disease, Report, 2025). Specifically, patients with a higher blood eosinophil count achieve more benefit from an added ICS than patients with a lower blood eosinophil count. The results in Tables 21 and 22 are unexpected in that Compound I formulations produce greater lung function improvements (e.g., FEV₁ and FEF₂₅₋₇₅) in subjects with T2 low COPD (blood eosinophils <300 cells/μL) than in subjects with T2 high COPD (blood eosinophils ≥300 cells/μL).

### Correlation of Lung Function and FeNO Improvement (Day 1 Re-Dose to Mean Days 10-12 Overall COPD Population)

A correlation of lung function improvement and FeNO improvement (post-bronchodilator) is reported below in Table 23 (T2 low subjects) and Table 24 (T2 high subjects) for COPD patients treated with Compound I (4 mg BID).

TABLE 23

| | | Placebo- | | | |
|---|---|---|---|---|---|
| | Day 1 | corrected | | | |
| | pre-dose | lung function | Day 1 | | |
| | blood | improvement | pre-dose | % FeNO change | |
| Patient | eosinophils | (FEV₁ and | FeNO | | Placebo- |
| ID | (cells/μL) | FEF₂₅₋₇₅) | (ppb) | Absolute | corrected |
| 1 | 70 | Yes | 26.7 | −25.4 | −66.6 |
| 2 | 220 | Yes | 37.3 | −0.8 | −42.0 |
| 3 | 80 | Yes | 17.4 | −9.1 | −50.3 |
| 4 | 200 | Yes | 6.1 | +35.5 | −5.7 |
| 5 | 170 | Yes | 30.3 | +12.2 | −29.0 |
| 6 | 160 | Yes | 23.7 | +4.4 | −36.8 |
| Mean | 150 | | 23.6 | +2.8 | −38.4 |
| Improvement | Not | | 6.1 to 37.3 | −0.8 to −25.4 | −5.7 to −66.6 |
| Range | Applicable | | | | |

TABLE 24

| | | Placebo- | | | |
|---|---|---|---|---|---|
| | Day 1 | corrected | | | |
| | pre-dose | lung function | Day 1 | | |
| | blood | improvement | pre-dose | % FeNO change | |
| Patient | eosinophils | (FEV₁ and | FeNO | | Placebo- |
| ID | (cells/μL) | FEF₂₅₋₇₅) | (ppb) | Absolute | corrected |
| 7 | 480 | Yes | 51.7 | −24.1 | −82.4 |
| 8 | 330 | Yes | 70.8 | −33.7 | −74.8 |
| Mean | 405 | | 61.3 | −28.9 | −78.6 |

The data in Table 23 and 24 indicate that subjects with both T2 low and T2 high COPD phenotypes achieve improved placebo-corrected mean Day 10-12 FeNO levels on treatment with Compound I (4 mg BID), with T2 high subjects achieving a greater improvement.

Although the methods of treatment of the disclosure have been described in some detail by way of illustration and example for purposes of clarity of understanding, one of ordinary skill in the art will appreciate that certain changes and modifications may be practiced within the scope of the appended claims. In addition, each reference, including all of the U.S. patents, U.S. patent application publications, U.S. patent applications, foreign patents, foreign patent applications and non-patent publications referred to in this specification are incorporated herein by reference, in their entirety, to the extent not inconsistent with the present description. Where a conflict exists between the instant application and a reference provided herein, the instant application shall dominate.

1. A method of treating Asthma-COPD Overlap Syndrome (ACOS) in a subject in need thereof, wherein the method comprises administering to the subject a therapeutically effective amount of Compound I, or a pharmaceutically acceptable salt thereof, wherein:
Compound I has the following structure:

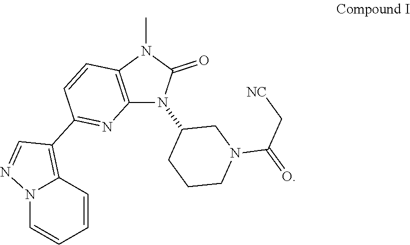

2. A method of treating a disease or disorder in a subject in need thereof, wherein the method comprises administering to the subject a therapeutically effective amount of Compound I, or a pharmaceutically acceptable salt thereof,
wherein:
Compound I has the following structure: 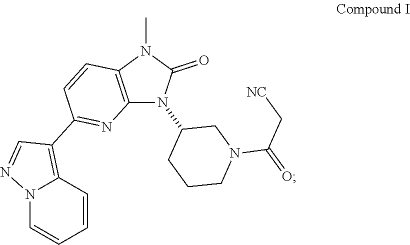
the disease or disorder is selected from asthma, COPD, Asthma-COPD Overlap Syndrome (ACOS), and chronic bronchitis, or a combination thereof; and
prior to administering Compound I, the subject has a blood eosinophil level that is less than about 300 cells/μL.

3. (canceled)

4. The method of claim 2, wherein the disease or disorder is COPD.

5. The method of claim 2, wherein the disease or disorder is chronic bronchitis.

6. (canceled)

7. The method of claim 2, wherein the subject is diagnosed with non-eosinophilic inflammation.

8. (canceled)

9. The method of claim 2, wherein the subject is further diagnosed with non-eosinophilic COPD.

10. (canceled)

11. The method of claim 9, wherein the non-eosinophilic COPD is characterized by a T2 low phenotype.

12-14. (canceled)

15. The method of claim 2, wherein the subject is diagnosed with exercise intolerance.

16. The method of claim 2, wherein the subject is diagnosed with dyspnea.

17. The method of claim 2, wherein, prior to administering Compound I, the subject's dyspnea according to the modified Medical Research Council (mMRC) scale is greater than or equal to 0.

18. The method of claim 2, wherein, prior to administering Compound I, the subject has a COPD assessment test (CAT) score of greater than or equal to 4.

20. The method of claim 2, wherein, prior to administering Compound I, the subject's forced expiratory volume in one second (FEV₁) is less than or equal to about 90% of predicted normal.

21-25. (canceled)

26. The method of claim 2, wherein, prior to administering Compound I, the subject's FEV₁ is less than or equal to about 3.0 L.

27. (canceled)

28. The method of claim 2, wherein prior to administering Compound I, the subject's forced vital capacity (FVC) is less than or equal to about 5.5 L.

29. (canceled)

30. The method of claim 2, wherein prior to administering Compound I, the subject's FVC is less than or equal to about 130% of predicted normal.

31. (canceled)

32. The method of claim 2, wherein, prior to administering Compound I, the subject's ratio of FEV₁ to forced vital capacity (FEV₁/FVC) is less than or equal to about 0.70.

33. (canceled)

34. The method of claim 2, wherein, prior to administering Compound I, the subject's forced expiratory flow between 25% and 75% of forced vital capacity (FEF₂₅₋₇₅) is less than or equal to about 1.5 L/sec.

35-41. (canceled)

42. The method of claim 2, wherein, prior to administering Compound I, the subject's fractional exhaled nitric oxide (FeNO) concentration is greater than or equal to about 5.0 ppb.

43. The method of claim 2, wherein, prior to administering Compound I, the subject's subject's breathlessness, cough, and sputum scale (BCSS) score is greater than or equal to 1.

44. The method of claim 2, wherein, prior to administering Compound I, the subject is undergoing treatment with a background therapy selected from an inhaled corticosteroid, an inhaled long-acting beta-agonist, and an inhaled long-acting muscarinic antagonist, or a combination of two or more thereof.

45. The method of claim 2, wherein, after administering Compound I, the subject's percent change in FeNO concentration is less than or equal to about −0.5%.

46-49. (canceled)

50. The method of claim 2, wherein, after administering Compound I, the subject's percent change in FEV₁ is greater than or equal to about +5%.

51. The method of claim 2, wherein, after administering Compound I, the subject's change in FEV₁ is greater than or equal to about +40 mL.

52. (canceled)

53. The method of claim 2, wherein, after administering Compound I, the subject's percent change in FVC is greater than or equal to about +0.15%.

54. The method of claim 2, wherein, after administering Compound I, the subject's change in FVC is greater than or equal to about +50 L.

55. The method of claim 2, wherein, after administering Compound I, the subject's percent change in FEV₁/FVC is greater than or equal to about +2.0%.

56. The method of claim 2, wherein, after administering Compound I, the subject's change in FEF₂₅₋₇₅ is greater than or equal to about +0.01 L/sec.

57. The method of claim 2, wherein administration of Compound I produces an exposure of Compound I in the subject as indicated by AUC_{0-∞} that is greater than or equal to about 340,000 h·pg/mL.

58. The method of claim 2, wherein administration of Compound I produces an exposure of Compound I in the subject as indicated by AUC_{0-last non-zero} that is greater than or equal to about 340,000 h·pg/mL.

59. The method of claim 2, wherein administration of Compound I produces an exposure of Compound I in the subject as indicated by AUC_{over dosing interval} that is greater than or equal to about 100,000 h·pg/mL.

60. The method of claim 2, wherein administration of Compound I produces an exposure of Compound I in the subject as indicated by AUC_{0-12 hours} that is greater than or equal to about 100,000 h·pg/mL.

61. The method of claim 2, wherein administration of Compound I produces a concentration of Compound I in the subject as indicated by Cₘₐₓ that is greater than or equal to about 10,000 pg/mL.

62. The method of claim 2, wherein administration of Compound I produces a concentration of Compound I in the subject as indicated by C_{trough} that is greater than or equal to about 9,000 pg/mL.

63. The method of claim 2, wherein administration of Compound I produces a half-life (T_{1/2}) of Compound I in the subject that is greater than or equal to about 15 hours.

64-65. (canceled)

66. A compound that is Compound II: 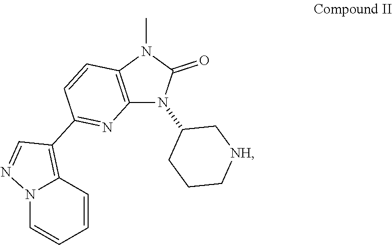
or a pharmaceutically acceptable solvate, hydrate, clathrate, or co-crystal thereof.
